(19) United States
(12) Patent Application Publication
(10) Pub. No.: US 2025/0020748 A1
(43) Pub. Date: Jan. 16, 2025

(54) NON-INVASIVE RADIO-FREQUENCY ANALYTE SENSORS, SYSTEMS AND METHODS

(71) Applicant: KNOW LABS, INC., Seattle, WA (US)

(72) Inventor: JOHN CRONIN, Williston, VT (US)

(21) Appl. No.: 18/765,523

(22) Filed: Jul. 8, 2024

Related U.S. Application Data
(60) Provisional application No. 63/519,454, filed on Aug. 14, 2023.
(60) Provisional application No. 63/519,453, filed on Aug. 14, 2023.
(60) Provisional application No. 63/518,226, filed on Aug. 8, 2023.
(60) Provisional application No. 63/518,218, filed on Aug. 8, 2023.
(60) Provisional application No. 63/516,971, filed on Aug. 1, 2023.
(60) Provisional application No. 63/516,969, filed on Aug. 1, 2023.
(60) Provisional application No. 63/516,968, filed on Aug. 1, 2023.
(60) Provisional application No. 63/516,965, filed on Aug. 1, 2023.
(60) Provisional application No. 63/516,256, filed on Jul. 28, 2023.
(60) Provisional application No. 63/516,254, filed on Jul. 28, 2023.
(60) Provisional application No. 63/516,253, filed on Jul. 28, 2023.
(60) Provisional application No. 63/512,774, filed on Jul. 10, 2023.

(51) Int. Cl. G01R 33/56; A61B 5/055; A61B 5/145; A61B 5/1455; G01R 35/00

(52) U.S. Cl. CPC G01R 33/5608; A61B 5/055; A61B 5/14532; G01R 35/005; A61B 5/1455

(57) ABSTRACT

Non-invasive radio-frequency analyte sensors, systems and methods are described that can be used for measuring one or more analytes using a real-time, non-invasive radio frequency analyte detection device. The sensors, systems and methods provide non-invasive and real-time techniques of measuring analytes and may have applications in various fields, including medical diagnostics and environmental monitoring.

This application claims the benefit of U.S. Provisional Application No. 63/512,774, filed Jul. 10, 2023; U.S. Provisional Application No. 63/516,965, filed Aug. 1, 2023; U.S. Provisional Application No. 63/516,253, filed Jul. 28, 2023; U.S. Provisional Application No. 63/516,254, filed Jul. 28, 2023; U.S. Provisional Application No. 63/516,256, filed Jul. 28, 2023; U.S. Provisional Application No. 63/516,968, filed Aug. 1, 2023; U.S. Provisional Application No. 63/516,969, filed Aug. 1, 2023; U.S. Provisional Application No. 63/516,971, filed Aug. 1, 2023; U.S. Provisional Application No. 63/518,218, filed Aug. 8, 2023; U.S. Provisional Application No. 63/518,226, filed Aug. 8, 2023; U.S. Provisional Application No. 63/519,453, filed Aug. 14, 2023; and U.S. Provisional Application No. 63/519,454, filed Aug. 14, 2023; which applications are incorporated herein by reference in their entirety.

### FIELD

The present disclosure is generally related to systems and methods of monitoring health parameters and, more particularly, relates to a system and a method of monitoring in real-time at least one analyte level, such as glucose levels, using radio frequency signals.

### BACKGROUND

Companies in the healthcare industry do not have the ability to non-invasively detect most blood analytes in real-time. Most blood analysis is invasive, usually requiring the drawing of blood, and slow. While many non-invasive devices measure other health parameters such as heart rate, pulse rate, and sleep cycle, these devices offer limited information on a patient. Data from these non-invasive devices could be put in better context and would likely be more accurate if it could be compared, contrasted, and combined with real-time blood analyte measurements.

A Raman laser is a type of laser that produces light through a process called Raman scattering. In Raman scattering, light interacts with the molecules of a material and transfers some of its energy to the molecules, causing them to vibrate. These vibrations produce new photons, which are emitted as light in a different wavelength than the original light. In a Raman laser, a high-intensity beam of light is directed through a material with a high Raman scattering coefficient. This causes the material to emit light in a new wavelength, which is then amplified through a process called stimulated Raman scattering. The resulting output is a laser beam in a different wavelength than the original light source. Raman lasers have a variety of applications, including in spectroscopy, telecommunications, and medicine. Laser Raman spectroscopy is a highly specific technique that can distinguish between chemical species based on their vibrational spectra. This makes it a valuable tool for identifying and quantifying analytes in complex mixtures, such as biological fluids or environmental samples.

Raman laser technology could provide a non-invasive method to detect blood analytes, but it will likely come with limitations, such as which analytes can be detected, and trade-offs, such as convenience at the cost of accuracy. Combining Raman laser technology with other biometric measurement methods can provide more robust data than one method alone.

Medical professionals and patients often rely on data from single sources or devices, which may not comprehensively understand an individual's health status. Integrating and fusing data from multiple sources would enable the identification of correlations or patterns between analyte levels and other physiological variables, providing more informed diagnostic and treatment decisions.

In the context of noninvasive health monitoring, the accuracy and reliability of measurements can be significantly affected by factors such as patient movement, ambient temperature, electrical interference, and other environmental or physiological parameters. The lack of a system to account for these factors and correct measurement errors in real-time limits the clinical utility and adoption of noninvasive monitoring technologies.

Multimodal spectroscopy combines multiple spectroscopic methods to gain a more complete understanding of a sample's chemical and physical properties. Spectroscopy involves the interaction of light with matter, and the resulting interactions provide information about the sample's properties.

Multimodal spectroscopy typically combines different spectroscopies, such as infrared, Raman, and fluorescence, to provide complementary information about the sample. Each technique provides unique information about the sample, such as its chemical composition, molecular structure, and physical properties. By combining these techniques, researchers can obtain a more comprehensive view of the sample, leading to a better understanding of its properties and potential applications. The development of multimodal spectroscopy has opened up new avenues for research in many areas, including materials science, biology, and medicine. It has enabled researchers to study complex systems that were previously difficult to analyze, such as biological tissues and heterogeneous materials.

Many medical conditions require continuous monitoring of various health parameters, but existing methods may be invasive, uncomfortable, or require frequent hospital visits. Patients and healthcare providers need a non-invasive, comfortable, and convenient way to monitor health parameters, especially for patients who require constant monitoring. Accurate and reliable monitoring of health parameters is critical for making informed medical decisions and providing appropriate treatment. However, existing monitoring methods may be prone to errors, noise, and disruptions due to environmental and patient-related factors.

Healthcare providers and patients face challenges in continuously monitoring vital health parameters non-invasively, resulting in potential gaps in medical data and decreased patient compliance due to discomfort or inconvenience.

Existing non-invasive monitoring solutions may be affected by individual patient factors, such as skin condition, body type, or age, leading to inaccurate or unreliable readings that could compromise the quality of care and overall patient outcomes.

Conventional monitoring systems often lack robust calibration processes that account for real-time data collected from invasive devices and struggle to meet regulatory compliance standards, limiting their ability to deliver precise, reliable health parameter measurements consistently while ensuring patient privacy and data integrity protection.

In addition, traditional health monitoring methods struggle to provide comprehensive, real-time insights into an individual's well-being, leading to delayed or inaccurate health status assessments. Integrating data from multiple health monitoring devices can be challenging, resulting in a fragmented understanding of an individual's health and limiting the effectiveness of targeted interventions. Further, individuals with chronic health conditions often struggle to track and manage their health parameters in a convenient and non-invasive manner. Many existing monitoring devices lack the ability to integrate data from multiple sources, making it difficult to identify correlations or patterns between various physiological variables. Ensuring data security and privacy while transmitting and storing sensitive health information remains a significant challenge in remote health monitoring systems. The calibration of wearable and portable health monitoring devices is often complex, requiring continuous maintenance to ensure accurate readings. Health professionals and individuals could benefit from personalized recommendations on measuring additional health metrics to improve the confidence of their health assessments. Lastly, integrating data from wearable health monitoring devices with advanced AI and ML systems for data analysis is often cumbersome, limiting the potential for more sophisticated health insights.

Currently, an issue with MRI images is that they are highly sensitive to motion, and even small movements can cause blurring or distortion of the image. Patients may move unintentionally due to discomfort or anxiety, or they may be unable to hold still due to medical conditions or disabilities. Also, MRI images are created using strong magnetic fields, which can interact with metal objects in the body and cause artifacts in the resulting images. Patients with metal implants or devices, such as pacemakers or joint replacements, may not be able to undergo MRI or may require special precautions to ensure safety and accurate imaging. Lastly, MRI images have a limited field of view, meaning that only a certain body area can be imaged at one time. This can make capturing a comprehensive view of complex structures or organs difficult and may require multiple scans or specialized imaging techniques. MRI images are highly detailed and complex and require skilled interpretation by trained radiologists or other healthcare professionals. In some cases, image interpretation may be challenging due to factors such as image noise or variations in tissue structure.

In healthcare and wellness monitoring, there is a need for a versatile and portable solution that can be easily integrated into different wearable devices, enabling individuals to conveniently track their health parameters in real-time and across various contexts, such as at home, work, or during physical activities, ultimately promoting better health awareness and decision-making.

In various industrial environments, such as construction and transportation, there is a need to accurately predict and prevent imminent accidents and incidents by closely monitoring workers' or drivers' health parameters and well-being, allowing for timely interventions to ensure their safety.

Traditional methods of monitoring health parameters often require invasive procedures, causing discomfort and inconvenience to individuals, which calls for a non-invasive and real-time solution to accurately measure and assess individuals' health conditions in various settings.

Construction workers, delivery drivers, healthcare workers, farmers, and manufacturing workers face unique challenges affecting their blood glucose levels. For construction workers, physically demanding work requiring movement and exertion can impact blood glucose levels. Similarly, farmers may experience fluctuations in blood sugar due to physically demanding tasks like lifting heavy objects and manual labor. Manufacturing workers also face challenges, often working long hours and performing strenuous tasks such as lifting and carrying heavy objects. On the other hand, delivery drivers may encounter issues with blood sugar levels due to limited physical activity and food access during long driving hours. Healthcare workers, who often work long hours and experience irregular mealtimes, are also at risk of blood glucose level fluctuations. Real-time glucose monitoring can help these professionals regulate their glucose levels, maintain optimal blood sugar levels throughout the day, and avoid hypoglycemia or hyperglycemia, ensuring better health and productivity.

### BRIEF DESCRIPTION OF DRAWINGS

FIG. 1: Illustrates a system for radio frequency health monitoring, according to a first embodiment.

FIG. 2: Illustrates an example operation of a Device Base Module, according to the first embodiment.

FIG. 3: Illustrates an example operation of an Input Waveform Module, according to the first embodiment.

FIG. 4: Illustrates an example operation of a Matching Module, according to the first embodiment.

FIG. 5: Illustrates an example operation of a Machine Learning Module, according to the first embodiment.

FIG. 6: Illustrates an example operation of a Connection Module, according to the first embodiment.

FIG. 7: Illustrates an example operation of a Data Fusion Module, according to the first embodiment.

FIG. 8: Illustrates an example operation of a Report Module, according to the first embodiment.

FIG. 9: Illustrates a system for radio frequency health monitoring, according to a second embodiment.

FIG. 10: Illustrates an example operation of a Device Base Module, according to the second embodiment.

FIG. 11: Illustrates an example operation of an Input Waveform Module, according to the second embodiment.

FIG. 12: Illustrates an example operation of a Matching Module, according to the second embodiment.

FIG. 13: Illustrates an example operation of a Machine Learning Module, according to the second embodiment.

FIG. 14: Illustrates an example operation of a Connection Module, according to the second embodiment.

FIG. 15: Illustrates an example operation of a Data Fusion Module, according to the second embodiment.

FIG. 16: Illustrates an example operation of a Report Module, according to the second embodiment.

FIG. 17: Illustrates an example operation of a Setup Module, according to the second embodiment.

FIG. 18: Illustrates an example operation of an Input Spectrum Module, according to the second embodiment.

FIG. 19: Illustrates an example operation of a Spectrum Matching Module, according to the second embodiment.

FIG. 20: Illustrates an example operation of a Spectrum Machine Learning Module, according to the second embodiment.

FIG. 21: Illustrates a system for radio frequency health monitoring, according to a third embodiment.

FIG. 22: Illustrates an example operation of a Device Base Module, according to the third embodiment.

FIG. 23: Illustrates an example operation of an Input Waveform Module, according to the third embodiment.

FIG. 24: Illustrates an example operation of a Matching Module, according to the third embodiment.

FIG. 25: Illustrates an example operation of a Machine Learning Module, according to the third embodiment.

FIG. 26: Illustrates an example operation of a Connection Module, according to the third embodiment.

FIG. 27: Illustrates an example operation of a Data Fusion Module, according to the third embodiment.

FIG. 28: Illustrates an example operation of a Report Module, according to the third embodiment.

FIG. 29: Schematically illustrates a system for radio frequency health monitoring, according to a fourth embodiment.

FIG. 30: Illustrates an example operation of a Device Base Module, according to the fourth embodiment.

FIG. 31: Illustrates an example operation of an Input Waveform Module, according to the fourth embodiment.

FIG. 32: Illustrates an example operation of a Matching Module, according to the fourth embodiment.

FIG. 33: Illustrates an example operation a Machine Learning Module, according to the fourth embodiment.

FIG. 34: Illustrates an example operation of a Connection Module, according to the fourth embodiment.

FIG. 35: Illustrates an example operation of a Data Fusion Module, according to the fourth embodiment.

FIG. 36: Illustrates an example operation of a Report Module, according to the fourth embodiment.

FIG. 37: Illustrates a system for radio frequency health monitoring, according to a fifth embodiment.

FIG. 38: Illustrates an example operation of a Device Base Module, according to the fifth embodiment.

FIG. 39: Illustrates an example operation of an Input Waveform Module, according to the fifth embodiment.

FIG. 40: Illustrates an example operation of a Matching Module, according to the fifth embodiment.

FIG. 41: Illustrates an example operation of a Machine Learning Module, according to the fifth embodiment.

FIG. 42: Illustrates an example operation of a Connection Module, according to the fifth embodiment.

FIG. 43: Illustrates an example operation of a Data Fusion Module, according to the fifth embodiment.

FIG. 44: Illustrates an example operation of a Report Module, according to the fifth embodiment.

FIG. 45: Illustrates an example of a radio frequency health monitoring system, according to a sixth embodiment.

FIG. 46: Illustrates an example operation of a Device Base Module, according to the sixth embodiment.

FIG. 47: Illustrates an example operation of an Input Waveform Module, according to the sixth embodiment.

FIG. 48: Illustrates an example operation of a Matching Module, according to the sixth embodiment.

FIG. 49: Illustrates an example operation of a Machine Learning Module, according to the sixth embodiment.

FIG. 50: Illustrates an example operation of a Sensor Malfunction Module, according to the sixth embodiment.

FIG. 51: Illustrates an example operation of an Interference Module, according to the sixth embodiment.

FIG. 52: Illustrates an example operation of an Environment Module, according to the sixth embodiment.

FIG. 53: Illustrates an example operation of a Patient Module, according to the sixth embodiment.

FIG. 54: Illustrates an example of a radio frequency health monitoring system, according to a seventh embodiment.

FIG. 55: Illustrates an example operation of a Device Base Module, according to the seventh embodiment.

FIG. 56: Illustrates an example operation of an Input Waveform Module, according to the seventh embodiment.

FIG. 57: Illustrates an example operation of a Matching Module, according to the seventh embodiment.

FIG. 58: Illustrates an example operation of a Machine Learning Module, according to the seventh embodiment.

FIG. 59: Illustrates an example operation of a Connection Module, according to the seventh embodiment.

FIG. 60: Illustrates an example operation of a Calibration Module, according to the seventh embodiment.

FIG. 61: Illustrates an example operation of a Report Module, according to the seventh embodiment.

FIG. 62: Illustrates an example of a radio frequency health monitoring system, according to an eighth embodiment.

FIG. 63: Illustrates an example operation of a Device Base Module, according to the eighth embodiment.

FIG. 64: Illustrates an example operation of an Input Waveform Module, according to the eighth embodiment.

FIG. 65: Illustrates an example operation of a Matching Module, according to the eighth embodiment.

FIG. 66: Illustrates an example operation of a Machine Learning Module, according to the eighth embodiment.

FIG. 67: Illustrates an example operation of a Connection Module, according to the eighth embodiment.

FIG. 68: Illustrates an example operation of a Data Fusion Module, according to the eighth embodiment.

FIG. 69: Illustrates an example operation of an Analysis Module, according to the eighth embodiment.

FIG. 70: Illustrates an example operation of a Calibration Module, according to the eighth embodiment.

FIG. 71: Illustrates an example operation of a Report Module, according to the eighth embodiment.

FIG. 72: Illustrates an example of a radio frequency health monitoring system, according to a ninth embodiment.

FIG. 73: Illustrates an example operation of a Device Base Module, according to the ninth embodiment.

FIG. 74: Illustrates an example operation of an Input Waveform Module, according to the ninth embodiment.

FIG. 75: Illustrates an example operation of a Matching Module, according to the ninth embodiment.

FIG. 76: Illustrates an example operation of a Machine Learning Module, according to the ninth embodiment.

FIG. 77: Illustrates an example operation of a Connection Module, according to the ninth embodiment.

FIG. 78: Illustrates an example operation of a Data Fusion Module, according to the ninth embodiment.

FIG. 79: Illustrates an example operation of an Analysis Module, according to the ninth embodiment.

FIG. 80: Illustrates an example operation of a Calibration Module, according to the ninth embodiment.

FIG. 81: Illustrates an example operation of a Report Module, according to the ninth embodiment.

FIG. 82: Illustrates an example operation of a Simulated Data Module, according to the ninth embodiment.

FIG. 83: Illustrates an example operation of a Recommendation Module, according to the ninth embodiment.

FIG. 84: Illustrates a radio frequency health monitoring system, according to a tenth embodiment.

FIG. 85: Illustrates an example operation of a Device Base Module, according to the tenth embodiment.

FIG. 86: Illustrates an example operation of an Input Waveform Module, according to the tenth embodiment.

FIG. 87: Illustrates an example operation of a Matching Module, according to the tenth embodiment.

FIG. 88: Illustrates an example operation of a Machine Learning Module, according to the tenth embodiment.

FIG. 89: Illustrates an example operation of a Connection Module, according to the tenth embodiment.

FIG. 90: Illustrates an example operation of a Data Fusion Module, according to the tenth embodiment.

FIG. 91: Illustrates an example operation of a Report Module, according to the tenth embodiment.

FIG. 92: Illustrates an example of a Fusion Rules Database, according to the tenth embodiment.

FIG. 93: Illustrates an example of a system for fusing data from a non-invasive RF device with MRI data, according to an eleventh embodiment.

FIG. 94: Illustrates an example operation of a Base Module, according to the eleventh embodiment.

FIG. 95: Illustrates an example operation of a Scan Module, according to the eleventh embodiment.

FIG. 96: Illustrates an example operation of a Location Module, according to the eleventh embodiment.

FIG. 97: Illustrates an example operation of a Fusion Module, according to the eleventh embodiment.

FIG. 98: Illustrates an example operation of an Information Module, according to the eleventh embodiment.

FIG. 99: Illustrates an example of a Threshold Database, according to the eleventh embodiment.

FIG. 100: Illustrates an example of a Patient Database, according to the eleventh embodiment.

FIG. 101: Illustrates an example operation of an MRI Module, according to the eleventh embodiment.

FIG. 102: Illustrates an example of an MRI Database, according to the eleventh embodiment.

FIG. 103: Illustrates a system for radio frequency health monitoring, according to a twelfth embodiment.

FIG. 104: Illustrates an example operation of a Device Base Module, according to the twelfth embodiment.

FIG. 105: Illustrates an example operation of an Input Waveform Module, according to the twelfth embodiment.

FIG. 106: Illustrates an example operation of a Matching Module, according to the twelfth embodiment.

FIG. 107: Illustrates an example operation of a Machine Learning Module, according to the twelfth embodiment.

FIG. 108: Illustrates an example operation of a Connection Module, according to the twelfth embodiment.

FIG. 109: Illustrates an example operation of an Incident Log Module, according to the twelfth embodiment.

FIG. 110: Illustrates an example operation of a Base Module, according to the twelfth embodiment.

FIG. 111: Illustrates an example operation of a Construction Module, according to the twelfth embodiment.

FIG. 112: Illustrates an example operation of a Driver Module, according to the twelfth embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the term module is at least directed to software, hardware, circuitry, and programming. The word “module” shall mean any software program, hardware program, or software and hardware program that performs the stated functions.

U.S. Pat. Nos. 10,548,503, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, are each individually incorporated herein by reference in its entirety.

### Embodiment 1

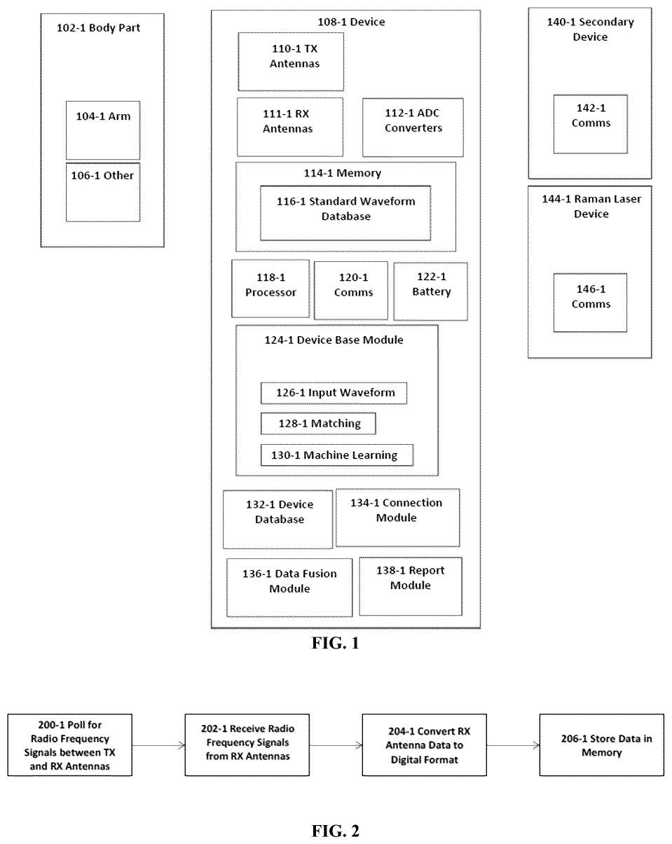

FIG. 1 is a schematic illustration of a system for radio frequency health monitoring. This system is configured to be attached or in proximity to a body part 102-1. The body part 102-1 may be an arm 104-1. The body part 102-1 may be another body part 106-1 besides an arm, such as a leg, finger, chest, head, or any other body part from which useful medical parameters can be taken. The system may comprise a device 108-1, which may be a wearable and portable device such as, but not limited to, a cell phone, a smartwatch, a tracker, a wearable monitor, a wristband, and a personal blood monitoring device. The system may further comprise one or more transmit (“TX”) antennas 110-1. The one or more TX antennas may be configured to transmit RF in the RF Activated Range from 500 MHZ to 300 GHZ. A pre-defined frequency may correspond to a range suitable for the human body in one embodiment. For example, the one or more TX antennas would transmit radio frequency signals at a range of 120-126 GHz.

The system may further comprise one or more receive (“RX”) antennas 111-1. The one or more RX antennas may be configured to receive the RF signals in response to the RF signal transmitted from the one or more TX antennas.

The system may further comprise an ADC converter 112-1, which may be configured to convert the received RF signals from an analog signal into a digital processor readable format.

The system may further comprise memory 114-1, which may be configured to store the transmitted RF signals by the one or more TX antennas 110-1 and store the received portion of the response or responded RF signals from the one or more RX antennas 111-1. Further, the memory 114-1 may also store the converted digital processor readable format by the ADC converter 112-1. The memory 114-1 may include suitable logic, circuitry, and/or interfaces that may be configured to store a machine code and/or a computer program with at least one code section executable by the processor 118-1. Examples of implementation of the memory 114-1 may include, but are not limited to, Random Access Memory (RAM), Read Only Memory (ROM), Hard Disk Drive (HDD), and/or a Secure Digital (SD) card.

The system may further comprise a standard waveform database 116-1, which may contain standard waveforms for known patterns. These may be raw or converted device readings from patients or persons with known conditions. For example, the standard waveform database 116-1 may include raw or converted device readings from the right arm of a patient known to have diabetes or an average of multiple patients. This data can be compared to readings from a person with an unknown condition in order to determine if the waveforms taken from that person match any of the known standard waveforms.

The system may further comprise a processor 118-1, which may facilitate the operation of the device 108-1 according to the instructions stored in the memory 114-1. The processor 118-1 may include suitable logic, circuitry, interfaces, and/or code that may be configured to execute a set of instructions stored in the memory 114-1.

The system may further comprise a comms 120-1, which may be a communication element such as a Wi-Fi or RFC transmitter and receiver or physical connection that can send and receive data to other devices of the system and devices external to the system. The comms 120-1 may be configured to comply with regulatory acts such as HIPPA. For example, the comms 120-1 may include features such as encryption, access control, audit trails, secure transmission, credential verification, or other features which may protect the integrity and privacy of transmitted or received data.

The system may further comprise a battery 122-1, which may power hardware modules of the device 108-1. The device 108-1 may be configured with a charging port to recharge the battery 122-1. Charging of the battery 122-1 may be wired or wireless.

The system may further comprise a device base module 124-1, which may be configured to store instructions for executing the computer program from the converted digital processor readable format of the ADC converter 112-1. The device base module 124-1 may be configured to facilitate the operation of the processor 118-1, the memory 114-1, the one or more TX antennas 110-1, the one or more RX antennas 111-1, and the comms 120-1. Further, the device base module 124-1 may be configured to create polling of the RF Activated Range from 500 MHZ to 300 GHZ. It can be noted that the device base module 124-1 may be configured to filter the RF Activated Range from 500 MHZ to 300 GHZ received from one or more RX antennas 111-1.

The system may further comprise an input waveform module 126-1, which may extract a radio frequency waveform from memory. This may be the raw or converted recording of data from the one or more RX antennas 111-1 from a patient wearing the device. If the entire radio frequency is too long for effective matching, the input waveform module 126-1 may select a time interval within the data set. This input waveform may then be sent to the matching module 128-1.

The system may further comprise a matching module 128-1, which may match the input waveform and each of the standard waveforms in the standard waveform database 116-1 by performing a convolution and/or cross-correlation of the input waveform and the standard waveform. These convolutions and/or cross-correlations are then sent to the machine learning module.

The system may further comprise a machine learning module 130-1 which has been trained to identify health parameters based on the convolution and/or cross-correlations of the input and standard waveforms. The machine learning module 130-1 receives the convolutions and cross-correlations from the matching module 128-1 and outputs any health parameters identified.

The system may further comprise a device database 132-1, which may store the health parameters output by the machine learning module 130-1 and data collected from other devices such as the secondary device 140-1 and Raman laser device 144-1.

The system may further comprise a connection module 134-1, which may connect to the secondary device 140-1 and Raman laser device 144-1 and collect data which then may be stored in the device database 132-1.

The system may further comprise a data fusion module 136-1, which may fuse the real-time analyte data from the device 108-1 and collected data from the secondary device 140-1 and Raman laser device 144-1 stored in the device database 132-1. Fusing data may refer to the process of combining multiple sources of information or data sets into a single integrated output. This process involves aligning and combining data from different sources while ensuring that it remains accurate and consistent. For example, fusing data from the non-invasive RF analyte detection device and other physiological devices, such as heart rate, sleep system, or pulse rate devices, would enable the identification of correlations or patterns that may exist between the analyte levels and other physiological variables. Such information can be useful in providing a more comprehensive understanding of the individual's health status and may aid in the development of targeted interventions or treatments.

The system may further comprise a report module 138-1, which may display a report of the fused data from the data fusion module 136-1. This report may be sent to an email address, displayed on a screen, sent to another module, or otherwise reported.

The system may further comprise a secondary device 140-1, which may be a heart rate device, a sleep system device, a pulse rate device, or a sensor inserted under the skin to measure glucose level or any other device that produces data which may be collected by the connection module 134-1.

The system may further comprise the comms 142-1, which may be a communication element such as a Wi-Fi or RFC transmitter and receiver or physical connection that can send and receive data to other devices of the system and devices external to the system. The comms 142-1 may be configured to comply with regulatory acts such as HIPPA. For example, the comms 142-1 may include features such as encryption, access control, audit trails, secure transmission, credential verification, or other features which may protect the integrity and privacy of transmitted or received data.

The system may further comprise a Raman laser device 144-1, which may be a wearable device which uses a Raman laser or lasers for non-invasive spectroscopy. The Raman laser device 144-1 may be worn on the patient's wrist or any other suitable location. The Raman laser device 144-1 may comprise a Raman laser and a detector that may be configured to detect the Raman signal from the patient's blood. The device 144-1 may also include a processor and a memory that may be configured to analyze the Raman signal and determine the concentration of the analytes in the patient's blood. In operation, the Raman laser may be directed onto the patient's skin, and the detector may detect the Raman signal generated by the laser's interaction with the patient's blood. The processor may analyze the signal to determine the concentration of various analytes in the patient's blood. The Raman laser device 144-1 may be calibrated to provide accurate measurements of the concentration of the analytes in the patient's blood. The calibration may be performed using blood samples obtained from the patient, which may be analyzed using conventional methods. The Raman laser device 144-1 may be used to monitor the concentration of analytes in the patient's blood continuously or at regular intervals. The Raman laser device 144-1 may provide real-time feedback to the patient or healthcare provider, which may enable them to take appropriate action based on the readings obtained from the device. The Raman laser device 144-1 may have a compact and portable design, allowing for easy use and transport. The device may also have a user-friendly interface, displaying the concentration of analytes in an easy-to-understand format for both patients and healthcare providers. The Raman laser device 144-1 may use a single or multiple laser to generate Raman signals from the patient's blood. The lasers may have different wavelengths to target different types of analytes, and the device may also include filters to reduce interference from background signals. To ensure patient safety, the Raman laser device 144-1 may use low-power laser sources and may comply with relevant safety standards and regulations. The Raman laser device 144-1 may also be designed to minimize discomfort or irritation to the patient's skin during use. The Raman laser device 144-1 may be used in various medical applications, including diabetes management, monitoring metabolic disorders, and detecting drug levels in the blood.

The system may further comprise the comms 146-1, which may be a communication element such as a Wi-Fi or RFC transmitter and receiver or physical connection that can send and receive data to other devices of the system and devices external to the system. The comms 146-1 may be configured to comply with regulatory acts such as HIPPA. For example, the comms 146-1 may include features such as encryption, access control, audit trails, secure transmission, credential verification, or other features which may protect the integrity and privacy of transmitted or received data.

FIG. 2 illustrates an example operation of the device base module 124-1. The process may begin with the device base module 124-1 polling the Active Range RF signals between the one or more TX antennas 110-1 and the one or more RX antennas 111-1 at step 200-1. The device base module 124-1 may be configured to read and process instructions stored in the memory 114-1 using the processor 118-1. The one or more TX antennas 110-1 may be configured to transmit RF in the RF Activated Range from 500 MHZ to 300 GHZ. For example, the one or more TX antennas 110-1 may use RF signals at a range of 500 MHZ to 300 GHZ. The device base module 124-1 may receive the RF frequency signals from the one or more RX antennas 111-1 at step 202-1. For example, one or more RX antennas 111-1 receives a response or responded RF of frequency range 300-330 GHz from the patient's blood. The device base module 124-1 may be configured to convert the received RF signals into a digital format using the ADC 112-1 at step 204-1. For example, the received 300-330 GHz frequency range RF is converted into a 10-bit data signal. The device base module 124-1 may be configured to store converted digital format into the memory 114-1 at step 206-1.

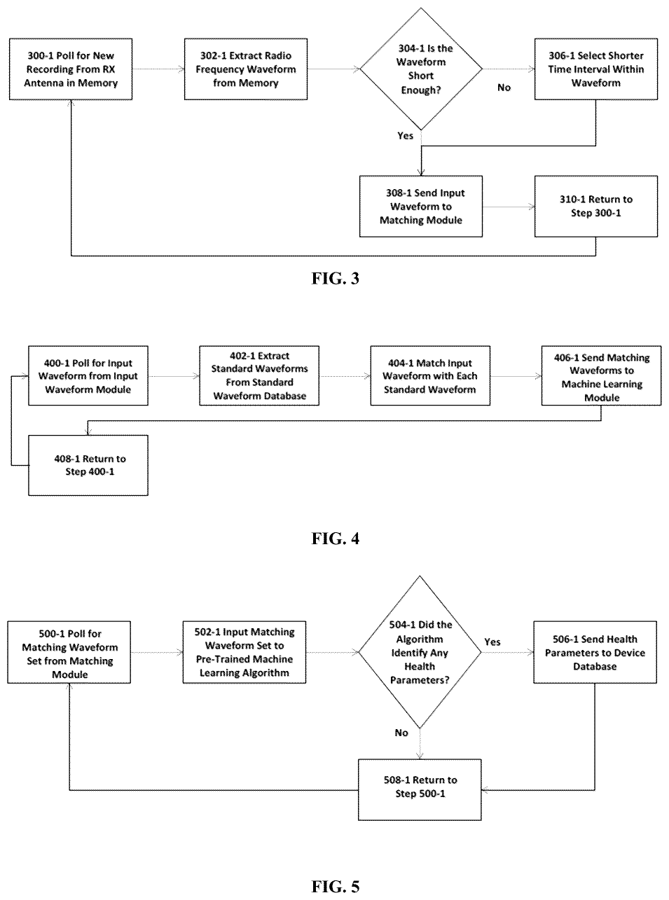

FIG. 3 illustrates an example operation of the input waveform module 126-1. The process may begin with the input waveform module 126-1 polling, at step 300-1, for newly recorded data from the one or more RX antennas 111-1 stored in memory 114-1. The input waveform module 126-1 may extract, at step 302-1, the recorded radio frequency waveform from memory. If there is more than one waveform recorded, the input waveform module 126-1 may select each waveform separately and loop through the following steps. The input waveform module 126-1 may determine, at step 304-1, if the waveform is small enough to be an input waveform for the matching module 128-1. This will depend on the computational requirements and/or restrictions of the matching module 128-1. If the waveform is short enough, the input waveform module 126-1 may skip to step 308-1. If the waveform is too long, the input waveform module 126-1 may select, at step 306-1, a shorter time interval within the entire recorded waveform. For example, if the waveform is 5 minutes long, then only a 30-second interval may be selected. The interval may be selected at random or by a selection process. The input waveform module 126-1 may send, at step 308-1, the input waveform to the matching module 128-1. The input waveform module 126-1 may return, at step 310-1, to step 300-1.

FIG. 4 illustrates an example operation of the matching module 128-1. The process may begin with the matching module 128-1 polling, at step 400-1, for an input waveform from the input waveform module 126-1. The matching module 128-1 may extract, at step 402-1, each standard waveform from the standard waveform database 116-1. The matching module 128-1 may match, at step 404-1, the input waveform with each standard waveform. Matching may be determining which standard waveforms the input waveform is similar to. Matching may involve convolution and/or cross-correlation of the waveforms or any other suitable matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a value of similarity between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function whose values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-1. The matching module 128-1 may send, at step 406-1, the matching waveforms to the machine learning module 130-1. Matching waveforms may refer to the standard waveforms similar to the input waveform, the waveforms generated via convolution and/or cross-correlation, or both. The matching module 128-1 may return, at step 408-1, to step 400-1.

FIG. 5 illustrates an example operation of the machine learning module 130-1. The process may begin with the machine learning module 130-1 polling, at step 500-1, for a set of matching waveforms from the matching module 128-1. Matching waveforms may be a set of standard waveforms similar to the input waveform or statistical combinations of the input waveform and standard waveforms, such as convolutions or cross-correlations. The machine learning module 130-1 may input, at step 502-1, the set of received waveforms into a pre-trained machine learning algorithm. The machine learning algorithm may be trained on similar sets of matched waveforms where the input waveform is from a patient whose health parameters are known. The waveforms may be input directly into the algorithm, such as a set of X and Y values. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During the training process, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-1 may determine, at step 504-1, if the algorithm identified any health parameters. Identification may require a certain interval of confidence. For example, if the machine learning algorithm determines that it is more than 70% likely that a health parameter is correct, that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dl may be identified. If none of the results from the machine learning algorithm are above the confidence threshold, or the results are otherwise inconclusive, the machine learning module 130-1 may skip to step 508-1. If any health parameters were identified, the machine learning module 130-1 may send, at step 506-1, the health parameters to the device database 132-1. The machine learning module 130-1 may return, at step 508-1, to step 500-1.

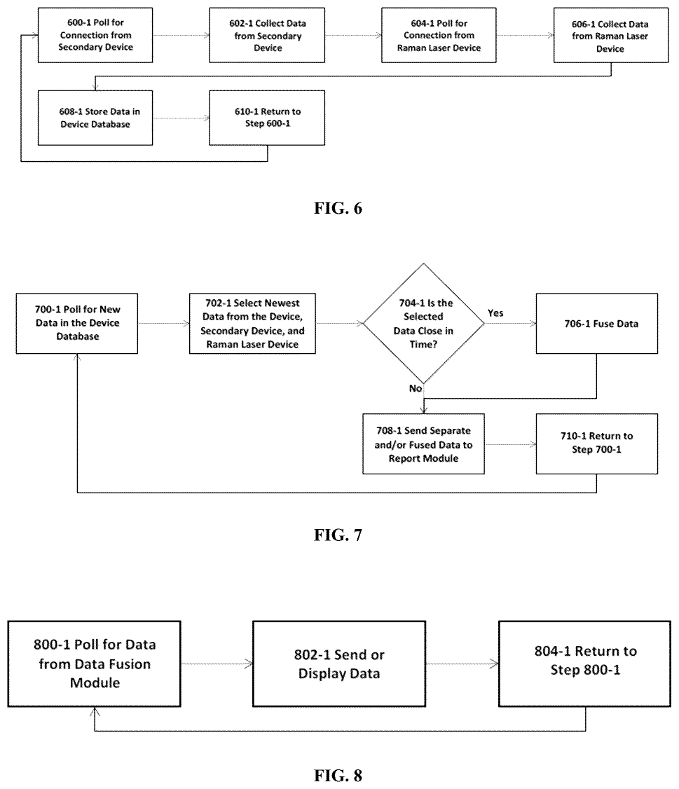

FIG. 6 illustrates an example operation of the connection module 134-1. The process may begin with the connection module 134-1 polling, at step 600-1, for a connection from the secondary device 140-1. If no connection is detected, the connection module 134-1 may search for a secondary device that is attempting to connect or may skip to step 604-1. The connection module 134-1 may collect, at step 602-1, data from the secondary device. This may be real-time data or periodically updated data. Examples of secondary devices may include a heart rate device, sleep system device, pulse rate device, sensor inserted under the skin to measure glucose level or any other device that produces relevant data. The connection module 134-1 may poll, at step 604-1, for a connection from the Raman laser device 144-1. If no connection is detected the connection module 134-1 may search for a Raman laser device 144-1 that is attempting to connect or may skip to step 608-1. The connection module 134-1 may begin at this step if a secondary device 140-1 is unavailable or unnecessary. The connection module 134-1 may collect, at step 606-1, data from the Raman laser device 144-1. This may be real-time data or periodically updated data. The connection module 134-1 may store, at step 608-1, the data from the secondary device 140-1 and/or Raman laser device 144-1 in the device database 132-1. The connection module 134-1 may add contextual data such as the ID of the secondary device 140-1, ID of the Raman laser device 144-1, type of secondary device 140-1, analyte measured by the Raman laser device 144-1, time data was retrieved, etc., to the received data before storage in the device database 132-1. The connection module 134-1 may return, at step 610-1, to step 600-1.

FIG. 7 illustrates an example operation of the data fusion module 136-1. The process may begin with the data fusion module 136-1 polling, at step 700-1, for new data in the device database. This new data may be data from the device 108-1, the secondary device 140-1, or the Raman laser device 144-1. The data fusion module 136-1 may select, at step 702-1, the newest data from the device 108-1, the newest data from the secondary device 140-1, and the newest data from the Raman laser device 144-1. This may be a single data point or a set of data. The data fusion module 136-1 may determine, at step 704-1, if the selected data is close in time. Close in time may mean each piece or set of data is timestamped within a few seconds of each other. For example, the newest data from the device 108-1 may be real-time, but the newest reading from the Raman laser device 144-1 may have been 10 minutes ago. This data would not be close in time if the threshold is a few seconds. The threshold may be varied based on the health parameter being measured. For example, if the secondary device 140-1 is recording sleep cycle activity, then recordings from 10 minutes ago may be relevant because of the gradual nature of sleep cycles, but if the Raman laser device 144-1 is recording blood glucose or electrolyte levels, then data from minutes ago may be highly inaccurate. The time threshold for what data is considered close in time may be set by the user of the device, manufacturer of the device, and/or another module. If the selected data is not close in time, the data fusion module may skip to step 708-1. The data fusion module 136-1 may fuse, at step 706-1, the selected data from the device 108-1, secondary device 140-1, and/or Raman laser device 144-1. Data fusion may describe multiple processes, and which process is performed may depend on the data types being compared. For example, if both pieces or data sets are measurements of the same parameter, such as glucose, then fusing the data may refer to aligning and combining data to ensure it is accurate and consistent. Aligning may be taking the mean or the median. The data may be enhanced, meaning an error is returned if both measurements are outside a predetermined normal range. For another example, fused data may be a correlation function of two sets of data, such as correlating real-time glucose measurements from the device 108-1 to sleep cycle readings from the secondary device 140-1 in order to track the effect of blood glucose on sleep quality. Fusing the data may refer to adjusting one piece or set of data based on the other. For example, pulse rate readings from the secondary device 140-1 may be used to adjust SPO2 readings from the Raman laser device 144-1. which are calibrated for a normal pulse rate. Fusing the data may refer to generating additional health parameters from the combination of the data. For example, blood glucose measurements from the device 108-1, blood cortisol levels from the Raman laser device 144-1, and heart rate measurements from the secondary device 140-1 can be fused to generate stress or activity level. Fusing data may refer to estimating a risk level based on the data sets. For example, blood glucose data from the device 108-1, heartrate data from the secondary device 140-1, and tissue oxygenation data from the Raman laser device 144-1 may all be factored into a risk score. The data fusion module 136-1 may use statistical algorithms, machine learning, and/or AI to perform the data fusion. The data fusion module 136-1 may send, at step 708-1, each piece or set of data and/or the fused data, if available, to the report module 138-1. The data fusion module 136-1 may return, at step 710-1, to step 700-1.

FIG. 8 illustrates an example operation of the report module 138-1. The process may begin with the report module 138-1 polling, at step 800-1, for data from the data fusion module 136-1. The report module 138-1 may send and/or display, at step 802-1, the received data. If a display is connected to the device 108-1 or can be reached by other means, the report module 138-1 may display the data directly. If not, the report module may send data in real-time or periodically. Data may be sent to an email address, a database, a module, or any other destination. The data may be sent to and/or displayed at multiple locations. The report module 138-1 may return, at step 804-1, to step 800-1.

In one example implementation of Embodiment 1, a method for measuring one or more analytes using a real-time, non-invasive radio frequency (“RF”) analyte detection device can include the following: providing a real-time, non-invasive RF analyte detection device; providing a wearable integrated Raman Laser analyte detection device; providing a connection module which connects the real-time, non-invasive RF analyte detection device, the wearable integrated Raman Laser analyte detection device and, at least one other device; storing a first set of data of the one or more analytes obtained from a measurement by the real-time, non-invasive RF analyte detection device; storing a second set of data of the one or more analytes obtained from a measurement by the wearable integrated Raman Laser analyte detection device; storing a third set of data obtained from the at least one other device; executing a fusion module to form fused data from at least two sets of data selected from the group consisting of the first set of data, the second set of data, and the third set of data; and reporting the fused data results.

### Embodiment 2

FIG. 9 is a schematic illustration of a system for radio frequency health monitoring. This system is configured to be attached or in proximity to a body part 102-2. The body part 102-2 may be an arm 104-2. The body part 102-2 may be another body part 106-2 besides an arm, such as a leg, finger, chest, head, or any other body part from which useful medical parameters can be taken. The system may comprise a device 108-2, which may be a wearable and portable device such as, but not limited to, a cell phone, a smartwatch, a tracker, a wearable monitor, a wristband, and a personal blood monitoring device.

The system may further comprise one or more transmit (“TX”) antennas 110-2. The one or more TX antennas may be configured to transmit RF in the RF Activated Range from 500 MHZ to 300 GHZ. A pre-defined frequency may correspond to a range suitable for the human body in one embodiment. For example, the one or more TX antennas would transmit radio frequency signals at a range of 120-126 GHz.

The system may further comprise one or more receive (“RX”) antennas 111-2. The one or more RX antennas may be configured to receive the RF signals in response to the RF signal transmitted from the one or more TX antennas.

The system may further comprise an ADC converter 112-2, which may be configured to convert the received RF signals from an analog signal into a digital processor readable format.

The system may further comprise memory 114-2, which may be configured to store the transmitted RF signals by the one or more TX antennas 110-2 and store the received portion of the response or responded RF signals from the one or more RX antennas 111-2. Further, the memory 114-2 may also store the converted digital processor readable format by the ADC converter 112-2. The memory 114-2 may include suitable logic, circuitry, and/or interfaces that may be configured to store a machine code and/or a computer program with at least one code section executable by the processor 118-2. Examples of implementation of the memory 114-2 may include, but are not limited to, Random Access Memory (RAM), Read Only Memory (ROM), Hard Disk Drive (HDD), and/or a Secure Digital (SD) card.

The system may further comprise a standard waveform database 116-2, which may contain standard waveforms for known patterns. These may be raw or converted device readings from patients or persons with known conditions. For example, the standard waveform database 116-2 may include raw or converted device readings from the right arm of a patient known to have diabetes or an average of multiple patients. This data can be compared to readings from a person with an unknown condition in order to determine if the waveforms taken from that person match any of the known standard waveforms.

The system may further comprise a processor 118-2, which may facilitate the operation of the device 108-2 according to the instructions stored in the memory 114-2. The processor 118-2 may include suitable logic, circuitry, interfaces, and/or code that may be configured to execute a set of instructions stored in the memory 114-2.

The system may further comprise a comms 120-2, which may be a communication element such as a Wi-Fi or RFC transmitter and receiver or physical connection that can send and receive data to other devices of the system and devices external to the system. The comms 120-2 may be configured to comply with regulatory acts such as HIPPA. For example, the comms 120-2 may include features such as encryption, access control, audit trails, secure transmission, credential verification, or other features which may protect the integrity and privacy of transmitted or received data.

The system may further comprise a battery 122-2, which may power hardware modules of the device 108-2. The device 108-2 may be configured with a charging port to recharge the battery 122-2. Charging of the battery 122-2 may be wired or wireless.

The system may further comprise a device base module 124-2, which may be configured to store instructions for executing the computer program from the converted digital processor readable format of the ADC converter 112-2. The device base module 124-2 may be configured to facilitate the operation of the processor 118-2, the memory 114-2, the one or more TX antennas 110-2, the one or more RX antennas 111-2, and the comms 120-2. Further, the device base module 124-2 may be configured to create polling of the RF Activated Range from 500 MHZ to 300 GHZ. It can be noted that the device base module 124-2 may be configured to filter the RF Activated Range from 500 MHZ to 300 GHZ received from one or more RX antennas 111-2.

The system may further comprise an input waveform module 126-2, which may extract a radio frequency waveform from memory. This may be the raw or converted recording of data from the one or more RX antennas 111-2 from a patient wearing the device. If the entire radio frequency is too long for effective matching, the input waveform module 126-2 may select a time interval within the data set. This input waveform may then be sent to the matching module 128-2. The system may further comprise a matching module 128-2, which may match the input waveform and each of the standard waveforms in the standard waveform database 116-2 by performing a convolution and/or cross-correlation of the input waveform and the standard waveform. These convolutions and/or cross-correlations are then sent to the machine learning module.

The system may further comprise a machine learning module 130-2 which has been trained to identify health parameters based on the convolution and/or cross-correlations of the input and standard waveforms. The machine learning module 130-2 receives the convolutions and cross-correlations from the matching module 128-2 and outputs any health parameters identified.

The system may further comprise a device database 132-2, which may store the health parameters output by the machine learning module 130-2 and data collected from other devices such as the secondary device 140-2 and Raman laser device 144-2.

The system may further comprise a connection module 134-2, which may connect to the Raman laser device 144-2, send data from the machine learning module 130-2, and collect data from the spectrum machine learning module 156-2 which then may be stored in the device database 132-2.

The system may further comprise a data fusion module 136-2, which may fuse the real-time analyte data from the device 108-2 and collected data from the secondary device 140-2 and Raman laser device 144-2 stored in the device database 132-2. Fusing data may refer to the process of combining multiple sources of information or data sets into a single integrated output. This process involves aligning and combining data from different sources while ensuring it remains accurate and consistent. For example, fusing data from the non-invasive RF analyte detection device and other physiological devices, such as heart rate, sleep system, or pulse rate devices, would enable the identification of correlations or patterns that may exist between the analyte levels and other physiological variables. Such information can be useful in providing a more comprehensive understanding of the individual's health status and may aid in the development of targeted interventions or treatments.

The system may further comprise a report module 138-2, which may display a report of the fused data from the data fusion module 136-2. This report may be sent to an email address, displayed on a screen, sent to another module, or otherwise reported.

The system may further comprise a secondary device 140-2, which may be a heart rate device, a sleep system device, a pulse rate device, or a sensor inserted under the skin to measure glucose level, or any other device that produces data which may be collected by the connection module 134-2.

The system may further comprise the comms 142-2, which may be a communication element such as a Wi-Fi or RFC transmitter and receiver or physical connection that can send and receive data to other devices of the system and devices external to the system. The comms 142-2 may be configured to comply with regulatory acts such as HIPPA. For example, the comms 142-2 may include features such as encryption, access control, audit trails, secure transmission, credential verification, or other features which may protect the integrity and privacy of transmitted or received data.

The system may further comprise a Raman laser device 144-2, which may be a wearable device which uses a Raman laser or lasers for non-invasive spectroscopy. The Raman laser device 144-2 may be worn on the patient's wrist or any other suitable location. The Raman laser device 144-2 may comprise a Raman laser and a detector that may be configured to detect the Raman signal from the patient's blood. The device 144-2 may also include a processor and a memory that may be configured to analyze the Raman signal and determine the concentration of the analytes in the patient's blood. In operation, the Raman laser may be directed onto the patient's skin, and the detector may detect the Raman signal generated by the laser's interaction with the patient's blood. The processor may analyze the signal to determine the concentration of various analytes in the patient's blood. The Raman laser device 144-2 may be calibrated to provide accurate measurements of the concentration of the analytes in the patient's blood. The calibration may be performed using blood samples obtained from the patient, which may be analyzed using conventional methods. The Raman laser device 144-2 may be used to monitor the concentration of analytes in the patient's blood continuously or at regular intervals. The Raman laser device 144-2 may provide real-time feedback to the patient or healthcare provider, which may enable them to take appropriate action based on the readings obtained from the device. The Raman laser device 144-2 may have a compact and portable design, allowing for easy use and transport. The device may also have a user-friendly interface, displaying the concentration of analytes in an easy-to-understand format for both patients and healthcare providers. The Raman laser device 144-2 may use a single laser or multiple lasers to generate Raman signals from the patient's blood. The lasers may have different wavelengths to target different types of analytes, and the device may also include filters to reduce interference from background signals. To ensure patient safety, the Raman laser device 144-2 may use low-power laser sources and may comply with relevant safety standards and regulations. The Raman laser device 144-2 may also be designed to minimize discomfort or irritation to the patient's skin during use. The Raman laser device 144-2 may be used in various medical applications, including diabetes management, monitoring of metabolic disorders, and detection of drug levels in the blood.

The system may further comprise Raman Laser Device (RLD) comms 146-2, which may be a communication element such as a Wi-Fi or RFC transmitter and receiver or physical connection that can send and receive data to other devices of the system and devices external to the system. The RLD comms 146-2 may be configured to comply with regulatory acts such as HIPPA. For example, the RLD comms 146-2 may include features such as encryption, access control, audit trails, secure transmission, credential verification, or other features which may protect the integrity and privacy of transmitted or received data.

The system may further comprise a setup module 148-2, which may set the initial state of the Raman laser device 144-2 based on data from the device 108-2. The initial state may include which analyte is being detected and which wavelengths of electromagnetic radiation (EMR) should be transmitted in order to detect that analyte.

The system may further comprise an input spectrum module 150-2, which receives the input spectrum which is the spectrum of EMR detected by the Raman laser device 144-2. The input spectrum may then undergo data processing to eliminate noise, artifacts, or other sources of error.

The system may further comprise a spectrum matching module 152-2, which may match the input spectrum and each of the standard spectra in the standard spectrum database 156-2 by performing a convolution and/or cross-correlation of the input spectrum and the standard spectrum. These convolutions and/or cross-correlations are then sent to the spectrum machine learning module.

The system may further comprise a spectrum machine learning module 154-2 which has been trained to identify health parameters based on the convolution and/or cross-correlations of the input and standard spectra. The spectrum machine learning module 154-2 receives the convolutions and cross-correlations from the spectrum matching module 152-2 and sends any health parameters identified to the connection module 134-2 of the device 108-2 via the RLD comms 146-2.

The system may further comprise a standard spectrum database 156-2, which may contain standard spectra for known patterns. These may be raw or converted device readings from patients or persons with known conditions. For example, the standard spectrum database 156-2 may include raw or converted device readings from the right arm of a patient known to have diabetes or an average of multiple patients. This data can be compared to readings from a person with an unknown condition to determine if the spectrum taken from that person matches any of the known standard spectra.

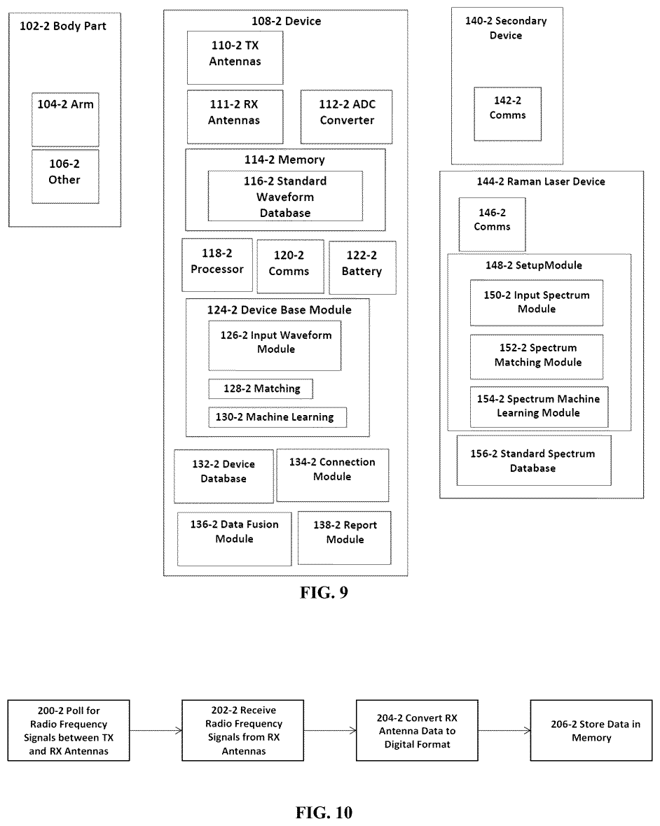

FIG. 10 illustrates an example operation of the device base module 124-2. The process may begin with the device base module 124-2 polling the Active Range RF signals between the one or more TX antennas 110-2 and the one or more RX antennas 111-2 at step 200-2. The device base module 124-2 may be configured to read and process instructions stored in the memory 114-2 using the processor 118-2. The one or more TX antennas 110-2 may be configured to transmit RF in the RF Activated Range from 500 MHZ to 300 GHZ. For example, the one or more TX antennas 110-2 may use RF signals at a range of 500 MHZ to 300 GHZ. The device base module 124-2 may receive the RF frequency signals from the one or more RX antennas 111-2 at step 202-2. For example, one or more RX antennas 111-2 receives a response or responded RF of frequency range 300-330 GHz from the patient's blood. The device base module 124-2 may be configured to convert the received RF signals into a digital format using the ADC 112-2 at step 204-2. For example, the received RF of 300-330 GHz frequency range is converted into a 10-bit data signal. The device base module 124-2 may be configured to store converted digital format into the memory 114-2 at step 206-2.

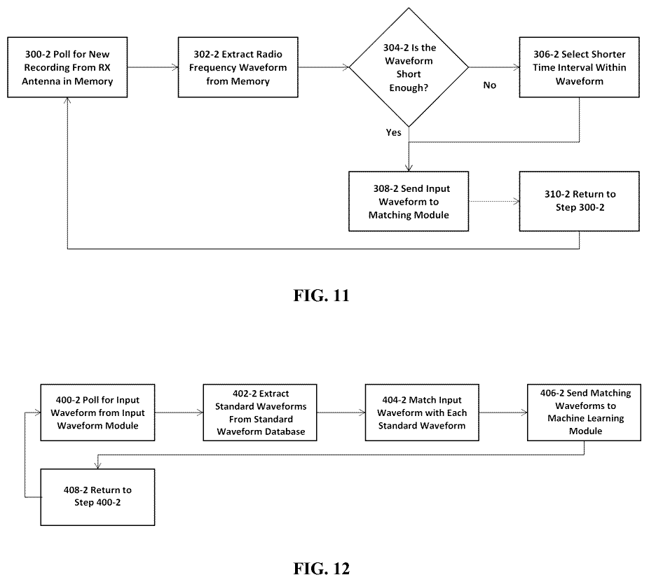

FIG. 11 illustrates an example operation of the input waveform module 126-2. The process may begin with the input waveform module 126-2 polling, at step 300-2, for new recorded data from the one or more RX antennas 111-2 stored in memory 114-2. The input waveform module 126-2 may extract, at step 302-2, the recorded radio frequency waveform from memory. If there is more than one waveform recorded, the input waveform module 126-2 may select each waveform separately and loop through the following steps. The input waveform module 126-2 may determine, at step 304-2, if the waveform is small enough to be an input waveform for the matching module 128-2. This will depend on the computational requirements and/or restrictions of the matching module 128-2. If the waveform is short enough, the input waveform module 126-2 may skip to step 308-2. If the waveform is too long, the input waveform module 126-2 may select, at step 306-2, a shorter time interval within the entire recorded waveform. For example, if the waveform is 5 minutes long, then only a 30 second interval may be selected. The interval may be selected at random or by a selection process. The input waveform module 126-2 may send, at step 308-2, the input waveform to the matching module 128-2. The input waveform module 126-2 may return, at step 310-2, to step 300-2.

FIG. 12 illustrates an example operation of the matching module 128-2. The process may begin with the matching module 128-2 polling, at step 400-2, for an input waveform from the input waveform module 126-2. The matching module 128-2 may extract, at step 402-2, each standard waveform from the standard waveform database 116-2. The matching module 128-2 may match, at step 404-2, the input waveform with each standard waveform. Matching may be determining which standard waveforms the input waveform is similar to. Matching may involve convolution and/or cross-correlation of the waveforms or any other suitable matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time-lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function whose values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-2. The matching module 128-2 may send, at step 406-2, the matching waveforms to the machine learning module 130-2. Matching waveforms may refer to the standard waveforms that were similar to the input waveform, the waveforms that were generated via convolution and/or cross-correlation, or both. The matching module 128-2 may return, at step 408-2, to step 400-2.

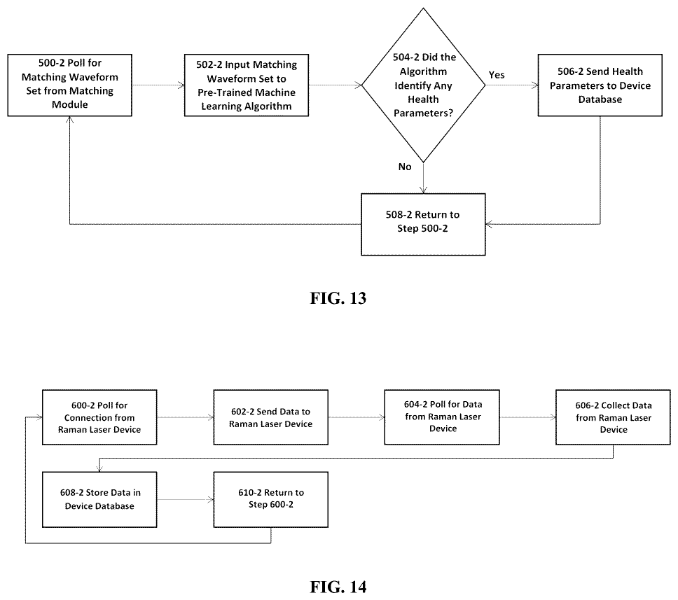

FIG. 13 displays illustrates an example operation of the machine learning module 130-2. The process may begin with the machine learning module 130-2 polling, at step 500-2, for a set of matching waveforms from the matching module 128-2. Matching waveforms may be a set of standard waveforms that are similar to the input waveform or statistical combinations of the input waveform and standard waveforms, such as convolutions or cross-correlations. The machine learning module 130-2 may input, at step 502-2, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During the training process, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-2 may determine, at step 504-2, if the algorithm identified any health parameters. Identification may require a certain interval of confidence. For example, if the machine learning algorithm determines that it is more than 70% likely that a health parameter is correct, that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130-2 may skip to step 508-2. If any health parameters were identified, the machine learning module 130-2 may send, at step 506-2, the health parameters to the device database 132-2. The machine learning module 130-2 may return, at step 508-2, to step 500-2.

FIG. 14 illustrates an example operation of the connection module 134-2. The process may begin with the connection module 134-2 polling, at step 600-2, for a connection from the Raman laser device 144-2. If no connection is detected, the connection module 134-2 may search for a Raman laser device 144-2 that is attempting to connect. The connection module 134-2 may send, at step 602-2, data to the Raman laser device 144-2. This data may be the most recent data from the device 108-2 in the device database 132-2 or directly from the machine learning module 130-2. This data will be used by the Raman laser device 144-2 to set initial measurement conditions and/or assist with matching the measured EMR spectrum with known standard spectrums. The connection module 134-2 may poll, at step 604-2, for data being sent by the connected Raman laser device 144-2. If no connection is detected the connection module 134-2 may search for a Raman laser device 144-2 that is attempting to connect and send data. The connection module 134-2 may collect, at step 606-2, data from the Raman laser device 144-2. This may be real-time data or periodically updated data. The connection module 134-2 may store, at step 608-2, the data from the Raman laser device 144-2 in the device database 132-2. The connection module 134-2 may add contextual data such as the ID of the Raman laser device 144-2, analyte measured by the Raman laser device 144-2, time data was retrieved, etc., to the received data before storage in the device database 132-2. The connection module 134-2 may return, at step 610-2, to step 600-2.

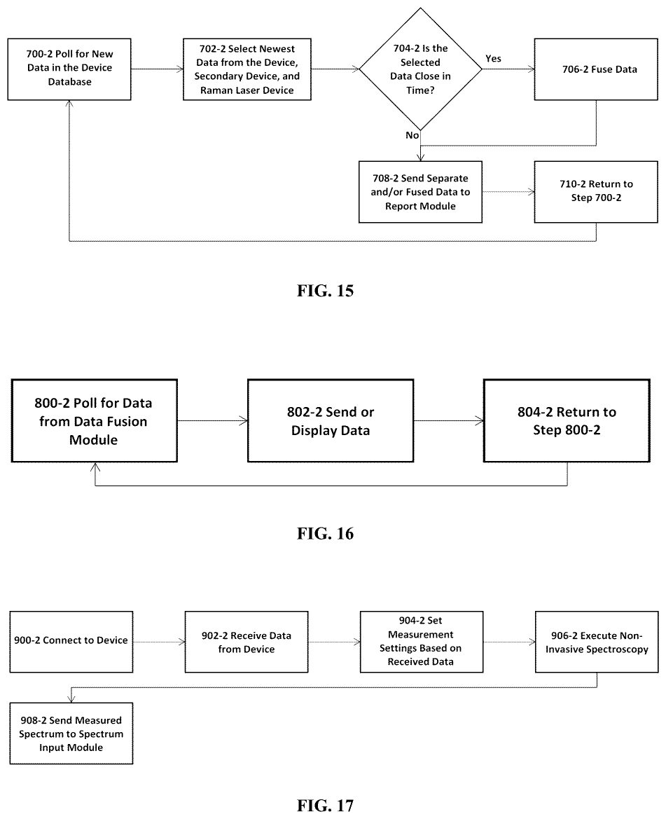

FIG. 15 illustrates an example operation of the data fusion module 136-2. The process may begin with the data fusion module 136-2 polling, at step 700-2, for new data in the device database. This new data may be data from the device 108-2, from the secondary device 140-2, or from the Raman laser device 144-2. The data fusion module 136-2 may select, at step 702-2, the newest data from the device 108-2, the newest data from the secondary device 140-2, and the newest data from the Raman laser device 144-2. This may be a single data point or a set of data. The data fusion module 136-2 may determine, at step 704-2, if the selected data is close in time. Close in time may mean each piece or set of data is timestamped within a few seconds of each other. For example, the newest data from the device 108-2 may be real-time, but the newest reading from the Raman laser device 144-2 may have been 10 minutes ago. This data would not be close in time if the threshold is a few seconds. The threshold may be varied based on the health parameter being measured. For example, if the secondary device 140-2 is recording sleep cycle activity, then recordings from 10 minutes ago may be relevant because of the gradual nature of sleep cycles, but if the Raman laser device 144-2 is recording blood glucose or electrolyte levels, then data from minutes ago may be highly inaccurate. The time threshold for what data is considered close in time may be set by the user of the device, manufacturer of the device, and/or another module. If the selected data is not close in time, the data fusion module may skip to step 708-2. The data fusion module 136-2 may fuse, at step 706-2, the selected data from the device 108-2, secondary device 140-2, and/or Raman laser device 144-2. Fusion of data may describe multiple processes, and which process is performed may depend on the types of data being compared. For example, if both pieces or data sets are measurements of the same parameter, such as glucose, then fusing the data may refer to aligning and combining data to ensure it is accurate and consistent. Aligning may be taking the mean or the median. The data may be enhanced, meaning an error is returned if both measurements are outside a predetermined normal range. For another example, fused data may be a correlation function of two sets of data, such as correlating real-time glucose measurements from the device 108-2 to sleep cycle readings from the secondary device 140-2 in order to track the effect of blood glucose on sleep quality. Fusing the data may refer to adjusting one piece or set of data based on the other. For example, pulse rate readings from the secondary device 140-2 may be used to adjust SPO2 readings from the Raman laser device 144-2. which are calibrated for a normal pulse rate. Fusing the data may refer to generating additional health parameters from the combination of the data. For example, blood glucose measurements from the device 108-2, blood cortisol levels from the Raman laser device 144-2, and heart rate measurements from the secondary device 140-2 can be fused to generate stress or activity level. Fusing data may refer to estimating a risk level based on the data sets. For example, blood glucose data from the device 108-2, heart rate data from the secondary device 140-2, and tissue oxygenation data from the Raman laser device 144-2 may all be factored into a risk score. The data fusion module 136-2 may use statistical algorithms, machine learning, and/or AI to perform the data fusion. The data fusion module 136-2 may send, at step 708-2, each piece or set of data and/or the fused data, if available, to the report module 138-2. The data fusion module 136-2 may return, at step 710-2, to step 700-2.

FIG. 16 illustrates an example operation of the report module 138-2. The process may begin with the report module 138-2 polling, at step 800-2, for data from the data fusion module 136-2. The report module 138-2 may send and/or display, at step 802-2, the received data. If a display is connected to the device 108-2 or can be reached by other means, the report module 138-2 may display the data directly. If not, the report module may send data in real-time or periodically. Data may be sent to an email address, a database, a module, or any other destination. The data may be sent to and/or displayed at multiple locations. The report module 138-2 may return, at step 804-2, to step 800-2.

FIG. 17 illustrates an example operation of the setup module 148-2. The process may begin with the setup module 148-2 connecting, at step 900-2, to the device 108-2 via the RLD comms 146-2. The connection or attempt to connect may be processed by the connection module 134-2. The setup module 148-2 may receive, at step 902-2, data from the device 108-2. This data may be analyte data or other biometric data measured by the device 108-2. For example, the data may be blood glucose level data that indicates the patient's blood glucose level is 120 mg/dL. The setup module 148-2 may set at step 904-2, measurement settings based on the received data. For example, when the glucose level recorded by the device 108-2 is high, only a few wavelengths of EMR may need to be transmitted into the patient in order to confirm the high level of glucose, but when glucose is low, a more robust spectrum of EMR may need to be transmitted in order to get accurate results. The setup module 148-2 may aim to keep power consumption and patient discomfort low by only using the minimal amount of EMR required for the level of accuracy needed. The setup module 148-2 may execute, at step 906-2, a non-invasive spectroscopy by transmitting EMR into the patient's tissue and/or bloodstream and recording the resulting Raman spectrum. The setup module 148-2 may send, at step 908-2, the measured Raman spectrum to the spectrum input module 150-2.

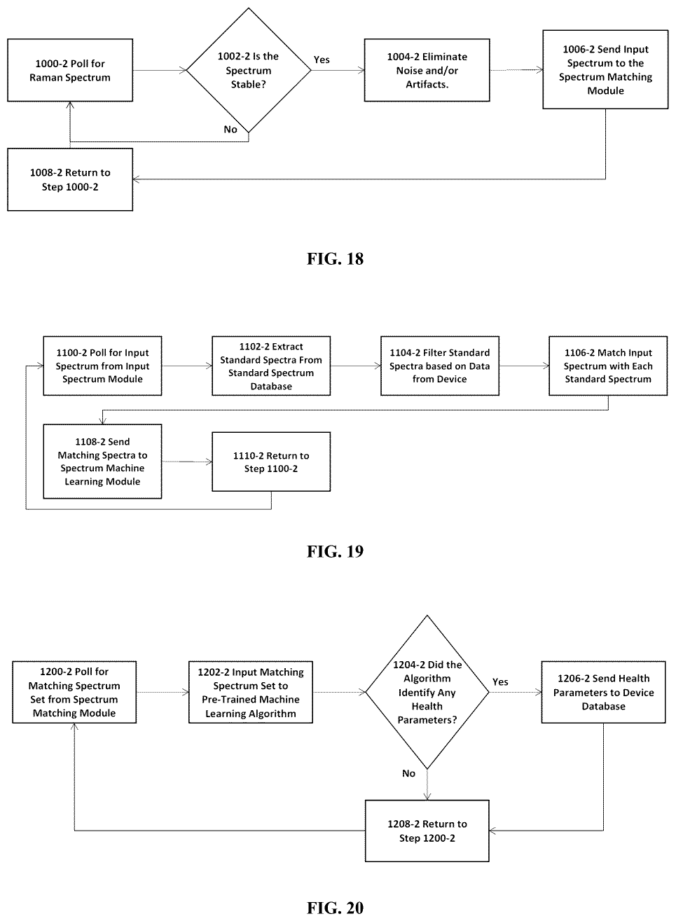

FIG. 18 illustrates an example operation of the input spectrum module 150-2. The process may begin with the input spectrum module 150-2 polling, at step 1000-2, for newly recorded spectrum data from the Raman laser device 144-2. The input spectrum module 150-2 may determine, at step 1002-2, if the recorded Raman spectrum is stable. If the spectrum rapidly changes as recordings are being taken in real-time, then it may not be stable. If the spectrum is unstable, the input spectrum module 150-2 may wait until the spectrum stabilizes. If the spectrum does not stabilize within a set amount of time, then medical staff may be notified that the measurements from the Raman laser device 144-2 are inconclusive and return to step 1000-2. The input spectrum module 150-2 may eliminate, at step 1004-2, noise and/or artifacts from the data. Noise and/or artifacts may be eliminated or reduced using signal averaging, baseline correction, peak fitting, Fourier filtering, spectral smoothing, outlier removal, and interpolation. The input spectrum module 150-2 may send, at step 1006-2, the input spectrum to the spectrum matching module 152-2. The input spectrum module 150-2 may return, at step 1008-2, to step 1000-2.

FIG. 19 illustrates an example operation of the spectrum matching module 152-2. The process may begin with the spectrum matching module 152-2 polling, at step 1100-2, for an input spectrum from the input spectrum module 150-2. The spectrum matching module 152-2 may extract, at step 1102-2, each standard spectrum from the standard spectrum database 156-2. The spectrum matching module 152-2 may filter, at step 1104-2, the standard spectra based on the data from the device 108-2. For example, if the data from the device 108-2 indicates a glucose level of 120 mg/dL and the Raman laser device 144-2 is also detecting glucose levels, then only the standard spectra that are associated with glucose levels in the 80-160 mg/dL range may be used. For another example, if the data from the device 108-2 indicates a glucose level of 80 mg/dL and the Raman laser device 144-2 is detecting another analyte, then only the standard spectra taken from patients who had low glucose, usually due to fasting, may be used. The spectrum matching module 152-2 may match, at step 1106-2, the input spectrum with each standard spectrum. Matching may be determining which standard spectra the input spectrum is similar to. Matching may involve convolution and/or cross-correlation of the spectra. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two spectra, the output will give a similarity value between two spectra, where the highest value represents the most similar pair. Convolution, on the other hand, is a mathematical operation that combines two functions to produce a third function. It is the integral of the product of two functions after one of them is flipped and then shifted. By applying convolution on two spectra, the output will be a function whose values represent the degree of similarity between input signals, where higher values represent more similar signals. These operations may be used in combination and/or with other techniques, such as Fourier transform, to extract information from signals and compare them. Matching spectra may be spectra where the cross-correlation and/or convolution values are close to 1 with respect to time. For example, the threshold value may be 0.85. Any point in the function that results from cross-correlation that is above 0.85 may indicate that the standard spectrum matches the input spectrum. Matching standard spectra, the input spectrum, the cross-correlation of both, and/or the convolution of both may be used as an input to the machine learning algorithm of the spectrum machine learning module 154-2. The spectrum matching module 152-2 may send, at step 1108-2, the matching spectra to the spectrum machine learning module 154-2. Matching spectra may refer to the standard spectra similar to the input spectrum, the spectra generated via convolution and/or cross-correlation, or both. The spectrum matching module 152-2 may return, at step 1110-2, to step 1100-2.

FIG. 20 illustrates an example operation of the spectrum machine learning module 154-2. The process may begin with the spectrum machine learning module 154-2 polling, at step 1200-2, for a set of matching spectra from the spectrum matching module 152-2. Matching spectra may be a set of standard spectra similar to the input spectrum or statistical combinations of the input spectrum and standard spectra, such as convolutions or cross-correlations. The spectrum machine learning module 154-2 may input, at step 1202-2, the set of received spectra into a pre-trained machine learning algorithm. The machine learning algorithm may be trained on similar sets of matched spectra where the input spectrum is from a patient whose health parameters are known. For example, the spectra may be input directly into the algorithm as a set of X and Y values. The matching spectra may each be summarized as a closest fit function or may be transformed into a set of sine waves using a Fourier transform. Training data should be labeled with the correct output, such as the type of spectrum. In order to prepare the data, the spectra need to be processed and converted into a format that can be used by the algorithm. Once the data is prepared, the algorithm is trained on the labeled data. The model uses this data to learn the relationships between the spectra and their corresponding outputs. During the training process, the model will adjust its parameters to minimize the error between its predictions and the correct outputs. Once the model has been trained and fine-tuned, it can be used to recognize spectra in new, unseen data. This could be done by giving the input spectrum, then the algorithm will predict the health parameters. The spectrum machine learning module 154-2 may determine, at step 1204-2, if the algorithm identified any health parameters. Identification may require a certain interval of confidence. For example, if the machine learning algorithm determines that it is more than 70% likely that a health parameter is correct, that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the spectrum machine learning module 154-2 may skip to step 1208-2. If any health parameters were identified, the spectrum machine learning module 154-2 may send, at step 1206-2, the health parameters to the connection module 134-2 via the RLD comms 146-2 to be stored in the device database 132-2. The spectrum machine learning module 154-2 may return, at step 1208-2, to step 1200-2.

In one example implementation of Embodiment 2, a method for measuring one or more analytes using a real-time, non-invasive radio frequency (“RF”) analyte detection device, can include the following: providing a real-time, non-invasive RF analyte detection device; providing a wearable integrated Raman Laser analyte detection device; providing a connection module which connects the real-time, non-invasive RF analyte detection device, and the wearable integrated Raman Laser analyte detection device; storing a first set of data of the one or more analytes obtained from a measurement by the real-time, non-invasive RF analyte detection device; sending the first set of data to the wearable integrated Raman laser analyte detection device; storing a second set of data of the one or more analytes obtained from a measurement by the wearable integrated Raman Laser analyte detection device, wherein measurement settings of the wearable integrated Raman laser analyte detection device and data analysis run on the second set of data is influenced by the first set of data; executing a fusion module to form fused data from the first set of data and the second set of data; and reporting the fused data results.

### Embodiment 3

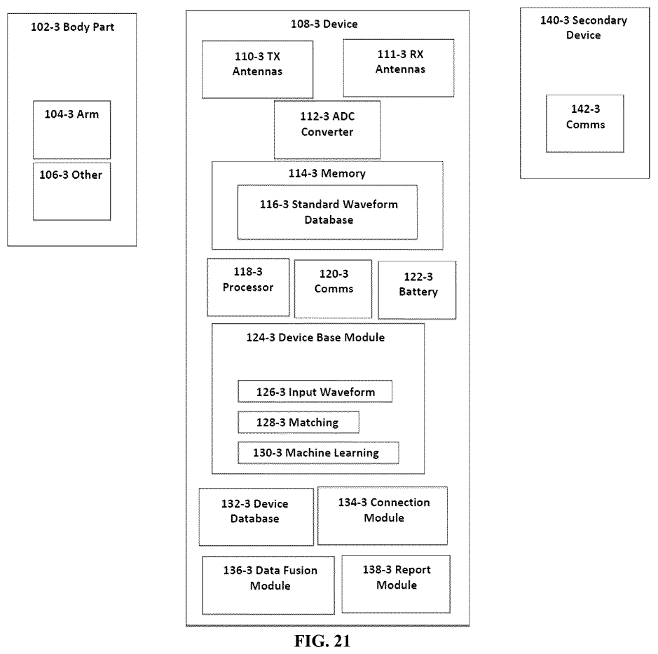

FIG. 21 is a schematic illustration of a system for radio frequency health monitoring. This system is configured to be attached or in proximity to a body part 102-3. The body part 102-3 may be an arm 104-3. The body part 102-3 may be another body part besides an arm, such as a leg, finger, chest, head, or any other body part from which useful medical parameters can be taken. The system may comprise a device 108-3, which may be a wearable and portable device such as, but not limited to, a cell phone, a smartwatch, a tracker, a wearable monitor, a wristband, and a personal blood monitoring device. The system may further comprise one or more transmit (“TX”) antennas 110-3. The one or more TX antennas may be configured to transmit RF in the RF Activated Range from 500 MHZ to 300 GHZ. A pre-defined frequency may correspond to a range suitable for the human body in one embodiment. For example, the one or more TX antennas would transmit radio frequency signals at a range of 120-126 GHz. The system may further comprise one or more receive (“RX”) antennas 111-3. The one or more RX antennas may be configured to receive the RF signals in response to the RF signal transmitted from the one or more TX antennas. The system may further comprise an ADC converter 112-3, which may be configured to convert the received RF signals from an analog signal into a digital processor readable format. The system may further comprise memory 114-3, which may be configured to store the transmitted RF signals by the one or more TX antennas 110-3 and store the received portion of the response or responded RF signals from the one or more RX antennas 111-3. Further, the memory 114-3 may also store the converted digital processor readable format by the ADC converter 112-3. The memory 114-3 may include suitable logic, circuitry, and/or interfaces that may be configured to store a machine code and/or a computer program with at least one code section executable by the processor 118-3. Examples of implementation of the memory 114-3 may include, but are not limited to, Random Access Memory (RAM), Read Only Memory (ROM), Hard Disk Drive (HDD), and/or a Secure Digital (SD) card. The system may further comprise a standard waveform database 116-3, which may contain standard waveforms for known patterns. These may be raw or converted device readings from patients or persons with known conditions. For example, the standard waveform database 116-3 may include raw or converted device readings from the right arm of a patient known to have diabetes or an average of multiple patients. This data can be compared to readings from a person with an unknown condition in order to determine if the waveforms taken from that person match any of the known standard waveforms. The system may further comprise a processor 118-3, which may facilitate the operation of the device 108-3 according to the instructions stored in the memory 114-3. The processor 118-3 may include suitable logic, circuitry, interfaces, and/or code that may be configured to execute a set of instructions stored in the memory 114-3. The system may further comprise comms 120-3, which may communicate with a network. Examples of networks may include, but are not limited to, the Internet, a cloud network, a Wireless Fidelity (Wi-Fi) network, a Wireless Local Area Network (WLAN), a Local Area Network (LAN), Long Term Evolution (LTE), and/or a Metropolitan Area Network (MAN). The system may further comprise a battery 122-3, which may power hardware modules of the device 108-3. The device 108-3 may be configured with a charging port to recharge the battery 122-3. Charging of the battery 122-3 may be wired or wireless. The system may further comprise a device base module 124-3, which may be configured to store instructions for executing the computer program from the converted digital processor readable format of the ADC converter 112-3. The device base module 124-3 may be configured to facilitate the operation of the processor 118-3, the memory 114-3, the one or more TX antennas 110-3, the one or more RX antennas 111-3, and the comms 120-3. Further, the device base module 124-3 may be configured to create polling of the RF Activated Range from 500 MHZ to 300 GHZ. It can be noted that the device base module 124-3 may be configured to filter the RF Activated Range from 500 MHZ to 300 GHZ received from one or more RX antennas 111-3. The system may further comprise an input waveform module 126-3, which may extract a radio frequency waveform from memory. This may be the raw or converted recording of data from the one or more RX antennas 111-3 from a patient wearing the device. If the entire radio frequency is too long for effective matching, the input waveform module 126-3 may select a time interval within the data set. This input waveform may then be sent to the matching module 128-3. The system may further comprise a matching module 128-3, which may match the input waveform and each of the standard waveforms in the standard waveform database 116-3 by performing a convolution and/or cross-correlation of the input waveform and the standard waveform. These convolutions and/or cross-correlations are then sent to the machine learning module 130-3. The system may further comprise a machine learning module 130-3 which has been trained to identify health parameters based on the convolution and/or cross-correlations of the input and standard waveforms. The machine learning module 130-3 receives the convolutions and cross-correlations from the matching module 128-3 and outputs any health parameters identified. The system may further comprise a device database 132-3, which may store the health parameters output by the machine learning module 130-3. The system may further comprise a connection module 134-3, which may connect to the secondary device 140-3 and collect data which then may be stored in the device database 132-3. The system may further comprise a data fusion module 136-3, which may fuse the real-time analyte data from the device 108-3 and collected data from the secondary device 140-3 stored in the device database 132-3. Fusing data may refer to the process of combining multiple sources of information or data sets into a single integrated output. This process involves aligning and combining data from different sources while ensuring it remains accurate and consistent. For example, fusing data from the non-invasive RF analyte detection device and other physiological devices, such as heart rate, sleep system, or pulse rate devices, would enable the identification of correlations or patterns that may exist between the analyte levels and other physiological variables. Such information can be useful in providing a more comprehensive understanding of the individual's health status and may aid in the development of targeted interventions or treatments. The system may further comprise a report module 138-3, which may display a report of the fused data from the data fusion module 136-3. This report may be sent to an email address, displayed on a screen, sent to another module, or otherwise reported. The system may further comprise a secondary device 140-3, which may be a heart rate device, sleep system device, pulse rate device, or sensor inserted under the skin to measure glucose level device or any other device that produces data which may be collected by the connection module 134-3. The system may further comprise the comms 142-3, which may be a communication element such as a Wi-Fi or RFC transmitter that can send data from the secondary device 140-3 to the connection module 134-3 of the device 108-3.

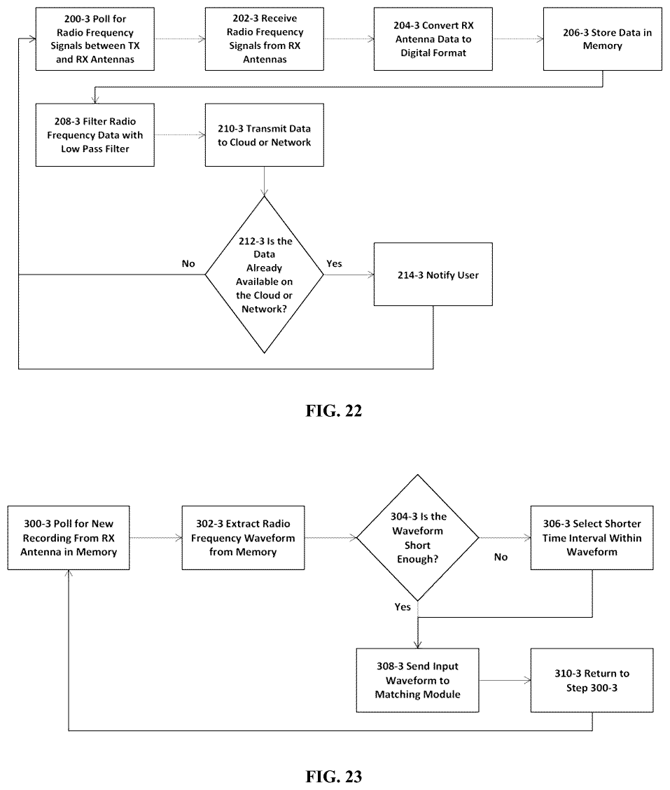

FIG. 22 illustrates an example operation of the device base module 124-3. The process may begin with the device base module 124-3 polling the Active Range RF signals between the one or more TX antennas 110-3 and the one or more RX antennas 111-3 at step 200-3. The device base module 124-3 may be configured to read and process instructions stored in the memory 114-3 using the processor 118-3. The one or more TX antennas 110-3 may be configured to transmit RF in the RF Activated Range from 500 MHZ to 300 GHZ. For example, the one or more TX antennas 110-3 may use RF signals at a range of 500 MHZ to 300 GHZ. The device base module 124-3 may receive the RF frequency signals from the one or more RX antennas 111-3 at step 202-3. For example, one or more RX antennas 111-3 receives a response or responded RF of frequency range 300-330 GHz from the patient's blood vessels. The device base module 124-3 may be configured to convert the received RF signals into a digital format using the ADC 112-3 at step 204-3. For example, the received RF of 300-330 GHz frequency range is converted into a 10-bit data signal. The device base module 124-3 may be configured to store converted digital format into the memory 114-3 at step 206-3. The device base module 124-3 may be configured to filter the stored RF signals at step 208-3. The device base module 124-3 may be configured to filter each RF signal using the low pass filter. For example, the device base module 124-3 filters the RF of frequency range 300-330 GHz to the RF of frequency range 300-310 GHz. The device base module 124-3 may be configured to transmit the filtered RF signals to the cloud or other network using the comms module 120-3 at step 210-3. For example, the device base module 124-3 may be configured to transmit data about the RF in the RF Activated Range from 500 MHZ to 300 GHZ to the cloud. The device base module 124-3 may be configured to determine whether the transmitted data is already available in the cloud or other network at step 212-3. The device base module 124-3, using the comms 120-3, communicates with the cloud network to determine that data about the transmitted RF signal is already available. The device base module 124-3 may determine that the transmitted data is not already present in the cloud. The device base module 124-3 may be redirected back to step 202-3 to poll the RF signals between the one or more TX antennas 110-3 and the one or more RX antennas 111-3. For example, the device base module 124-3 determines that the transmitted RF in the RF Activated Range from 500 MHZ to 300 GHZ is not present in the cloud, and corresponding to the transmitted signal, there is no data related to the blood glucose level of the patient. The device base module 124-3 may determine that transmitted data is already present in the cloud. For example, the device base module 124-3 reads cloud notification of the patient's blood glucose level as 110 mg/dL corresponding to an RF in the RF Activated Range from 500 MHZ to 300 GHZ. The device base module 124-3 may continue to step 214-3. The device base module 124-3 may notify the user via the device 108-3 of health information, for example, blood glucose level.

FIG. 23 illustrates an example operation of the input waveform module 126-3. The process may begin with the input waveform module 126-3 polling, at step 300-3, for newly recorded data from the one or more RX antennas 111-3 stored in memory 114-3. The input waveform module 126-3 may extract, at step 302-3, the recorded radio frequency waveform from memory. If there is more than one waveform recorded, the input waveform module 126-3 may select each waveform separately and loop through the following steps. The input waveform module 126-3 may determine, at step 304-3, if the waveform is small enough to be an input waveform for the matching module 128-3. This will depend on the computational requirements and/or restrictions of the matching module 128-3. If the waveform is short enough, the input waveform module 126-3 may skip to step 308-3. If the waveform is too long, the input waveform module 126-3 may select, at step 306-3, a shorter time interval within the entire recorded waveform. For example, if the waveform is 5 minutes long, then only a 30-second interval may be selected. The interval may be selected at random or by a selection process. The input waveform module 126-3 may send, at step 308-3, the input waveform to the matching module 128-3. The input waveform module 126-3 may return, at step 310-3, to step 300-3.

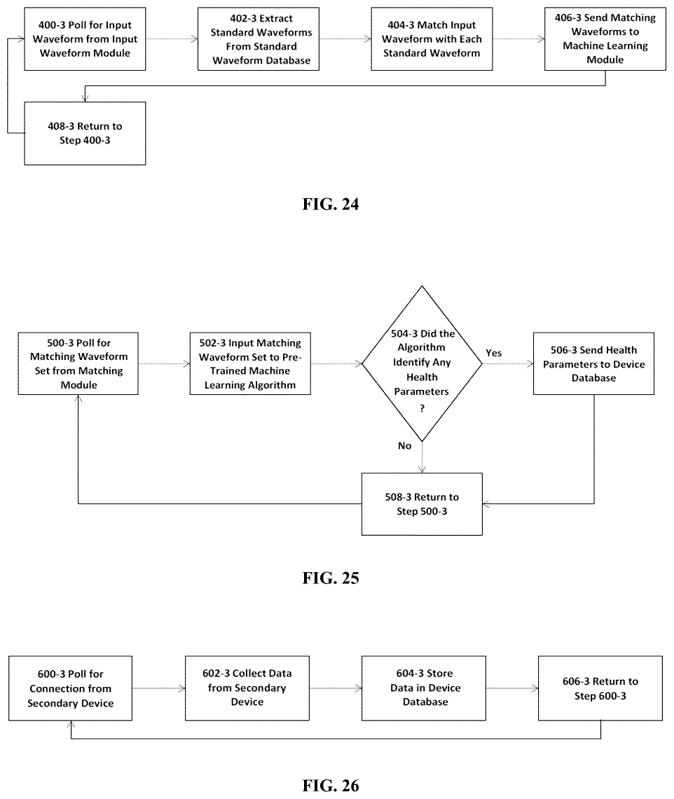

FIG. 24 illustrates an example operation of the matching module 128-3. The process may begin with the matching module 128-3 polling, at step 400-3, for an input waveform from the input waveform module 126-3. The matching module 128-3 may extract, at step 402-3, each standard waveform from the standard waveform database 116-3. The matching module 128-3 may match, at step 404-3, the input waveform with each standard waveform. Matching may be determining which standard waveforms the input waveform is similar to. Matching may involve convolution and/or cross-correlation of the waveforms. Cross-correlation and convolution are mathematical operations that can be used to determine the similarity between two wave functions. They are often used in signal processing and image recognition applications to find patterns or features in data. Cross-correlation measures the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function in which values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-3. The matching module 128-3 may send, at step 406-3, the matching waveforms to the machine learning module 130-3. Matching waveforms may refer to the standard waveforms similar to the input waveform, the waveforms generated via convolution and/or cross-correlation, or both. The matching module 128-3 may return, at step 408-3, to step 400-3.

FIG. 25 illustrates an example operation of the machine learning module 130-3. The process may begin with the machine learning module 130-3 polling, at step 500-3, for a set of matching waveforms from the matching module 128-3. Matching waveforms may be a set of standard waveforms that are similar to the input waveform or statistical combinations of the input waveform and standard waveforms, such as convolutions or cross-correlations. The machine learning module 130-3 may input, at step 502-3, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During training, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-3 may determine, at step 504-3, if the algorithm identified any health parameters. Identification may require a certain interval of confidence. For example, if the machine learning algorithm determines that it is more than 70% likely that a health parameter is correct, that parameter may be considered identified. If there are multiple conflicting parameters, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130-3 may skip to step 508-3. If any health parameters were identified, the machine learning module 130-3 may send, at step 506-3, the health parameters to the device database 132-3. The machine learning module 130-3 may return, at step 508-3, to step 500-3.

FIG. 26 illustrates an example operation of the connection module 134-3. The process may begin with the connection module 134-3 polling, at step 600-3, for a connection from the secondary device 140-3. If no connection is detected, the connection module 134-3 may search for a secondary device that is attempting to connect. The connection module 134-3 may collect, at step 602-3, data from the secondary device. This may be real-time data or periodically updated data. The connection module 134-3 may store, at step 604-3, the data from the secondary device 140-3 in the device database 132-3. The connection module 134-3 may add contextual data such as the ID of the secondary device, type of secondary device, time data was retrieved, etc., to the received data before storage in the device database 132-3. The connection module 134-3 may return, at step 606-3, to step 600-3.

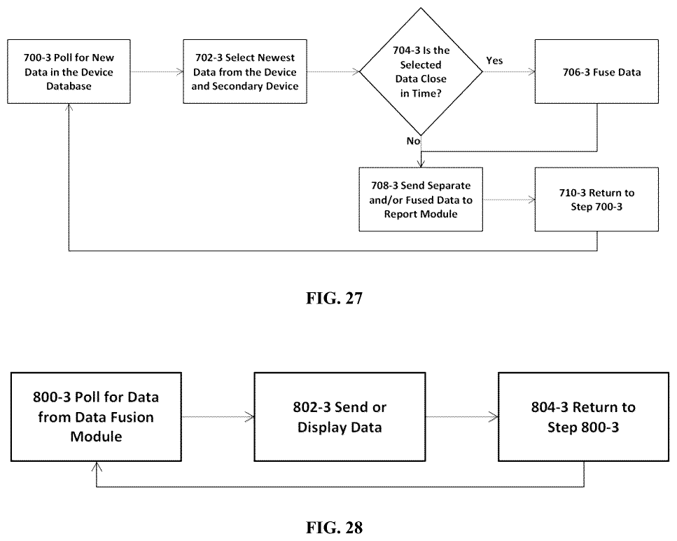

FIG. 27 illustrates an example operation of the data fusion module 136-3. The process may begin with the data fusion module 136-3 polling, at step 700-3, for new data in the device database. This new data may be data from the device 108-3 or from the secondary device 140-3. The data fusion module 136-3 may select, at step 702-3, the newest data from the device 108-3 and the newest data from the secondary device 140-3. This may be a single data point or a set of data. The data fusion module 136-3 may determine, at step 704-3, if the selected data is close in time. Close in time may mean each piece or set of data is timestamped within a few seconds of each other. For example, the newest data from the device 108-3 may be real-time, but the newest reading from the secondary device may have been 10 minutes ago. This data will not be close in time if the threshold is a few seconds. The threshold may be varied based on the health parameter being measured. For example, if the secondary device is recording sleep cycle activity, then recordings from 10 minutes ago may be relevant because of the gradual nature of sleep cycles, but if the secondary device is recording heart rate, then data from minutes ago may be highly inaccurate. The time threshold for what data is considered close in time may be set by the device user, manufacturer of the device, and/or another module. If the selected data is not close in time, the data fusion module may skip to step 708-3. The data fusion module 136-3 may fuse, at step 706-3, the selected data from the device 108-3 and secondary device 140-3. The fusion of data may describe multiple processes and which process is performed depending on the data types being compared. For example, if both pieces or data sets are measurements of the same parameter, such as glucose, then fusing the data may refer to aligning and combining data to ensure it is accurate and consistent. Aligning may be taking the mean or the median. The data may be enhanced, meaning an error is returned if both measurements are outside a predetermined normal range. For another example, fused data may be a correlation function of two sets of data, such as correlating real-time glucose measurements from the device 108-3 to sleep cycle readings from the secondary device 140-3 in order to track the effect of blood glucose on sleep quality. Fusing the data may refer to adjusting one piece or set of data based on the other. For example, pulse rate readings from the secondary device 140-3 may be used to adjust SP02 readings from the device 108-3, which are calibrated for a normal pulse rate. Fusing the data may refer to generating additional health parameters from the combination of the data. For example, cortisol measurements from the device 108-3 and heart rate measurements from the secondary device 140-3 can be fused to generate stress or activity level. The data fusion module 136-3 may use statistical algorithms, machine learning, and/or AI to perform the data fusion. The data fusion module 136-3 may send, at step 708-3, each piece or set of data and/or the fused data, if available, to the report module 138-3. The data fusion module 136-3 may return, at step 710-3, to step 700-3.

FIG. 28 illustrates an example operation of the report module 138-3. The process may begin with the report module 138-3 polling, at step 800-3, for data from the data fusion module 136-3. The report module 138-3 may send and/or display, at step 802-3, the received data. If a display is connected to the device 108-3 or can be reached by other means, the report module 138-3 may display the data directly. If not, the report module may send data in real-time or periodically. Data may be sent to an email address, a database, a module, or any other destination. The data may be sent to and/or displayed at multiple locations. The report module 138-3 may return, at step 804-3, to step 800-3.

In one example implementation of Embodiment 3, a method for measuring one or more analytes using a real-time, non-invasive radio frequency (“RF”) analyte detection device can include the following: providing the real-time, non-invasive RF analyte detection device; providing a connection module to at least one other device; storing a first set of data of the one or more analytes obtained from a measurement by the real-time, non-invasive RF analyte detection device; storing a second set of data obtained from a connection module; executing a fusion module to combine the first set of data obtained from the real-time, non-invasive RF analyte detection device and the second set of data obtained from the connection module to form fused data; and reporting the fused data results.

### Embodiment 4

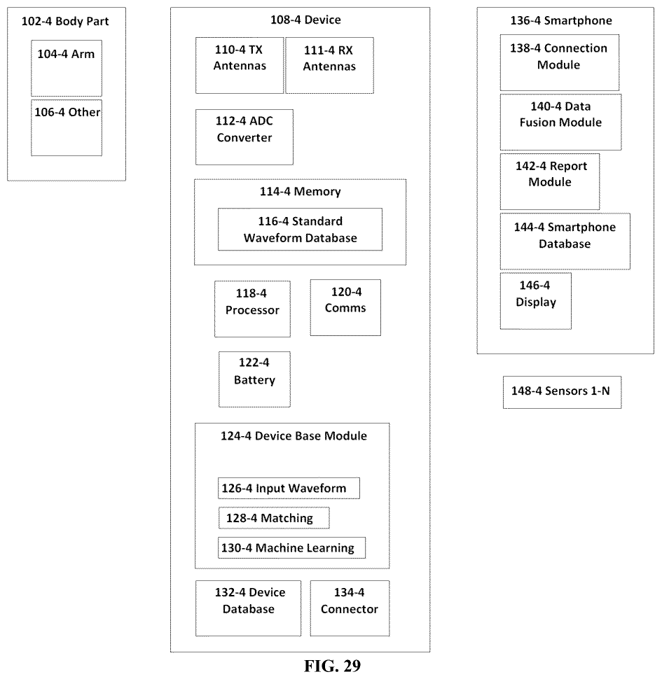

FIG. 29 is a schematic illustration of a system for radio frequency health monitoring. The system is configured to be attached to a body part 102-4. The body part 102-4 may be an arm 104-4. The body part 102-4 may be another body part besides an arm, such as a leg, finger, chest, head, or any other body part from which useful medical parameters can be taken. The system may comprise a device 108-4, which may be a wearable and portable device such as, but not limited to, a cell phone, a smartwatch, a tracker, a wearable monitor, a wristband, and a personal blood monitoring device. The system may further comprise one or more transmit (“TX”) antennas 110-4. The one or more TX antennas 110-4 may be configured to transmit RF in the RF Activated Range from 500 MHZ to 300 GHZ. A pre-defined frequency may correspond to a range suitable for the human body in one embodiment. For example, the one or more TX antennas 110-4 would transmit use radio frequency signals at a range of 120-126 GHz. The system may further comprise one or more receive (“RX”) antennas 111-4. The one or more RX antennas 111-4 may be configured to receive the response or responded RF signals in response to the RF signal from the one or more TX antennas 110-4. The system may further comprise an ADC converter 112-4, which may be configured to convert the responded RF signals from the one or more RX antennas 111-4 from an analog signal into a digital processor readable format. The system may further comprise memory 114-4, which may be configured to store data related to the transmitted RF signals by the one or more TX antennas 110-4 and receive a portion of the transmitted RF signals from the one or more RX antennas 111-4. Further, the memory 114-4 may also store the converted digital processor readable format by the ADC converter 112-4. The memory 114-4 may include suitable logic, circuitry, and/or interfaces that may be configured to store a machine code and/or a computer program with at least one code section executable by the processor 118-4. Examples of implementation of the memory 114-4 may include, but are not limited to, Random Access Memory (RAM), Read Only Memory (ROM), Hard Disk Drive (HDD), and/or a Secure Digital (SD) card. The system may further comprise a standard waveform database 116-4, which may contain standard waveforms for known patterns. These may be raw or converted device readings from patients or persons with known conditions. For example, the standard waveform database 116-4 may include raw or converted device readings from the right arm of a patient known to have diabetes or an average of multiple patients. This data can be compared to readings from a person with an unknown condition in order to determine if the waveforms taken from that person match any of the known standard waveforms. The system may further comprise the processor 118-4, which may facilitate the operation of the device 108-4 according to the instructions stored in the memory 114-4. The processor 118-4 may include suitable logic, circuitry, interfaces, and/or code that may be configured to execute a set of instructions stored in the memory 114-4. The system may further comprise comms 120-4, which may communicate with a network. Examples of networks may include, but are not limited to, the Internet, a cloud network, a Wireless Fidelity (Wi-Fi) network, a Wireless Local Area Network (WLAN), a Local Area Network (LAN), Long Term Evolution (LTE), and/or a Metropolitan Area Network (MAN). The system may further comprise a battery 122-4, which may power hardware modules of the device 108-4. The device 108-4 may be configured with a charging port to recharge the battery 122-4. Charging of the battery 122-4 may be wired or wireless. The system may further comprise a device base module 124-4, which may be configured to store instructions for executing the computer program from the converted digital processor readable format of the ADC converter 112-4. The device base module 124-4 may be configured to facilitate the operation of the processor 118-4, the memory 114-4, the one or more TX antennas 110-4, the one or more RX antennas 111-4, and the comms 120-4. Further, the device base module 124-4 may be configured to create polling of the RF Activated Range from 500 MHZ to 300 GHZ. It can be noted that the device base module 124-4 may be configured to filter the RF Activated Range from 500 MHZ to 300 GHZ received from one or more RX antennas 111-4. The system may further comprise an input waveform module 126-4, which may extract a radio frequency waveform from memory 114-4. This may be the raw or converted data recording from the one or more RX antennas 111-4 from a patient wearing the device. If the entire radio frequency is too long for effective matching, the input waveform module 126-4 may select a time interval within the data set. This input waveform may then be sent to the matching module 128-4. The system may further comprise the matching module 128-4, which may match the input waveform and each of the standard waveforms in the standard waveform database 116-4 by performing a convolution and/or cross-correlation of the input waveform and the standard waveform. These convolutions and/or cross-correlations are then sent to the machine learning module. The system may further comprise a machine learning module 130-4 which has been trained to identify health parameters based on the convolution and/or cross-correlations of the input and standard waveforms. The machine learning module 130-4 receives the convolutions and cross-correlations from the matching module 128-4 and outputs any health parameters identified. The system may further comprise a device database 132-4, which may store the health parameters output by the machine learning module 130-4. The system may further comprise a connector 134-4, which may be a physical connection element such as a USB-C cord from the device 108-4 to the smartphone 136-4. The connector 134-4 may be used in place of or in conjunction with comms 120-4 to deliver data. The system may further comprise or be configured to connect to a smartphone 136-4, an electronic device comprising a central processing unit (CPU), memory, a display, a user interface, and one or more communication interfaces configured to enable wireless communication with other devices and networks. The smartphone 136-4 may include a touch-sensitive display screen for displaying information, images, and video and receiving user inputs through finger or stylus touch. The smartphone 136-4 may comprise one or more sensors, such as a camera, accelerometer, gyroscope, GPS, and/or biometric sensor, for capturing, processing, and analyzing data. The smartphone 136-4 may also include various hardware and software components for enabling wireless communication, such as a cellular modem, Wi-Fi, Bluetooth, and/or NFC, as well as one or more antennas for transmitting and receiving signals. The smartphone 136-4 may be configured to run various applications for communication, productivity, entertainment, and gaming, which can be downloaded and installed from an app store or other source. The system may further comprise a connection module 138-4, which may connect to the device 108-4 and the sensors 148-4 and collect data which then may be stored in the smartphone database 144-4. The system may further comprise a data fusion module 140-4, which may fuse the real-time analyte data from the device 108-4 and collected data from the sensors 148-4 stored in the device database 132-4. Fusing data may refer to the process of combining multiple sources of information or data sets into a single integrated output. This process involves aligning and combining data from different sources while ensuring it remains accurate and consistent. For example, fusing data from the non-invasive RF analyte detection device and other physiological devices, such as heart rate, sleep system, or pulse rate devices, would enable the identification of correlations or patterns that may exist between the analyte levels and other physiological variables. Such information can be useful in providing a more comprehensive understanding of the individual's health status and may aid in the development of targeted interventions or treatments. The system may further comprise a report module 142-4, which may display a report of the fused data from the data fusion module 140-4. This report may be viewed on the smartphone 136-4 via the display 146-4. The system may further comprise a smartphone database 144-4, which may store data retrieved from the device 108-4 and the sensors 148-4. The system may further comprise the display 146-4, which may provide visual feedback to the user of the smartphone 136-4. The display may be a flat screen made of thin, lightweight material, such as glass or plastic, highly transparent and resistant to scratches and impacts. The display 146-4 may be of various types, such as LCD (liquid crystal display), OLED (organic light-emitting diode), AMOLED (active-matrix organic light-emitting diode), or IPS (in-plane switching). The system may further comprise one or more sensors 148-4, which may detect physiological parameters of the body such as temperature, heart rate, movement, etc., and relay that information to the smartphone 136-4. Examples of sensors may include: accelerometers which measure acceleration and can be used to track physical activity, such as steps taken, distance traveled, and calories burned; heart rate monitors which measure the heart rate and can be used to track exercise intensity and monitor heart health; electrocardiogram (ECG) sensors which measure the electrical activity of the heart and can be used to detect abnormal heart rhythms and other cardiac conditions; electrodermal activity (EDA) sensors which measure the skin's electrical conductance and can be used to track stress levels and emotional responses; temperature sensors which measure body temperature and can be used to monitor fever and track changes in body temperature over time; glucose sensors which measure glucose levels in sweat, tears, or saliva and can be used to monitor diabetes and adjust insulin therapy; oxygen saturation sensors which measure the level of oxygen in the blood and can be used to monitor respiratory function and detect hypoxemia; sleep trackers which measure sleep duration and quality and can be used to track sleep patterns and detect sleep disorders; and posture sensors which measure body posture and can be used to monitor sitting or standing positions to promote proper alignment and prevent back pain.

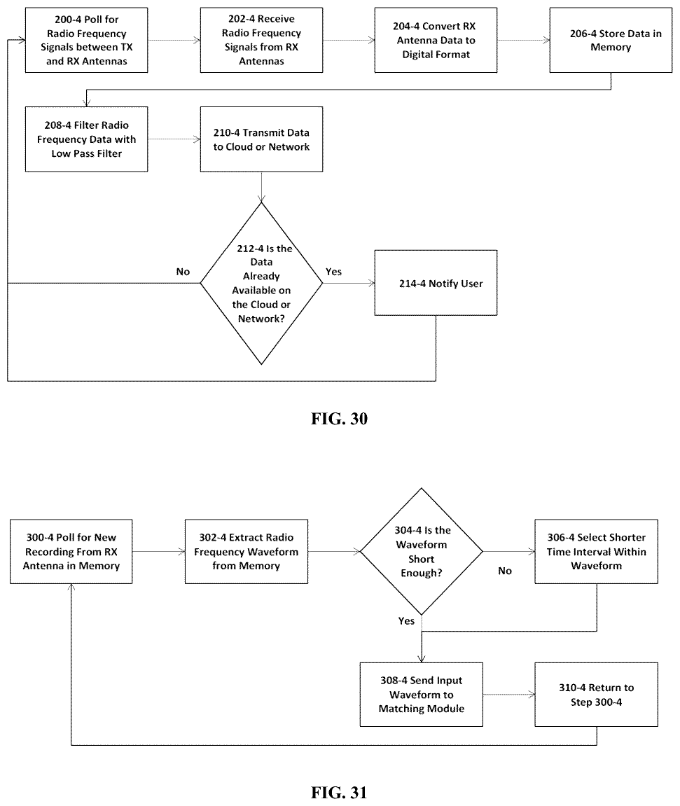

FIG. 30 illustrates an example operation of the device base module 124-4. The process may begin with the device base module 124-4 polling the Active Range RF signals between the one or more TX antennas 110-4 and the one or more RX antennas 111-4 at step 200-4. The device base module 124-4 may be configured to read and process instructions stored in the memory 114-4 using the processor 118-4. The one or more TX antennas 110-4 may be configured to transmit RF in the RF Activated Range from 500 MHZ to 300 GHZ. For example, the one or more TX antennas 110-4 may use RF signals at a range of 500 MHZ to 300 GHZ. The device base module 124-4 may receive the response or responded RF frequency signals from the one or more RX antennas 111-4 at step 202-4. For example, one or more of the RX antennas 111-4 receives a response or responded RF signal of frequency range 300-330 GHz from the patient's blood vessels. The device base module 124-4 may be configured to convert the received RF signals into a digital format using the ADC 112-4 at step 204-4. For example, the received RF of 300-330 GHz frequency range is converted into a 10-bit data signal. The device base module 124-4 may be configured to store converted digital format into the memory 114-4 at step 206-4. The device base module 124-4 may be configured to filter the stored RF signals at step 208-4. The device base module 124-4 may be configured to filter each RF signal using the low pass filter. For example, the device base module 124-4 filters the RF of 300-330 GHz to the RF of 300-310 GHz. The device base module 124-4 may be configured to transmit the filtered RF signals to the cloud or other network using the comms module 120-4 at step 210-4. For example, the device base module 124-4 may be configured to transmit RF in the RF Activated Range from 500 MHZ to 300 GHZ to the cloud. The device base module 124-4 may be configured to determine whether the transmitted data is already available in the cloud or other network at step 212-4. The device base module 124-4, using the comms 120-4, communicates with the cloud network to determine that the transmitted RF signal is already available. The device base module 124-4 may determine that the transmitted data is not already present in the cloud. The device base module 124-4 may be redirected back to step 200-4 to poll the RF signals between the one or more TX antennas 110-4 and the one or more RX antennas 111-4. For example, the device base module 124-4 determines that data related to the transmitted RF in the RF Activated Range from 500 MHZ to 300 GHZ is not present in the cloud, and corresponding to the transmitted signal, there is no data related to the blood glucose level of the patient. The device base module 124-4 may determine that transmitted data is already present in the cloud. For example, the device base module 124-4 reads cloud notification of the patient's blood glucose level as 110-4 mg/dL corresponding to an RF signal in the RF Activated Range from 500 MHZ to 300 GHZ. The device base module 124-4 may continue to step 214-4. The device base module 124-4 may notify the user via the device 108-4 of health information, for example, blood glucose level.

FIG. 31 illustrates an example operation of the input waveform module 126-4. The process may begin with the input waveform module 126-4 polling, at step 300-4, for newly recorded data from the one or more RX antennas 111-4 stored in memory 114-4. The input waveform module 126-4 may extract, at step 302-4, the recorded radio frequency waveform from memory 114-4. If there is more than one waveform recorded, the input waveform module 126-4 may select each waveform separately and loop through the following steps. The input waveform module 126-4 may determine, at step 304-4, if the waveform is small enough to be an input waveform for the matching module 128-4. This will depend on the computational requirements and/or restrictions of the matching module 128-4. If the waveform is short enough, the input waveform module 126-4 may skip to step 308-4. If the waveform is too long, the input waveform module 126-4 may select, at step 306-4, a shorter time interval within the entire recorded waveform. For example, if the waveform is 5 minutes long, then only a 30-second interval may be selected. The interval may be selected at random or by a selection process. The input waveform module 126-4 may send, at step 308-4, the input waveform to the matching module 128-4. The input waveform module 126-4 may return, at step 310-4, to step 300-4.

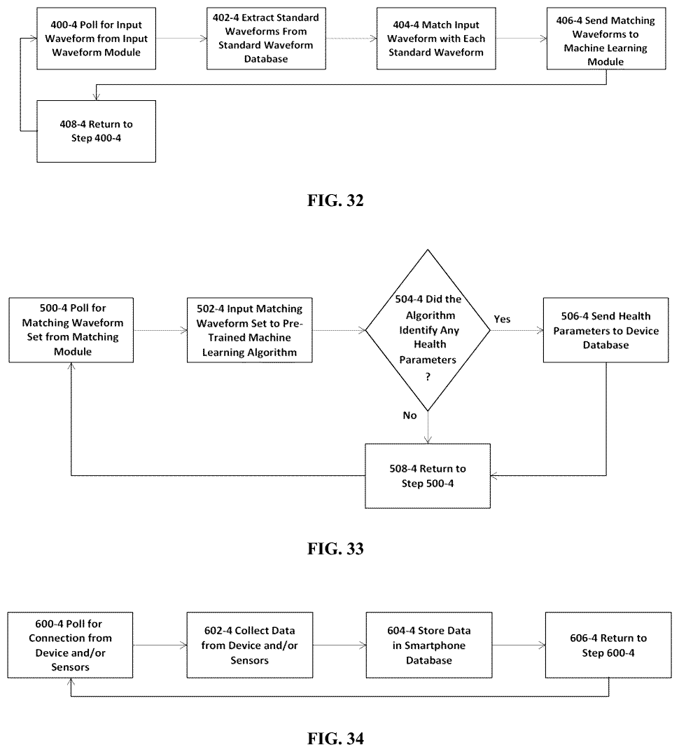

FIG. 32 illustrates an example operation of the matching module 128-4. The process may begin with the matching module 128-4 polling, at step 400-4, for an input waveform from the input waveform module 126-4. The matching module 128-4 may extract, at step 402-4, each standard waveform from the standard waveform database 116-4. The matching module 128-4 may match, at step 404-4, the input waveform with each standard waveform. Matching may be determining which standard waveforms the input waveform is similar to. Matching may involve convolution and/or cross-correlation of the waveforms. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function in which values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-4. The matching module 128-4 may send, at step 406-4, the matching waveforms to the machine learning module 130-4. Matching waveforms may refer to the standard waveforms similar to the input waveform, the waveforms generated via convolution and/or cross-correlation, or both. The matching module 128-4 may return, at step 408-4, to step 400-4.

FIG. 33 illustrates an example operation of the machine learning module 130-4. The process may begin with the machine learning module 130-4 polling, at step 500-4, for a set of matching waveforms from the matching module 128-4. Matching waveforms may be a set of standard waveforms similar to the input waveform or statistical combinations of the input waveform and standard waveforms, such as convolutions or cross-correlations. The machine learning module 130-4 may input, at step 502-4, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During training, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-4 may determine, at step 504-4, if the algorithm identified any health parameters. Identification may require a certain interval of confidence. For example, if the machine learning algorithm determines that it is more than 70% likely that a health parameter is correct, that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130-4 may skip to step 508-4. If any health parameters were identified, the machine learning module 130-4 may send, at step 506-4, the health parameters to the device database 132-4. The machine learning module 130-4 may return, at step 508-4, to step 500-4.

FIG. 34 illustrates an example operation of the connection module 138-4 of the smartphone 136-4. The process may begin with the connection module 138-4 polling, at step 600-4, for a connection from the device 108-4 and/or sensors 148-4. If no connection is detected, the connection module 138-4 may search for a device 108-4 or sensor 148-4 that is attempting to connect. The connection module 138-4 may collect, at step 602-4, data from the device 108-4 and/or sensors 148-4. This may be real-time data or periodically updated data. Examples of sensors may include: accelerometers which measure acceleration and can be used to track physical activity, such as steps taken, distance traveled, and calories burned; heart rate monitors which measure the heart rate and can be used to track exercise intensity and monitor heart health; electrocardiogram (ECG) sensors which measure the electrical activity of the heart and can be used to detect abnormal heart rhythms and other cardiac conditions; electrodermal activity (EDA) sensors which measure the skin's electrical conductance and can be used to track stress levels and emotional responses; temperature sensors which measure body temperature and can be used to monitor fever and track changes in body temperature over time; glucose sensors which measure glucose levels in sweat, tears, or saliva and can be used to monitor diabetes and adjust insulin therapy; oxygen saturation sensors which measure the level of oxygen in the blood and can be used to monitor respiratory function and detect hypoxemia; sleep trackers which measure sleep duration and quality and can be used to track sleep patterns and detect sleep disorders; and posture sensors which measure body posture and can be used to monitor sitting or standing positions to promote proper alignment and prevent back pain. The connection module 138-4 may store, at step 604-4, the data from the device 108-4 and/or sensors 148-4 in the smartphone database 144-4. The connection module 138-4 may add contextual data such as the ID of the device 108-4 and/or sensors 148-4, type of device 108-4 and/or sensors 148-4, time data was retrieved, etc., to the received data before storage in the smartphone database 144-4. The connection module 138-4 may return, at step 606-4, to step 600-4.

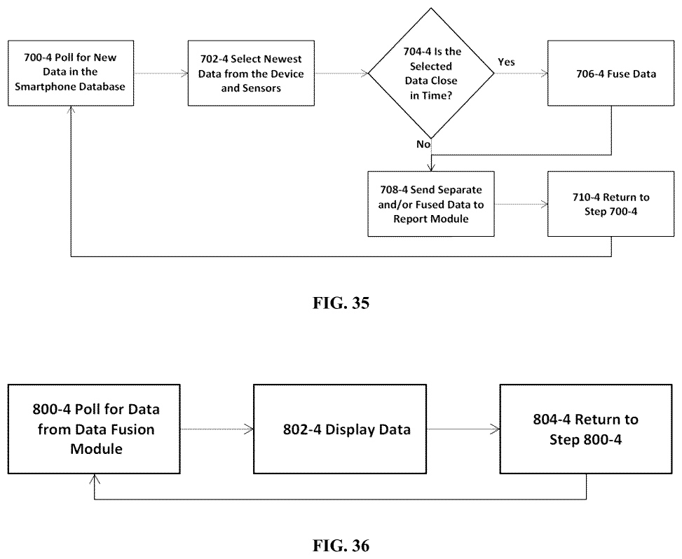

FIG. 35 illustrates an example operation of the data fusion module 140-4 of the smartphone 136-4. The process may begin with the data fusion module 140-4 polling, at step 700-4, for new data in the smartphone database 144-4. This new data may be data from the device 108-4 or sensors 148-4. The data fusion module 140-4 may select, at step 702-4, the newest data from the device 108-4 and the newest data from each of the sensors 148-4. This may be a single data point or a set of data. The data fusion module 140-4 may determine, at step 704-4, if the selected data from the device 108-4 and at least one sensor 148-4 are close in time. Close in time may mean each piece or set of data is timestamped within a few seconds of each other. For example, the newest data from the device 108-4 may be real-time, but the newest reading from a sensor 148-4 may have been 10 minutes ago. This data would not be close in time if the threshold is a few seconds. The threshold may be varied based on the health parameter being measured. For example, if a sensor 148-4 is recording sleep cycle activity, then recordings from 10 minutes ago may be relevant because of the gradual nature of sleep cycles, but if a sensor 148-4 is recording heart rate, then data from minutes ago may be highly inaccurate. The time threshold for what data is considered close in time may be set by the user of the device, manufacturer of the device, and/or another module. If none of the selected data is close in time, the data fusion module may skip to step 708-4. The data fusion module 140-4 may fuse, at step 706-4, the selected data from the device 108-4 and sensors 148-4. Data from sensors 148-4 that is not close in time to the data from the device 108-4 may not be used in data fusion. The fusion of data may describe multiple processes and which process is performed depending on the data types being compared. For example, if both pieces or data sets are measurements of the same parameter, such as glucose, then fusing the data may refer to aligning and combining data to ensure it is accurate and consistent. Aligning may be taking the mean or the median. The data may be enhanced, meaning an error is returned if both measurements are outside a predetermined normal range. This type of data fusion may help eliminate false positives and/or false negatives and provide a more comprehensive picture of a person's glucose levels. For another example, fused data may be a correlation function of two sets of data, such as correlating real-time glucose measurements from the device 108-4 to sleep cycle readings from the sensors 148-4 in order to track the effect of blood glucose on sleep quality. Fusing the data may refer to adjusting one piece or set of data based on the other. For example, pulse rate readings from the sensors 148-4 may be used to adjust SP02 readings from the device 108-4, which are calibrated for a normal pulse rate. Fusing the data may refer to generating additional health parameters from the combination of the data. For example, cortisol measurements from the device 108-4 and heart rate measurements from the sensors 148-4 can be fused to generate stress or activity level. The data fusion module 140-4 may use statistical algorithms, machine learning, and/or AI to perform the data fusion. The data fusion module 140-4 may send, at step 708-4, each piece or set of data and/or the fused data, if available, to the report module 142-4. The data fusion module 140-4 may return, at step 710-4, to step 700-4.

FIG. 36 illustrates an example operation of the report module 142-4 of the smartphone 136-4. The process may begin with the report module 142-4 polling, at step 800-4, for data from the data fusion module 140-4. The report module 142-4 may display, at step 802-4, the received data via the display 146-4. The report module 142-4 may also send data in real-time or periodically. Data may be sent to an email address, a database, a module, or any other destination. The data may be sent to and/or displayed at multiple locations. The user may use this data to assist with diseases or illnesses. For example, in an embodiment a user may be diabetic. Glucose readings are taken from the user by both the device 108-4 and a sensor 148-4, which detects glucose in sweat. The results are fused such that the number of false spikes and drops in glucose due to an issue with the device 108-4 or sensor 148-4 are reduced or eliminated entirely. When the user views a display of their glucose level over time, they may be able to view each recording and the fused data to see where the false spikes and drops would have been if there was no data fusion. For another example, a user may have hypertension, and their blood pressure is monitored using both a device 108-4 and a smartphone app. The wearable device measures blood pressure continuously throughout the day, while the app prompts users to measure their blood pressure at regular intervals. The results from both sources are fused using a machine learning algorithm, which considers factors such as activity level, sleep quality, and diet. The fused data provides a more accurate and comprehensive picture of the user's blood pressure over time, allowing for better management of hypertension and the prevention of complications. For another example, a user may have asthma, and their lung function is monitored using a spirometer, a pulse oximeter, and a device 108-4 measuring blood oxygen level. The spirometer measures lung function by having the user blow into a mouthpiece, while the pulse oximeter measures the oxygen saturation in the user's blood. The results from all three devices are fused using a data fusion algorithm, which takes into account factors such as the user's age, gender, and smoking history. The fused data provides a more accurate and comprehensive picture of the user's lung function over time, allowing for better management of asthma and the prevention of exacerbations. The report module 142-4 may return, at step 804-4, to step 800-4.

In one example implementation of Embodiment 4, a method for measuring one or more analytes using a real-time, non-invasive radio frequency (“RF”) analyte detection device can include the following: providing the real-time, non-invasive RF analyte detection device on a user; providing a sensor on the user; obtaining a first data from the real-time, non-invasive RF analyte detection device on the one or more analytes in the user; retrieving the first data from the real-time, non-invasive RF analyte detection device and the second data from the sensor; executing a fusion module to combine the first data obtained from the real-time, non-invasive RF analyte detection device and the second data from the sensor to form a fused data; and reporting the fused data results.

### Embodiment 5

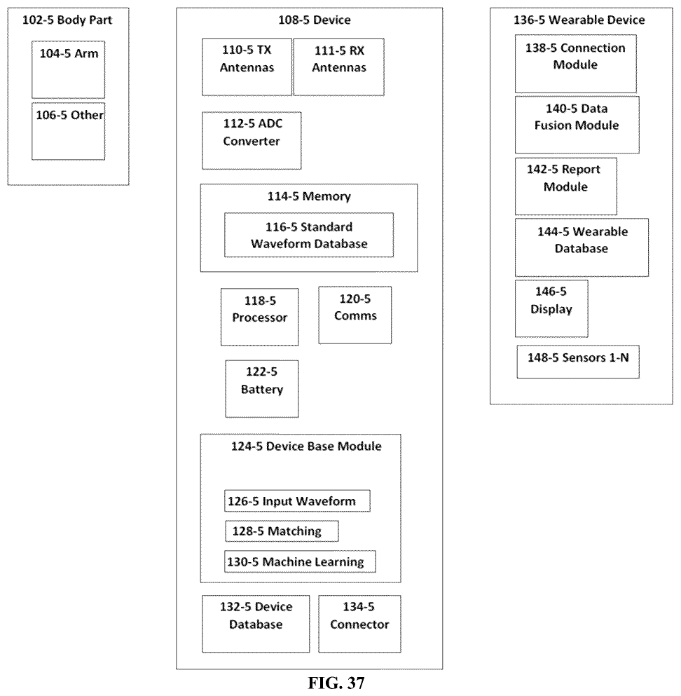

FIG. 37 is a schematic example of a system for radio frequency health monitoring, according to an embodiment. The system may comprise a device 108-5 that is configured to be attached or in proximity to a body part 102-5. The body part 102-5 may be an arm 104-5. The body part 102-5 may be another body part besides an arm, such as a leg, finger, chest, head, or any other body part from which useful medical parameters can be taken. The device 108-5 may be a wearable and portable device such as, but not limited to, a cell phone, a smartwatch, a tracker, a wearable monitor, a wristband, and a personal blood monitoring device. The system may further comprise one or more transmit (“TX”) antennas 110-5. The one or more TX antennas 110-5 may be configured to transmit RF in the RF Activated Range from 500 MHZ to 300 GHZ. In one embodiment, a pre-defined frequency may correspond to a range suitable for the human body. For example, the one or more TX antennas 110-5 may use radio frequency signals at a range of 120-126 GHz. The system may further comprise one or more RX antennas 111-5. The one or more RX antennas 111-5 may be configured to receive the RF signals in response to the RF signal(s) from the one or more TX antennas 110-5. The system may further comprise an ADC converter 112-5, which may be configured to convert the RF signals from an analog signal into a digital processor readable format. The system may further comprise memory 114-5, which may be configured to store data related to the transmitted RF signals by the one or more TX antennas 110-5 and receive a portion of the transmitted RF signals from the one or more RX antennas 111-5. Further, the memory 114-5 may also store the converted digital processor readable format by the ADC converter 112-5. The memory 114-5 may include suitable logic, circuitry, and/or interfaces that may be configured to store a machine code and/or a computer program with at least one code section executable by the processor 118-5. Examples of implementation of the memory 114-5 may include, but are not limited to, Random Access Memory (RAM), Read Only Memory (ROM), Hard Disk Drive (HDD), and/or a Secure Digital (SD) card. The system may further comprise a standard waveform database 116-5, which may contain standard waveforms for known patterns. These may be raw or converted device readings from user or persons with known conditions. For example, the standard waveform database 116-5 may include raw or converted device readings from the right arm of a user known to have diabetes or an average of multiple users or patients. This data can be compared to readings from a person with an unknown condition in order to determine if the waveforms taken from that person match any of the known standard waveforms. The system may further comprise the processor 118-5, which may facilitate the operation of the device 108-5 according to the instructions stored in the memory 114-5. The processor 118-5 may include suitable logic, circuitry, interfaces, and/or code that may be configured to execute a set of instructions stored in the memory 114-5. The system may further comprise comms 120-5, which may communicate with a network. Examples of networks may include, but are not limited to, the Internet, a cloud network, a Wireless Fidelity (Wi-Fi) network, a Wireless Local Area Network (WLAN), a Local Area Network (LAN), Long Term Evolution (LTE), and/or a Metropolitan Area Network (MAN). The system may further comprise a battery 122-5, which may power hardware modules of the device 108-5. The device 108-5 may be configured with a charging port to recharge the battery 122-5. Charging of the battery 122-5 may be wired or wireless. The system may further comprise a device base module 124-5, which may be configured to store instructions for executing the computer program related to the converted digital processor readable format of the ADC converter 112-5. The device base module 124-5 may be configured to facilitate the operation of the processor 118-5, the memory 114-5, the one or more TX antennas 110-5, the one or more RX antennas 111-5, and the comms 120-5. Further, the device base module 124-5 may be configured to create polling of the RF Activated Range from 500 MHZ to 300 GHZ. It can be noted that the device base module 124-5 may be configured to filter the RF Activated Range from 500 MHZ to 300 GHZ received from one or more RX antennas 111-5. The system may further comprise an input waveform module 126-5, which may extract a radio frequency waveform from memory. This may be the raw or converted data recording from the one or more RX antennas 111-5 from a user wearing the device. If the entire radio frequency is too long for effective matching, the input waveform module 126-5 may select a time interval within the data set. This input waveform may then be sent to the matching module 128-5. The system may further comprise the matching module 128-5, which may match the input waveform and each of the standard waveforms in the standard waveform database 116-5 by performing a convolution and/or cross-correlation of the input waveform and the standard waveform. These convolutions and/or cross-correlations are then sent to the machine learning module 130-5. The system may further comprise the machine learning module 130-5 which has been trained to identify health parameters based on the convolution and/or cross-correlations of the input and standard waveforms. The machine learning module 130-5 receives the convolutions and cross-correlations from the matching module 128-5 and outputs any health parameters identified. The system may further comprise a device database 132-5, which may store the health parameters output by the machine learning module 130-5. The system may further comprise a connector 134-5, which may be a physical connection element such as a USB-C cord from the device 108-5 to the wearable device 136-5. The connector 134-5 may be used in place of or in conjunction with comms 120-5 to deliver data. The system may further comprise a wearable device 136-5 which may be a wearable electronic device comprising a central processing unit (CPU), memory, a display, a user interface, and one or more communication interfaces configured to enable wireless communication with other devices and networks. The wearable device 136-5 may include a touch-sensitive display screen for displaying information, images, and video and receiving user inputs through finger or stylus touch. The wearable device 136-5 may comprise one or more sensors, such as a camera, accelerometer, gyroscope, GPS, and/or biometric sensor, for capturing, processing, and analyzing data. The wearable device 136-5 may also include various hardware and software components for enabling wireless communication, such as a cellular modem, Wi-Fi, Bluetooth, and/or NFC, as well as one or more antennas for transmitting and receiving signals. The wearable device 136-5 may be configured to run various applications for communication, productivity, entertainment, and gaming, which can be downloaded and installed from an app store or other source. The system may further comprise a connection module 138-5, which may connect to the device 108-5 and the sensors 148-5 and collect data which then may be stored in the wearable device database 144-5. The system may further comprise a data fusion module 140-5, which may fuse the real-time analyte data from device 108-5 and collect data from sensors 148-5 in the wearable database 144-5. Fusing data may refer to the process of combining multiple sources of information or data sets into a single integrated output. This process involves aligning and combining data from different sources while ensuring that it remains accurate and consistent. For example, fusing data from the non-invasive RF analyte detection device and other physiological devices, such as heart rate, sleep system, or pulse rate devices, would enable the identification of correlations or patterns that may exist between the analyte levels and other physiological variables. Such information can be useful in providing a more comprehensive understanding of the individual's health status and may aid in the development of targeted interventions or treatments. The system may further comprise a report module 142-5, which may display a report of the fused data from the data fusion module 140-5. This report may be viewed on the wearable device 136-5 via the display 146-5. The system may further comprise a wearable device database 144-5, which may store data retrieved from the device 108-5 and the sensors 148-5. The system may further comprise a display 146-5, which may provide visual feedback to the user of the wearable device 136-5. The display may be a flat screen made of thin, lightweight material, such as glass or plastic, highly transparent and resistant to scratches and impacts. The display 146-5 may be of various types, such as LCD (liquid crystal display), OLED (organic light-emitting diode), AMOLED (active-matrix organic light-emitting diode), or IPS (in-plane switching). The system may further comprise one or more sensors 148-5, which may detect physiological parameters of the body such as temperature, heart rate, movement, etc., and store that information in the wearable database 144-5. Examples of sensors may include: accelerometers which measure acceleration and can be used to track physical activity, such as steps taken, distance traveled, and calories burned; heart rate monitors which measure the heart rate and can be used to track exercise intensity and monitor heart health; electrocardiogram (ECG) sensors which measure the electrical activity of the heart and can be used to detect abnormal heart rhythms and other cardiac conditions; electrodermal activity (EDA) sensors which measure the skin's electrical conductance and can be used to track stress levels and emotional responses; temperature sensors which measure body temperature and can be used to monitor fever and track changes in body temperature over time; glucose sensors which measure glucose levels in sweat, tears, or saliva and can be used to monitor diabetes and adjust insulin therapy; oxygen saturation sensors which measure the level of oxygen in the blood and can be used to monitor respiratory function and detect hypoxemia; sleep trackers which measure sleep duration and quality and can be used to track sleep patterns and detect sleep disorders; and posture sensors which measure body posture and can be used to monitor sitting or standing positions to promote proper alignment and prevent back pain.

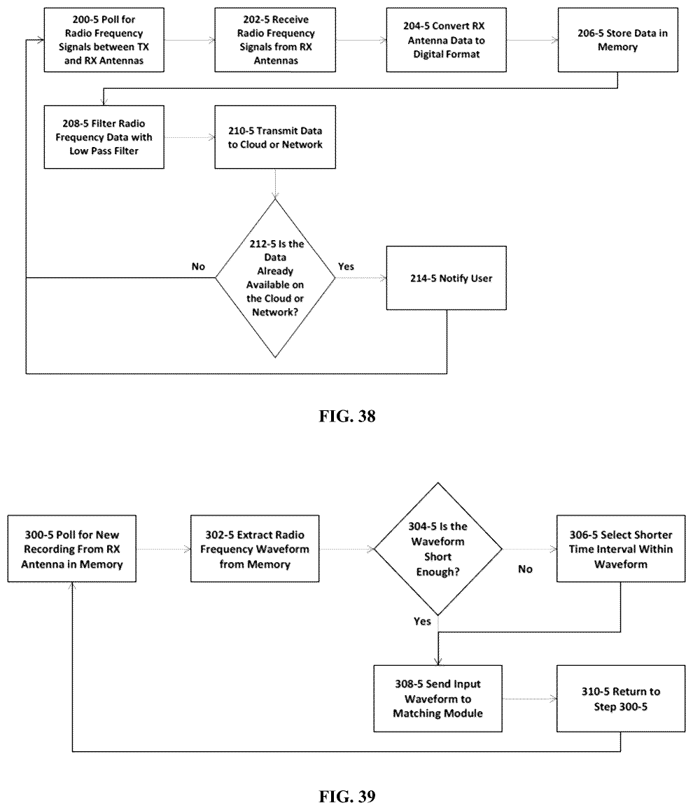

FIG. 38 illustrates an example operation of the device base module 124-5. The process may begin with the device base module 124-5 polling the Active Range RF signals between the one or more TX antennas 110-5 and the one or more RX antennas 111-5 at step 200-5. The device base module 124-5 may be configured to read and process instructions stored in the memory 114-5 using the processor 118-5. The one or more TX antennas 110-5 may be configured to transmit RF in the RF Activated Range from 500 MHZ to 300 GHZ. For example, the one or more TX antennas 110-5 may use RF signals at a range of 500 MHZ to 300 GHZ. The device base module 124-5 may receive the data related to the RF frequency signals from the one or more RX antennas 111-5 at step 202-5. For example, an RX antenna 111-5 may receive an RF of frequency range 300-330 GHz from the user's blood vessels. The device base module 124-5 may be configured to convert the received RF signals into a digital format using the ADC 112-5 at step 204-5. For example, the received RF of frequency range 300-330 GHz is converted into a 10-bit data signal. The device base module 124-5 may be configured to store converted digital format into the memory 114-5 at step 206-5. The device base module 124-5 may be configured to filter the stored RF signals at step 208-5. The device base module 124-5 may be configured to filter each RF signal using the low pass filter. For example, the device base module 124-5 filters the RF of frequency range 300-330 GHz to the RF of frequency range 300-310 GHz The device base module 124-5 may be configured to transmit the filtered RF signals to the cloud or other network using the comms module 120-5 at step 210-5. For example, the device base module 124-5 may be configured to transmit RF in the RF Activated Range from 500 MHZ to 300 GHZ to the cloud. The device base module 124-5 may be configured to determine whether the transmitted data is already available in the cloud or other network at step 212-5. The device base module 124-5, using the comms 120-5, communicates with the cloud network to determine that the transmitted RF signal is already available. The device base module 124-5 may determine that the transmitted data is not already present in the cloud. The device base module 124-5 may be redirected back to step 200-5 to poll the RF signals between the one or more TX antennas 110-5 and the one or more RX antennas 111-5. For example, the device base module 124-5 determines that the transmitted RF in the RF Activated Range from 500 MHZ to 300 GHZ is not present in the cloud, and corresponding to the transmitted signal, there is no data related to the blood glucose level of the user. The device base module 124-5 may determine that transmitted data is already present in the cloud. For example, the device base module 124-5 reads cloud notification of the user's blood glucose level as 110 mg/dL corresponding to an RF in the RF Activated Range from 500 MHZ to 300 GHZ. The device base module 124-5 may continue to step 214-5. The device base module 124-5 may notify the user via the device 108-5 of health information, for example, blood glucose level.

FIG. 39 illustrates an example operation of the input waveform module 126-5. The process may begin with the input waveform module 126-5 polling, at step 300-5, for newly recorded data from the one or more RX antennas 111-5 stored in memory 114-5. The input waveform module 126-5 may extract, at step 302-5, the recorded radio frequency waveform from memory. If there is more than one waveform recorded, the input waveform module 126-5 may select each waveform separately and loop through the following steps. The input waveform module 126-5 may determine, at step 304-5, if the waveform is small enough to be an input waveform for the matching module 128-5. This will depend on the computational requirements and/or restrictions of the matching module 128-5. If the waveform is short enough, the input waveform module 126-5 may skip to step 308-5. If the waveform is too long, the input waveform module 126-5 may select, at step 306-5, a shorter time interval within the entire recorded waveform. For example, if the waveform is 5 minutes long, then only a 30-second interval may be selected. The interval may be selected at random or by a selection process. The input waveform module 126-5 may send, at step 308-5, the input waveform to the matching module 128-5. The input waveform module 126-5 may return, at step 310-5, to step 300-5.

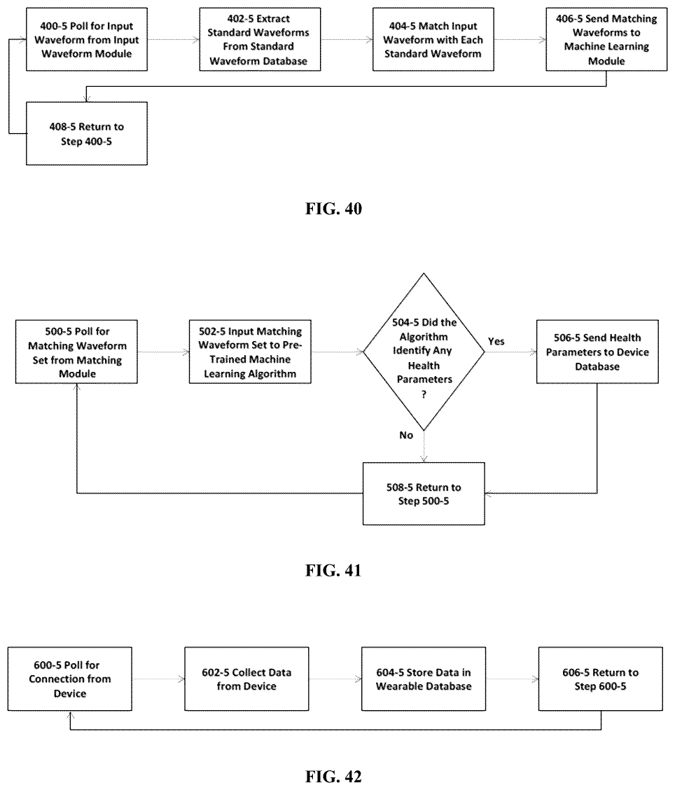

FIG. 40 illustrates an example operation of the matching module 128-5. The process may begin with the matching module 128-5 polling, at step 400-5, for an input waveform from the input waveform module 126-5. The matching module 128-5 may extract, at step 402-5, each standard waveform from the standard waveform database 116-5. The matching module 128-5 may match, at step 404-5, the input waveform with each standard waveform. Matching may be determining which standard waveforms the input waveform is similar to. Matching may involve convolution and/or cross-correlation of the waveforms. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function in which values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-5. The matching module 128-5 may send, at step 406-5, the matching waveforms to the machine learning module 130-5. Matching waveforms may refer to the standard waveforms similar to the input waveform, the waveforms generated via convolution and/or cross-correlation, or both. The matching module 128-5 may return, at step 408-5, to step 400-5.

FIG. 41 illustrates an example operation of the machine learning module 130-5. The process may begin with the machine learning module 130-5 polling, at step 500-5, for a set of matching waveforms from the matching module 128-5. Matching waveforms may be a set of standard waveforms similar to the input waveform or statistical combinations of the input waveform and standard waveforms, such as convolutions or cross-correlations. The machine learning module 130-5 may input, at step 502-5, the set of received waveforms into a pre-trained machine learning algorithm. The machine learning algorithm may be trained on similar sets of matched waveforms where the input waveform is from a user or patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During training, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-5 may determine, at step 504-5, if the algorithm identified any health parameters. Identification may require a certain interval of confidence. For example, if the machine learning algorithm determines that a health parameter is more than 70% likely correct, that parameter may be considered identified. If multiple conflicting parameters exist, then the most confident may be used. For example, if the algorithm determines that it is 75% likely that the user's blood glucose level is between 110-115 mg/dL and 90% likely that the user's blood glucose is between 105-110 mg/dL, then the more confident value of 105-110 mg/dL may be identified. If none of the results from the machine learning algorithm are above the confidence threshold, or the results are otherwise inconclusive, the machine learning module 130-5 may skip to step 508-5. If any health parameters were identified, the machine learning module 130-5 may send, at step 506-5, the health parameters to the device database 132-5. The machine learning module 130-5 may return, at step 508-5, to step 500-5.

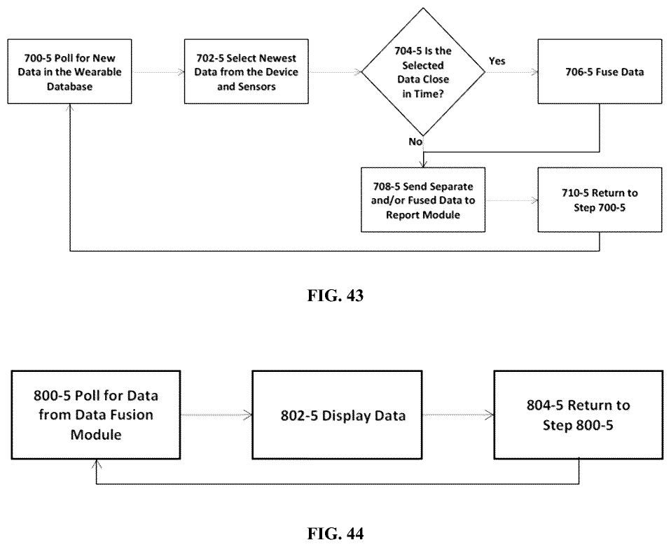

FIG. 42 illustrates an example operation of the connection module 138-5 of the wearable device 136-5. The process may begin with the connection module 138-5 polling, at step 600-5, for a connection from the device 108-5. If no connection is detected, the connection module 138-5 may search for a device 108-5 that is attempting to connect. The connection module 138-5 may collect, at step 602-5, data from the device 108-5. This may be real-time data or periodically updated data. The connection module 138-5 may store, at step 604-5, the data from the device 108-5 in the wearable database 144-5. The connection module 138-5 may add contextual data such as the ID of the device 108-5, type of device 108-5, time data was retrieved, etc., to the received data before storage in the wearable database 144-5. The connection module 138-5 may return, at step 606-5, to step 600-5.

FIG. 43 illustrates an example operation of the data fusion module 140-5. The process may begin with the data fusion module 140-5 polling, at step 700-5, for new data in the wearable database 144-5. This new data may be data from the device 108-5 or sensors 148-5. The data fusion module 140-5 may select, at step 702-5, the newest data from the device 108-5 and the newest data from each of the sensors 148-5. This may be a single data point or a set of data. The data fusion module 140-5 may determine, at step 704-5, if the selected data from the device 108-5 and at least one sensor 148-5 is close in time. Close in time may mean each piece or set of data is timestamped within a few seconds of each other. For example, the newest data from the device 108-5 may be real-time, but the newest reading from a sensor 148-5 may have been 10 minutes ago. This data would not be close in time if the threshold is a few seconds. The threshold may be varied based on the health parameter being measured. For example, if a sensor 148-5 is recording sleep cycle activity, then recordings from 10 minutes ago may be relevant because of the gradual nature of sleep cycles, but if a sensor 148-5 is recording heart rate, then data from minutes ago may be highly inaccurate. The time threshold for what data is considered close in time may be set by the user of the device, manufacturer of the device, and/or another module. If none of the selected data is close in time, the data fusion module may skip to step 708-5. The data fusion module 140-5 may fuse, at step 706-5, the selected data from the device 108-5 and sensors 148-5. Data from sensors 148-5 that are not close in time to the data from the device 108-5 may not be used in data fusion. The fusion of data may describe multiple processes and which process is performed depending on the data types being compared. For example, if both pieces or data sets are measurements of the same parameter, such as glucose, then fusing the data may refer to aligning and combining data to ensure it is accurate and consistent. Aligning may be taking the mean or the median. The data may be enhanced, meaning an error is returned if both measurements are outside a predetermined normal range. This type of data fusion may help eliminate false positives and/or false negatives and provide a more comprehensive picture of a person's glucose levels. For another example, fused data may be a correlation function of two sets of data, such as correlating real-time glucose measurements from the device 108-5 to sleep cycle readings from the sensors 148-5 in order to track the effect of blood glucose on sleep quality. Fusing the data may refer to adjusting one piece or set of data based on the other. For example, pulse rate readings from the sensors 148-5 may be used to adjust SP02 readings from the device 108-5, which are calibrated for a normal pulse rate. Fusing the data may refer to generating additional health parameters from the combination of the data. For example, cortisol measurements from the device 108-5 and heart rate measurements from the sensors 148-5 can be fused to generate stress or activity level. The data fusion module 140-5 may use statistical algorithms, machine learning, and/or AI to perform the data fusion. For example, a learning module may be trained using a Support Vector Regression (SVR) algorithm. The SVR algorithm is a machine learning technique that can be trained on input variables, such as skin temperature readings, and used to predict a continuous output variable, such as glucose levels, with high accuracy. The SVR algorithm may use a kernel function to map input variables into a higher-dimensional feature space, where a linear regression model is applied to estimate the output variable. The algorithm may be optimized through a training process that involves adjusting the kernel function and other parameters to minimize a cost function, such as the mean squared error. The data fusion module 140-5 may send, at step 708-5, each piece or set of data and/or the fused data, if available, to the report module 142-5. The data fusion module 140-5 may return, at step 710-5, to step 700-5.

FIG. 44 illustrates an example operation of the report module 142-5 of the wearable device 136-5. The process may begin with the report module 142-5 polling, at step 800-5, for data from the data fusion module 140-5. The report module 142-5 may display, at step 802-5, the received data via the display 146-5. The report module 142-5 may also send data in real-time or periodically. Data may be sent to an email address, a database, a module, or any other destination. The data may be sent to and/or displayed at multiple locations. The user may use this data to assist with diseases or illnesses. For example, a user is diabetic. Glucose readings are taken from the user by both the device 108-5 and a sensor 148-5, which detects glucose in sweat. The results are fused such that the number of false spikes and drops in glucose due to an issue with the device 108-5 or sensor 148-5 is reduced or eliminated entirely. When the user views a display of their glucose level over time, they may be able to view each recording and the fused data to see where the false spikes and drops would have been if there was no data fusion. For another example, a user has hypertension, and their blood pressure is monitored using both a device 108-5 and a smartphone app. The wearable device measures blood pressure continuously throughout the day while the app prompts the user to measure their blood pressure at regular intervals. The results from both sources are fused using a machine learning algorithm, which considers factors such as activity level, sleep quality, and diet. The fused data provides a more accurate and comprehensive picture of the user's blood pressure over time, allowing for better management of hypertension and the prevention of complications. For another example, a user has asthma, and their lung function is monitored using a spirometer, a pulse oximeter, and a device 108-5 measuring blood oxygen level. The spirometer measures lung function by having the user blow into a mouthpiece, while the pulse oximeter measures the oxygen saturation in the user's blood. The results from all three devices are fused using a data fusion algorithm, which considers factors such as the user's age, gender, and smoking history. The fused data provides a more accurate and comprehensive picture of the user's lung function over time, allowing for better asthma management and the prevention of exacerbations. The report module 142-5 may return, at step 804-5, to step 800-5.

In one example implementation of Embodiment 5, a method for measuring one or more analytes using a real-time, non-invasive radio frequency (“RF”) analyte detection device cam include the following: obtaining a first set of data from the real-time, non-invasive RF analyte detection device; obtaining a second set of data from a wearable device comprising a sensor; executing a connection module to retrieve the first set of data from the real-time, non-invasive RF analyte detection device and the second set of data from the sensor of the wearable device; executing a fusion module to combine the first set of data obtained from the real-time, non-invasive RF analyte detection device and the second set of data from the sensor; and reporting the fused data results.

### Embodiment 6

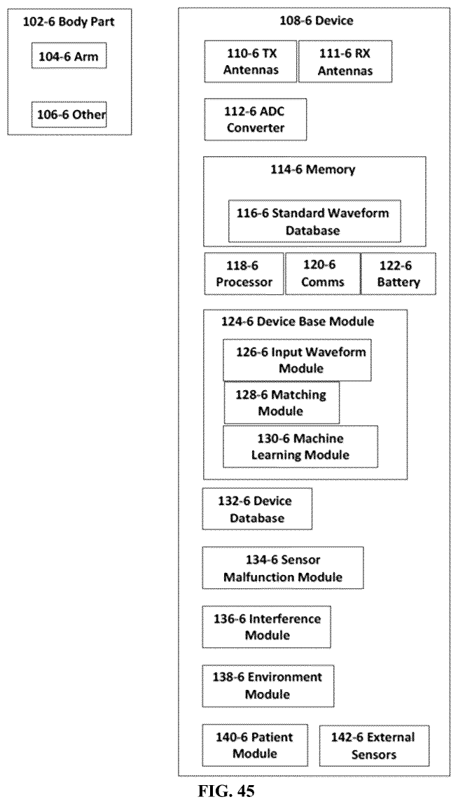

FIG. 45 is a schematic illustration of a system for radio frequency health monitoring. This system is configured to be attached or in proximity to a body part 102-6. The body part 102-6 may be an arm 104-6. The body part 102-6 may be another body part 106-6 besides an arm, such as a leg, finger, chest, head, or any other body part from which useful medical parameters can be taken.

The system may comprise a device 108-6, which may be a wearable and portable device such as, but not limited to, a cell phone, a smartwatch, a tracker, a wearable monitor, a wristband, and a personal blood monitoring device.

The system may further comprise one or more transmit (“TX”) antennas 110-6. The one or more TX antennas 110-6 may be configured to transmit RF in the RF Activated Range from 500 MHZ to 300 GHZ. A pre-defined frequency may correspond to a range suitable for the human body in one embodiment. For example, the one or more TX antennas 110-6 would transmit radio frequency signals at a range of 120-126 GHz. The system may further comprise one or more receive (“RX”) antennas 111-6. The one or more RX antennas 111-6 may be configured to receive the RF signals in response to the RF signal transmitted from the one or more TX antennas 110-6.

The system may further comprise an ADC converter 112-6, which may be configured to convert the received RF signals from an analog signal into a digital processor readable format.

The system may further comprise memory 114-6, which may be configured to store the transmitted RF signals by the one or more TX antennas 110-6 and store the received portion of the response or responded RF signals from the one or more RX antennas 111-6. Further, the memory 114-6 may also store the converted digital processor readable format by the ADC converter 112-6. The memory 114-6 may include suitable logic, circuitry, and/or interfaces that may be configured to store a machine code and/or a computer program with at least one code section executable by the processor 118-6. Examples of implementation of the memory 114-6 may include, but are not limited to, Random Access Memory (RAM), Read Only Memory (ROM), Hard Disk Drive (HDD), and/or a Secure Digital (SD) card.

The system may further comprise a standard waveform database 116-6, which may contain standard waveforms for known patterns. These may be raw or converted device readings from patients or persons with known conditions. For example, the standard waveform database 116-6 may include raw or converted device readings from a patient known to have diabetes or an average of multiple patients. This data can be compared to readings from a person with an unknown condition in order to determine if the waveforms taken from that person match any of the known standard waveforms. The device 108-6 may be used on animals as well as people.

The system may further comprise a processor 118-6, which may facilitate the operation of the device 108-6 according to the instructions stored in the memory 114-6. The processor 118-6 may include suitable logic, circuitry, interfaces, and/or code that may be configured to execute a set of instructions stored in the memory 114-6.

The system may further comprise a comms 120-6, which may be a communication element such as a Wi-Fi or RFC transmitter and receiver or physical connection that can send and receive data to other devices of the system and devices external to the system. The comms 120-6 may be configured to comply with regulatory acts such as HIPPA. For example, the comms 120-6 may include features such as encryption, access control, audit trails, secure transmission, credential verification, or other features which may protect the integrity and privacy of transmitted or received data.

The system may further comprise a battery 122-6, which may power hardware modules of the device 108-6. The device 108-6 may be configured with a charging port to recharge the battery 122-6. Charging of the battery 122-6 may be wired or wireless.

The system may further comprise a device base module 124-6, which may be configured to store instructions for executing the computer program from the converted digital processor readable format of the ADC converter 112-6. The device base module 124-6 may be configured to facilitate the operation of the processor 118-6, the memory 114-6, the one or more TX antennas 110-6, the one or more RX antennas 111-6, and the comms 120-6. Further, the device base module 124-6 may be configured to create polling of the RF Activated Range from 500 MHZ to 300 GHZ. It can be noted that the device base module 124-6 may be configured to filter the RF Activated Range from 500 MHZ to 300 GHZ received from one or more RX antennas 111-6.

The system may further comprise an input waveform module 126-6, which may extract a radio frequency waveform from memory. This may be the raw or converted recording of data from the one or more RX antennas 111-6 from a patient wearing the device. If the entire radio frequency is too long for effective matching, the input waveform module 126-6 may select a time interval within the data set. This input waveform may then be sent to the matching module 128-6.

The system may further comprise a matching module 128-6, which may match the input waveform and each of the standard waveforms in the standard waveform database 116-6 by performing a convolution and/or cross-correlation of the input waveform and the standard waveform. These convolutions and/or cross-correlations are then sent to the machine learning module.

The system may further comprise a machine learning module 130-6 which has been trained to identify health parameters based on the convolution and/or cross-correlations of the input and standard waveforms. The machine learning module 130-6 receives the convolutions and cross-correlations from the matching module 128-6 and outputs any health parameters identified.

The system may further comprise a device database 132-6, which may store the health parameters output by the machine learning module 130-6.

The system may further comprise a sensor malfunction module 134-6, which may ensure that continuous RF readings are maintained by detecting and responding to any issues with the sensor or device 108-6. The sensor malfunction module may provide real-time alerts and guidance to help healthcare providers, and patients take prompt action to address any issues and minimize any disruptions to the monitoring process.

The system may further comprise an interference module 136-6, which may continuously monitor the RF sensor and the surrounding environment for any sources of electromagnetic interference that could disrupt or distort the sensor readings. The interference module 136-6 may then alter the signal strength or frequency to reduce or eliminate the interference.

The system may further comprise an environment module 138-6, which may continuously monitor the RF sensor and the surrounding environment for any factors that could impact the accuracy and reliability of the sensor readings, such as temperature or humidity.

The system may further comprise a patient module 140-6, which may continuously monitor the patient and their individual factors that could impact the accuracy and reliability of the RF sensor readings, such as skin dryness, oiliness, or thickness, the location of the device on the patient, movement by the patient, the patient's age, or weight, etc.

The system may further comprise one or more sensors 142-6 which allow the device to measure parameters such as electromagnetic waves, temperature, humidity, patient skin conditions, patient movement, or any other parameter which may contribute to disruptions, errors, noise, or lack of accuracy of the device 108-6.

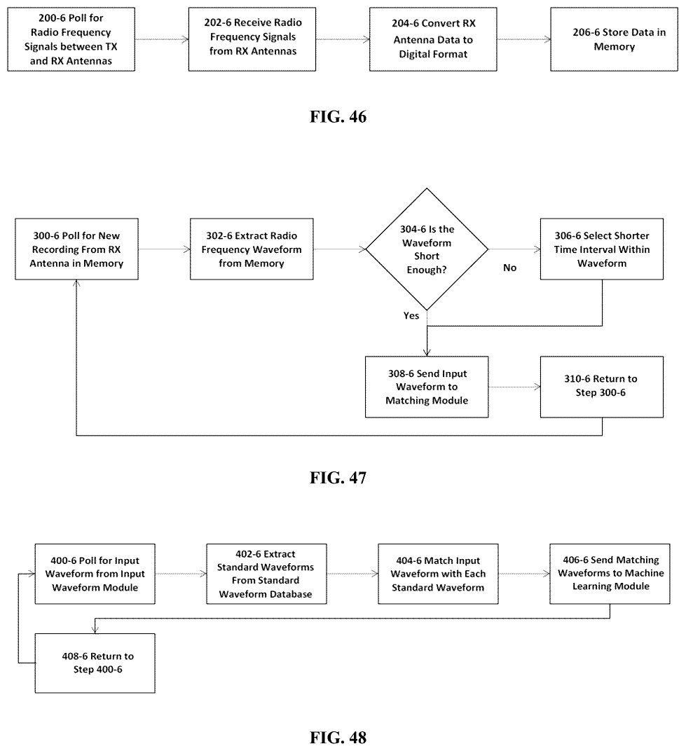

FIG. 46 illustrates an example operation of the device base module 124-6. The process may begin with the device base module 124-6 polling the Active Range RF signals between the one or more TX antennas 110-6 and the one or more RX antennas 111-6 at step 200-6. The device base module 124-6 may be configured to read and process instructions stored in the memory 114-6 using the processor 118-6. The one or more TX antennas 110-6 may be configured to transmit RF in the RF Activated Range from 500 MHZ to 300 GHZ. For example, the one or more TX antennas 110-6 may use RF signals at a range of 500 MHZ to 300 GHZ. The device base module 124-6 may receive the RF frequency signals from the one or more RX antennas 111-6 at step 202-6. For example, one or more RX antennas 111-6 receives a response or responded RF of frequency range 300-330 GHz from the patient's blood. The device base module 124-6 may be configured to convert the received RF signals into a digital format using the ADC 112-6 at step 204-6. For example, the received RF of 300-330 GHz frequency range is converted into a 10-bit data signal. The device base module 124-6 may be configured to store converted digital format into the memory 114-6 at step 206-6.

FIG. 47 illustrates an example operation of the input waveform module 126-6. The process may begin with the input waveform module 126-6 polling, at step 300-6, for new recorded data from the one or more RX antennas 111-6 stored in memory 114-6. The input waveform module 126-6 may extract, at step 302-6, the recorded radio frequency waveform from memory. If there is more than one waveform recorded, the input waveform module 126-6 may select each waveform separately and loop through the following steps. The input waveform module 126-6 may determine, at step 304-6, if the waveform is small enough to be an input waveform for the matching module 128-6. This will depend on the computational requirements and/or restrictions of the matching module 128-6. If the waveform is short enough, the input waveform module 126-6 may skip to step 308-6. If the waveform is too long, the input waveform module 126-6 may select, at step 306-6, a shorter time interval within the entire recorded waveform. For example, if the waveform is 5 minutes long, then only a 30-second interval may be selected. The interval may be selected at random or by a selection process. The input waveform module 126-6 may send, at step 308-6, the input waveform to the matching module 128-6. The input waveform module 126-6 may return, at step 310-6, to step 300-6.

FIG. 48 illustrates an example operation of the matching module 128-6. The process may begin with the matching module 128-6 polling, at step 400-6, for an input waveform from the input waveform module 126-6. The matching module 128-6 may extract, at step 402-6, each standard waveform from the standard waveform database 116-6. The matching module 128-6 may match, at step 404-6, the input waveform with each standard waveform. Matching may be determining which standard waveforms the input waveform is similar to. Matching may involve convolution and/or cross-correlation of the waveforms or any other matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function whose values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-6. The matching module 128-6 may send, at step 406-6, the matching waveforms to the machine learning module 130-6. Matching waveforms may refer to the standard waveforms similar to the input waveform, the waveforms generated via convolution and/or cross-correlation, or both. The matching module 128-6 may return, at step 408-6, to step 400-6.

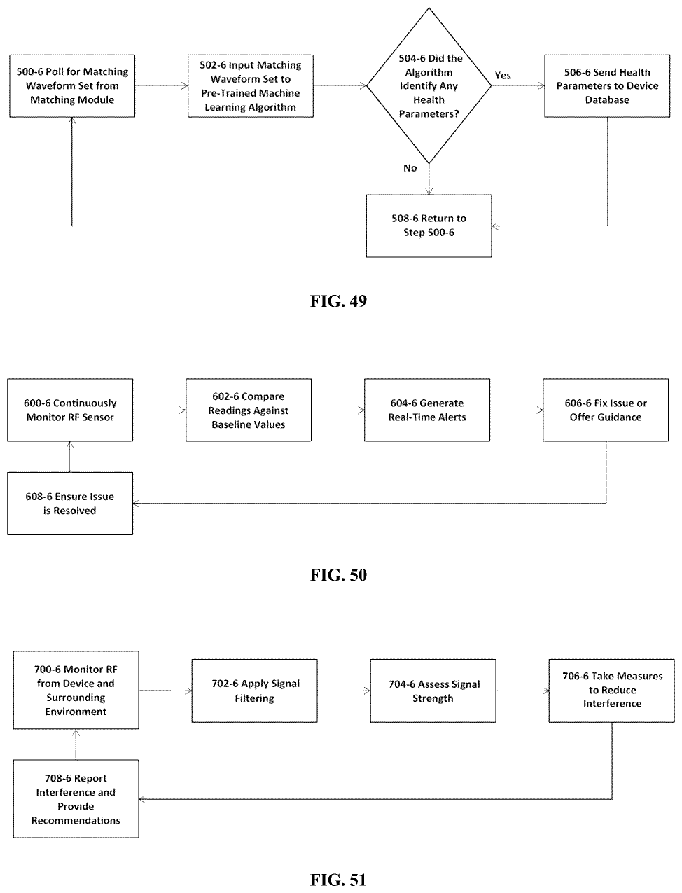

FIG. 49 illustrates an example operation of the machine learning module 130-6. The process may begin with the machine learning module 130-6 polling, at step 500-6, for a set of matching waveforms from the matching module 128-6. Matching waveforms may be a set of standard waveforms similar to the input waveform or statistical combinations of the input waveform and standard waveforms, such as convolutions or cross-correlations. The machine learning module 130-6 may input, at step 502-6, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During the training process, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-6 may determine, at step 504-6, if the algorithm identified any health parameters. Identification may require a certain interval of confidence. For example, if the machine learning algorithm determines that it is more than 70% likely that a health parameter is correct, that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130-6 may skip to step 508-6. If any health parameters were identified, the machine learning module 130-6 may send, at step 506-6, the health parameters to the device database 132-6. The machine learning module 130-6 may return, at step 508-6, to step 500-6.

FIG. 50 illustrates an example operation of the sensor malfunction module 134-6. The sensor malfunction module 134-6 may continuously assess at step 600-6 the performance and readings of the device 108-6 to ensure it maintains optimal functioning and accurately captures the patient's health parameters. For example, the sensor malfunction module 134-6 may keep track of signal strength and quality, checking for sudden drops or spikes that could indicate a malfunction. The sensor malfunction module 134-6 may compare, at step 602-6, current readings against established baseline values or historical data to spot any irregularities or discrepancies that might signal a malfunction. For example, device 108-6 typically detects a glucose level of 60-100 mg/dL but suddenly records a rate of 200 mg/dL. This could indicate a potential issue with the sensor. The sensor malfunction module 134-6 could use various algorithms and machine learning techniques to analyze the sensor readings and identify any patterns or anomalies that suggest a malfunction or deterioration of the sensor. The sensor malfunction module 134-6 may generate, at step 604-6, alerts and notifications for healthcare providers and patients when a potential issue is detected, offering information about the problem and its potential consequences for the monitoring process. For example, if the sensor malfunction module 134-6 detects a weak or inconsistent signal from the RF sensor, it could send an alert to the patient's smartwatch or smartphone and their healthcare provider's monitoring system, informing them of the issue and its potential impact on data reliability. The sensor malfunction module 134-6 may solve, at step 606-6, the issue automatically if possible or offer recommendations on resolving the detected issue, which may include device troubleshooting, adjusting the device's position on the patient's body, or seeking professional assistance. If the sensor malfunction module 134-6 determines that the issue is due to poor contact between the TX antennas 110-6 and the patient's skin, it could move the antennas 110-6, boost the signal, or suggest that the patient reposition the device, clean the sensor, or apply a conductive gel to improve signal quality. For example, the sensor malfunction module 134-6 could automatically switch to a backup sensor if one is available, or it could adjust the sensitivity or gain of the sensor to compensate for any loss in performance. The sensor malfunction module 134-6 may continue, at step 608-6, to oversee the situation after providing guidance to ensure that the issue has been resolved and the RF sensor is functioning correctly. For example, if the patient follows the provided guidance to reposition the device 108-6, the sensor malfunction module 134-6 will continue to monitor the sensor's performance and signal quality, confirming that the issue has been resolved and that the RF sensor is capturing accurate and reliable data. The sensor malfunction module 134-6 may then return to step 600-6.

FIG. 51 illustrates an example operation of the interference module 136-6. The interference module 136-6 may monitor, at step 700-6, the device 108-6 performance and signal quality, as well as the presence of any external signals or potential sources of interference in the environment using one or more of the sensors 142-6. The interference module 136-6 may apply, at step 702-6, digital signal processing techniques to filter out unwanted noise or interference from the sensor readings, such as bandpass filters or adaptive filtering algorithms. For example, if a nearby Wi-Fi router is causing interference, the interference module 136-6 may apply a bandpass filter to isolate the desired frequency range and remove the Wi-Fi signal from the sensor readings. The interference module 136-6 may assess, at step 704-6, the strength of the signal between the TX antennas 110-6 and RX antennas 111-6 and compare it to the strength of other signals in the environment, such as Wi-Fi or Bluetooth signals, to determine if interference is affecting the sensor readings. For example, if the device 108-6 signal strength has dropped below a certain threshold due to interference, the interference module 136-6 could alert the user or healthcare provider to take action. The interference module 136-6 may implement, at step 706-6, measures to reduce interference, such as boosting the output from the TX antennas 110-6 or using frequency hopping techniques to switch the TX antennas 110-6 to different frequency channels in real-time to avoid interference from other signals in the environment. For example, if the interference module 136-6 detects a strong interfering signal at the current frequency, it could switch the device 108-6 to an alternate frequency channel with less interference. The interference module 136-6 may report, at step 708-6, real-time feedback to the doctor, patient, or healthcare provider on the quality and reliability of the device 108-6 readings, such as a signal quality indicator or a signal-to-noise ratio measurement. For example, the interference module 136-6 could display a signal quality indicator on the user's smartwatch, smartphone, or healthcare provider's monitoring system, allowing them to quickly identify any potential interference issues. The interference module 136-6 may recommend measures to reduce interference, such as repositioning the device, adjusting the environment, or disabling interfering devices. For example, if the interference module 136-6 determines that a nearby Bluetooth speaker is causing interference, it could recommend turning off the speaker or moving it further away from the device 108-6. The interference module may then return to step 700-6.

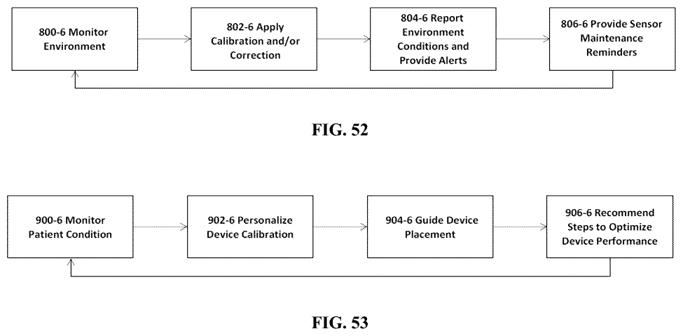

FIG. 52 illustrates an example operation of the environment module 138-6. The environment module 138-6 may monitor, at step 800-6, the surrounding environment's parameters, such as temperature and humidity levels using the sensors 142-6, and assess their impact on device 108-6 performance. The environment module 138-6 may apply, at step 802-6, calibration algorithms to adjust the sensor readings based on environmental conditions, compensating for any changes in the signal due to temperature, humidity, dust, or other particles in the air, etc. For example, if the temperature increases, the environment module 138-6 could adjust the sensor readings to account for the change in signal strength or transmission characteristics caused by the higher temperature, which can cause minor changes in how air interacts with radio frequency waves. The environment module 138-6 may report, at step 804-6, the environmental conditions to a patient or healthcare provider. If the temperature or humidity levels exceed certain thresholds, the environment module 138-6 could alert the patient or healthcare provider and recommend measures to stabilize the environment. For example, if the humidity level rises significantly, the environment module 138-6 could send an alert to the patient's smartphone, suggesting they move to a less humid environment or use a dehumidifier to stabilize the conditions. The environment module 138-6 may provide, at step 806-6, reminders for sensor 142-6 maintenance tasks, such as cleaning or replacing the sensor, to ensure that it operates at peak performance. For example, the environment module 138-6 could send a notification to the user's smartphone or healthcare provider's monitoring system, reminding them to clean a sensor 142-6 or replace it after a specific period of use. The environment module 138-6 may then return to step 800-6.

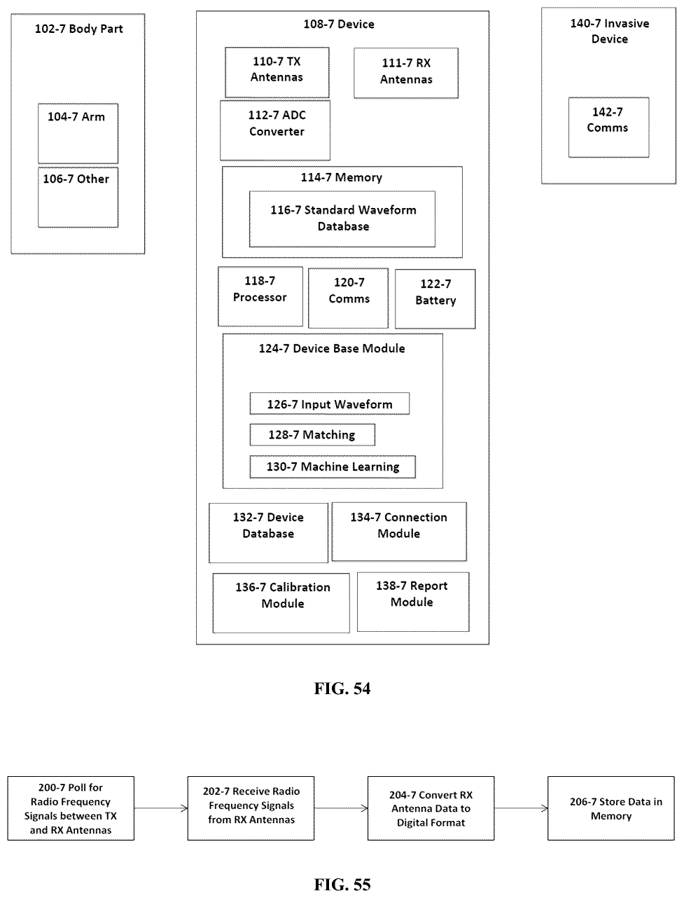

FIG. 53 illustrates an example operation of the patient module 140-6. The patient module 140-6 may monitor, at step 900-6, the patient's condition, such as skin dryness or oiliness, moving or stationary, device location, etc., using the sensors 142-6 and determine any factors that could impact sensor readings. For example, the patient module 140-6 could detect that the patient's skin is particularly dry, potentially affecting the sensor's contact and signal quality. The patient module 140-6 may use, at step 902-6, data from the patient's individual factors, such as age or weight, to personalize the calibration of the sensor and optimize its performance for the specific patient. For example, the patient module 140-6 may adjust the calibration algorithm to account for a patient's higher body mass index (BMI) and, therefore, more tissue for the RF waves to pass through, ensuring more accurate and reliable readings. The patient module 140-6 may provide, at step 904-6, guidance to the patient or healthcare provider on the optimal placement of the device 108-6 based on the patient's individual factors, such as skin condition or body type. For example, the patient module 140-6 could recommend placing the sensor on a specific location, such as the upper arm, based on the patient's body type and skin condition for better performance. The patient module 140-6 may recommend, at step 906-6, measures to the patient or healthcare provider to optimize sensor performance based on the skin condition analysis. For example, the patient module 140-6 may suggest the patient apply lotion or clean the skin to improve sensor contact and signal quality. The patient module 140-6 may return to step 900-6.

The system may operate for various durations sufficient to detect errors in the measurements of any analytes. Examples of such durations include, but are not limited to: A duration of approximately 50 to 500 milliseconds, which may be suitable for performing rapid checks on the functionality of the device and the integrity of the analyte measurement system; A duration of approximately 1 to 5 seconds, which may be adequate for detecting transient errors or fluctuations in analyte measurements caused by short-term disturbances, such as motion artifacts or brief changes in physiological conditions; A duration of approximately 5 to 10 minutes, which may be appropriate for monitoring the effects of treatments or interventions on analyte levels, allowing for the identification of potential errors related to these factors; A duration of approximately 30 minutes to 1 hour, which may be suitable for assessing the impact of environmental factors or patient positions on analyte measurements, providing ample time for detecting errors associated with these variables; A duration of approximately 1 to 3 hours, which may be necessary for continuous monitoring of analyte levels in patients with acute conditions or those undergoing diagnostic procedures, enabling the system to identify errors or inconsistencies in the measurements over an extended period; A duration of approximately 6 to 12 hours or more, which may be appropriate for continuous monitoring of analyte levels in patients with chronic conditions or those requiring long-term care, allowing the system to detect errors in the measurements over an extended timeframe.

In one example implementation of Embodiment 6, a method for measuring one or more analytes using a real-time, non-invasive radio frequency (“RF”) analyte detection device can include the following: providing the real-time, non-invasive RF analyte detection device; providing an uninterrupted stream of data recorded from a patient by the real-time, non-invasive RF analyte detection device; providing a module to prevent errors in the uninterrupted stream of data caused by a malfunction of the real-time, non-invasive RF analyte detection device; providing a module to prevent errors in the uninterrupted stream of data caused by electromagnetic interference; providing a module to prevent errors in the uninterrupted stream of data caused by environmental factors; and providing a module to prevent errors in the uninterrupted stream of data caused by the patient; wherein the uninterrupted stream of data lasts for a duration to ensure errors in the data stream are capable of being corrected.

### Embodiment 7

FIG. 54 is a schematic illustration of a system for radio frequency health monitoring. This system is configured to be attached to or in proximity to a body part 102-7. The body part 102-7 may be an arm 104-7. The body part 102-7 may be another body part besides an arm, such as a leg, finger, chest, head, or any other body part from which useful medical parameters can be taken.

The system may comprise a device 108-7, which may be a wearable and portable device such as, but not limited to, a cell phone, a smartwatch, a tracker, a wearable monitor, a wristband, and a personal blood monitoring device.

The system may further comprise one or more transmit (“TX”) antennas 110-7. The one or more TX antennas may be configured to transmit RF in the RF Activated Range from 500 MHZ to 300 GHZ. A pre-defined frequency may correspond to a range suitable for the human body in one embodiment. For example, the one or more TX antennas would transmit radio frequency signals at a range of 120-126 GHz.

The system may further comprise one or more receive (“RX”) antennas 111-7. The one or more RX antennas may be configured to receive the RF signals in response to the RF signal transmitted from the one or more TX antennas.

The system may further comprise an ADC converter 112-7, which may be configured to convert the received RF signals from an analog signal into a digital processor readable format. The system may further comprise memory 114-7, which may be configured to store the transmitted RF signals by the one or more TX antennas 110-7 and store the received portion of the response or responded RF signals from the one or more RX antennas 111-7. Further, the memory 114-7 may also store the converted digital processor readable format by the ADC converter 112-7. The memory 114-7 may include suitable logic, circuitry, and/or interfaces that may be configured to store a machine code and/or a computer program with at least one code section executable by the processor 118-7. Examples of implementation of the memory 114-7 may include, but are not limited to, Random Access Memory (RAM), Read Only Memory (ROM), Hard Disk Drive (HDD), and/or a Secure Digital (SD) card. The system may further comprise a standard waveform database 116-7, which may contain standard waveforms for known patterns. These may be raw or converted device readings from patients or persons with known conditions. For example, the standard waveform database 116-7 may include raw or converted device readings from the right arm of a patient known to have diabetes or an average of multiple patients. This data can be compared to readings from a person with an unknown condition in order to determine if the waveforms taken from that person match any of the known standard waveforms. The device 108-7 may be used on animals as well as people.

The system may further comprise a processor 118-7, which may facilitate the operation of the device 108-7 according to the instructions stored in the memory 114-7. The processor 118-7 may include suitable logic, circuitry, interfaces, and/or code that may be configured to execute a set of instructions stored in the memory 114-7.

The system may further comprise a comms 120-7, which may be a communication element such as a Wi-Fi or RFC transmitter and receiver or physical connection that can send and receive data to other devices of the system and devices external to the system. The comms 120-7 may be configured to comply with regulatory acts such as HIPPA. For example, the comms 120-7 may include features such as encryption, access control, audit trails, secure transmission, credential verification, or other features which may protect the integrity and privacy of transmitted or received data.

The system may further comprise a battery 122-7, which may power hardware modules of the device 108-7. The device 108-7 may be configured with a charging port to recharge the battery 122-7. Charging of the battery 122-7 may be wired or wireless.

The system may further comprise a device base module 124-7, which may be configured to store instructions for executing the computer program from the converted digital processor readable format of the ADC converter 112-7. The device base module 124-7 may be configured to facilitate the operation of the processor 118-7, the memory 114-7, the one or more TX antennas 110-7, the one or more RX antennas 111-7, and the comms 120-7. Further, the device base module 124-7 may be configured to create polling of the RF Activated Range from 500 MHZ to 300 GHZ. It can be noted that the device base module 124-7 may be configured to filter the RF Activated Range from 500 MHZ to 300 GHZ received from one or more RX antennas 111-7.

The system may further comprise an input waveform module 126-7, which may extract a radio frequency waveform from memory. This may be the raw or converted recording of data from the one or more RX antennas 111-7 from a patient wearing the device. If the entire radio frequency is too long for effective matching, the input waveform module 126-7 may select a time interval within the data set. This input waveform may then be sent to the matching module 128-7.

The system may further comprise a matching module 128-7, which may match the input waveform and each of the standard waveforms in the standard waveform database 116-7 by performing a convolution and/or cross-correlation of the input waveform and the standard waveform. These convolutions and/or cross-correlations are then sent to the machine learning module.

The system may further comprise a machine learning module 130-7 which has been trained to identify health parameters based on the convolution and/or cross-correlations of the input and standard waveforms. The machine learning module 130-7 receives the convolutions and cross-correlations from the matching module 128-7 and outputs any health parameters identified.

The system may further comprise a device database 132-7, which may store the health parameters output by the machine learning module 130-7.

The system may further comprise a connection module 134-7, which may connect to the invasive device 140-7 and collect data which then may be stored in the device database 132-7.

The system may further comprise a calibration module 136-7, which may calibrate the device 108-7 based on the collected data real-time analyte data from the invasive device 140-7 stored in the device database 132-7. Data from the device 108-7 is compared to the invasive device 140-7, and the output of the device 108-7 may be adjusted to match the readings from the invasive device.

The calibration module 136-7 may be run continuously for initial calibration and then periodically to check that the device 108-7 is still calibrated.

The system may further comprise a report module 138-7, which may display a report of the calibration process's success or failure and the degree thereof. This report may be sent to an email address, displayed on a screen, sent to another module, or otherwise reported.

The system may further comprise an invasive device 140-7, which may be any device or sensor inserted into a patient's bloodstream or at least under the skin to measure analyte levels.

The system may further comprise the comms 142-7, which may be a communication element such as a Wi-Fi or RFC transmitter and receiver or physical connection that can send and receive data to other devices of the system and devices external to the system. The comms 142-7 may be configured to comply with regulatory acts such as HIPPA. For example, the comms 142-7 may include features such as encryption, access control, audit trails, secure transmission, credential verification, or other features which may protect the integrity and privacy of transmitted or received data.

FIG. 55 illustrates an example operation of the device base module 124-7. The process may begin with the device base module 124-7 polling the Active Range RF signals between the one or more TX antennas 110-7 and the one or more RX antennas 111-7 at step 200-7. The device base module 124-7 may be configured to read and process instructions stored in the memory 114-7 using the processor 118-7. The one or more TX antennas 110-7 may be configured to transmit RF in the RF Activated Range from 500 MHZ to 300 GHZ. For example, the one or more TX antennas 110-7 may use RF signals at a range of 500 MHZ to 300 GHZ. The device base module 124-7 may receive the RF frequency signals from the one or more RX antennas 111-7 at step 202. For example, one or more RX antennas 111-7 receives a response or responded RF of frequency range 300-330 GHz from the patient's blood. The device base module 124-7 may be configured to convert the received RF signals into a digital format using the ADC 112 at step 204-7. For example, the received RF of 300-330 GHz frequency range is converted into a 10-bit data signal. The device base module 124-7 may be configured to store converted digital format into the memory 114-7 at step 206-7.

FIG. 56 illustrates an example operation of the input waveform module 126-7. The process may begin with the input waveform module 126-7 polling, at step 300-7, for new recorded data from the one or more RX antennas 111-7 stored in memory 114-7. The input waveform module 126-7 may extract, at step 302-7, the recorded radio frequency waveform from memory. If there is more than one waveform recorded, the input waveform module 126-7 may select each waveform separately and loop through the following steps. The input waveform module 126-7 may determine, at step 304-7, if the waveform is small enough to be an input waveform for the matching module 128-7. This will depend on the computational requirements and/or restrictions of the matching module 128-7. If the waveform is short enough, the input waveform module 126-7 may skip to step 308-7. If the waveform is too long, the input waveform module 126-7 may select, at step 306-7, a shorter time interval within the entire recorded waveform. For example, if the waveform is 5 minutes long, then only a 30-second interval may be selected. The interval may be selected at random or by a selection process. The input waveform module 126-7 may send, at step 308-7, the input waveform to the matching module 128-7. The input waveform module 126-7 may return, at step 310-7, to step 300-7.

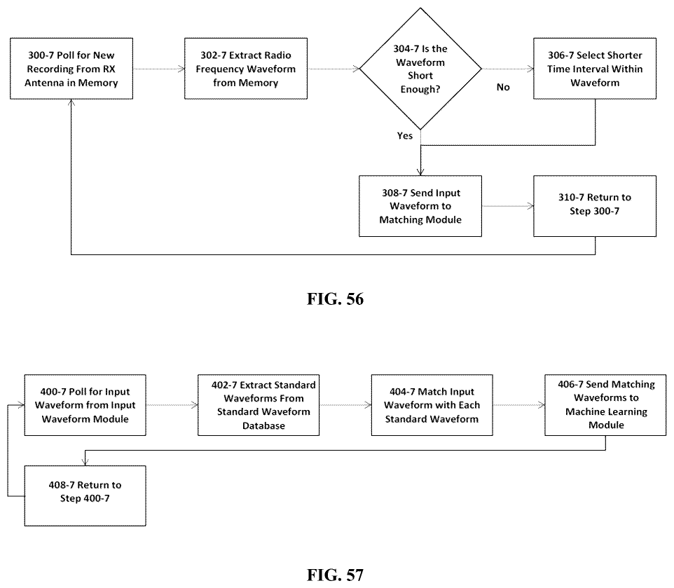

FIG. 57 illustrates an example operation of the matching module 128-7. The process may begin with the matching module 128-7 polling, at step 400-7, for an input waveform from the input waveform module 126-7. The matching module 128-7 may extract, at step 402-7, each standard waveform from the standard waveform database 116-7. The matching module 128-7 may match, at step 404-7, the input waveform with each standard waveform. Matching may be determining which standard waveforms the input waveform is similar to. Matching may involve convolution and/or cross-correlation of the waveforms or any other matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function whose values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-7. The matching module 128-7 may send, at step 406-7, the matching waveforms to the machine learning module 130-7. Matching waveforms may refer to the standard waveforms similar to the input waveform, the waveforms generated via convolution and/or cross-correlation, or both. The matching module 128-7 may return, at step 408-7, to step 400-7.

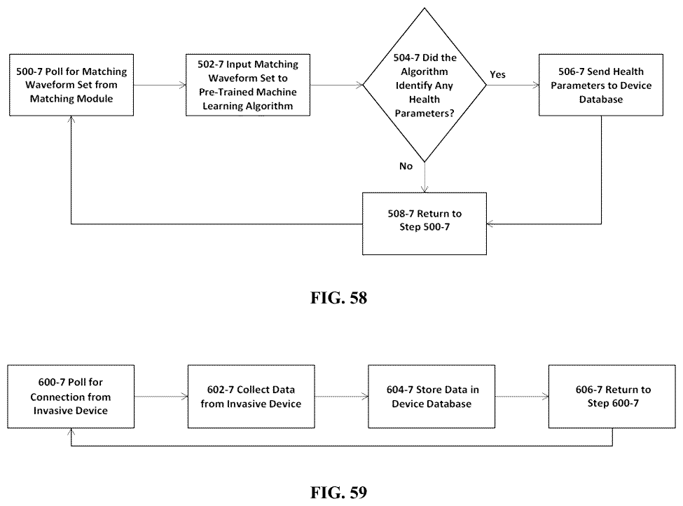

FIG. 58 illustrates an example operation of the machine learning module 130-7. The process may begin with the machine learning module 130-7 polling, at step 500-7, for a set of matching waveforms from the matching module 128-7. Matching waveforms may be a set of standard waveforms similar to the input waveform, or statistical combinations of the input waveform and standard waveforms, such as convolutions or cross-correlations. The machine learning module 130-7 may input, at step 502-7, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During the training process, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-7 may determine, at step 504-7, if the algorithm identified any health parameters. Identification may require a certain interval of confidence. For example, if the machine learning algorithm determines that it is more than 70% likely that a health parameter is correct, that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130-7 may skip to step 508-7. If any health parameters were identified, the machine learning module 130-7 may send, at step 506-7, the health parameters to the device database 132-7. The machine learning module 130-7 may return, at step 508-7, to step 500-7.

FIG. 59 illustrates an example operation of the connection module 134-7. The connection module 134-7 may poll, at step 600-7, for a connection from the invasive device 140-7. If no connection is detected, the connection module 134-7 may search for an invasive device 140-7 attempting to connect or skip to step 604-7. The connection module 134-7 may collect, at step 602-7, data from the invasive device 140-7. This may be real-time data or periodically updated data. An example of an invasive device 140-7 may be a glucose monitoring system for a patient with diabetes. The connection module 134-7 may store, at step 604-7, the data from the invasive device 140-7 in the device database 132-7. The connection module 134-7 may add contextual data such as the ID of the invasive device 140-7, type of invasive device 140-7, analyte measured by the invasive device 140-7, time data was retrieved, etc., to the received data before storage in the device database 132-7. The connection module 134-7 may return, at step 606-7, to step 600-7.

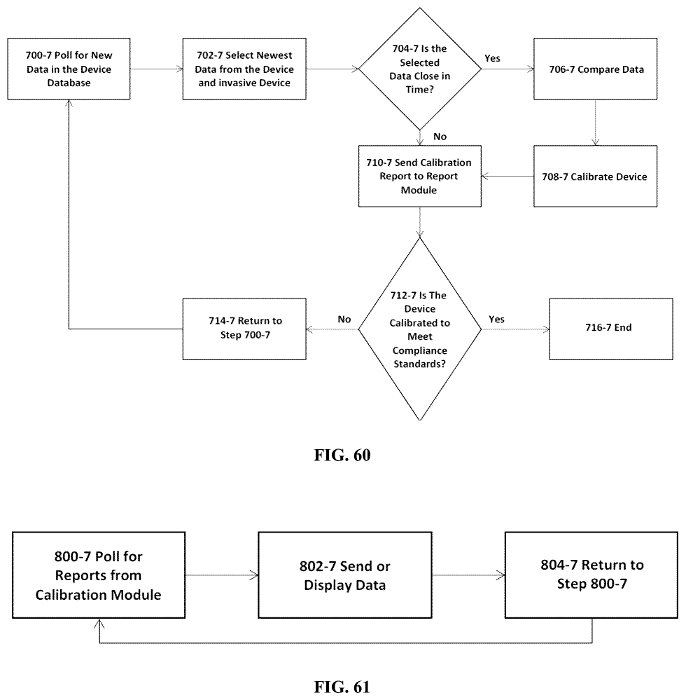

FIG. 60 illustrates an example operation of the calibration module 136-7. The calibration module 136-7 may poll, at step 700-7, for new data in the device database 132-7. This new data may be from the device 108-7, and/or from the invasive device 140-7. The calibration module 136-7 may select, at step 702-7, the newest data from the device 108-7, and the newest data from the invasive device 140-7. This may be a single data point or a set of data. The calibration module 136-7 may determine, at step 704-7, if the selected data is close in time. Close in time may mean each piece or set of data is timestamped within a few seconds of each other. For example, the newest data from the device 108-7 may be real-time, but the newest reading from the invasive device 140-7 may have been 10 minutes ago. This data would not be close in time if the threshold is a few seconds. The threshold may be varied based on the health parameter being measured. For example, if the invasive device 140-7 is recording slowly changing analytes such as platelet count, then recordings from 10 minutes ago may be relevant because of the gradual nature of sleep cycles, but if the invasive device 140-7 is recording blood glucose or electrolyte levels, then data from minutes ago may be highly inaccurate. The time threshold for what data is considered close in time may be set by a device user, manufacturer of the device, and/or another module. If the selected data is not close in time, the calibration module may skip to step 710-7 and report a failure to calibrate. The calibration module 136-7 may compare, at step 706-7, the data obtained from the device 108-7 with the data collected from the invasive device 140-7. This comparison helps assess the device's accuracy and identify any discrepancies between the two sets of measurements. The calibration module 136-7 may utilize statistical techniques for comparing time-synchronized data obtained from the device 108-7 and the invasive device 140-7. These techniques may include error analysis methods, such as calculating the Mean Absolute Error (MAE) or Root Mean Square Error (RMSE) between corresponding data points, as well as regression analysis methods, such as fitting a linear regression model to the data and evaluating the coefficient of determination (R2) to assess the degree of agreement between the two data sets. By employing these comparison methods, the system can identify discrepancies or trends in the data, enabling necessary adjustments to the algorithms to enhance the accuracy and reliability of the non-invasive RF sensor system. The calibration module 136-7 may wait until enough time-synchronized data points are obtained from the device 108-7 and invasive device 140-7 to perform a comprehensive comparison. These datapoints may be temporarily marked in the device database 132-7 or stored in a separate database. The calibration module 136-7 may adjust, at step 708-7, the algorithms used by the device 108-7 to better match the data from the invasive device 140-7 based on the comparison results. This may include fine-tuning the parameters of the algorithms, updating the standard waveform database 116-7, retraining the algorithm used by the machine learning module 130-7, or modifying other aspects of the system to improve measurement accuracy. The calibration module 136-7 may employ adaptive learning techniques to continuously refine the calibration process as more data is collected. This feature allows the system to improve its performance over time, adapting to the patient's unique physiology and changes in their health condition. The calibration module 136-7 may generate, at step 710-7, generate reports on the success or failure of the calibration process, as well as the degree of improvement achieved. These reports may be sent to the report module 138-7. These reports can be sent to an email address, displayed on a screen, forwarded to another module, or otherwise communicated to the user or healthcare provider. The calibration module 136-7 may determine, at step 712-7, if the device 108-7 is calibrated to meet compliance standards. For example, compliance standards may require the device 108-7 to give results within 0.5% of the results given by the invasive device 140-7. If the results from the device 108-7 are outside of this range within a set time period, such as over 24 hours, then the device 108-7 may not be compliant and may require further calibration or recalibration. Compliance standards may refer to legal standards, industry standards, standards set by the patient or their healthcare professionals, standards set by the manufacturer, etc. If multiple standards are used, the strictest standard may be used. If the device 108-7 does not meet compliance standards, the calibration module 136-7 may return to step 700-7. If the device 108-7 continuously fails to meet compliance standards, the calibration report generated at step 710-7 may indicate that the device 108-7 may be dysfunctional or that calibration alone cannot solve the non-compliance issue. If the device 108-7 meets the compliance standards, the calibration module 136-7 may end. The calibration module 136-7 may schedule a time to reinitiate itself, such as once a month or every 6 months, so the device 108-7 may be recalibrated based on compliance standards. The calibration module 136-7 may reinitiate whenever an invasive device 140-7 connects to the system.

FIG. 61 illustrates an example operation of the report module 138-7. The report module 138-7 may poll, at step 800-7, for data from the calibration module 136-7. The report module 138-7 may send and/or display, at step 802-7, the received data. If a display is connected to the device 108-7 or can be reached by other means, the report module 138-7 may display the data directly. If not, the report module may send data in real-time or periodically. Data may be sent to an email address, a database, a module, or any other destination. The data may be sent to and/or displayed at multiple locations. The report module 138-7 may return, at step 804-7, to step 800-7.

In one example implementation of Embodiment 7, a method for measuring one or more analytes using a real-time, non-invasive radio frequency (“RF”) analyte detection device can include the following: a) providing the real-time, non-invasive RF analyte detection device; b) providing an invasive analyte detection device; c) comparing a first set of data from the invasive analyte detection device to a second set of data from the real-time, non-invasive RF analyte detection device; d) calibrating the real-time, non-invasive RF analyte detection device based on the compared first and second sets of data; and repeating steps c)-d) until the real-time, non-invasive RF analyte detection device meets a compliance standard.

### Embodiment 8

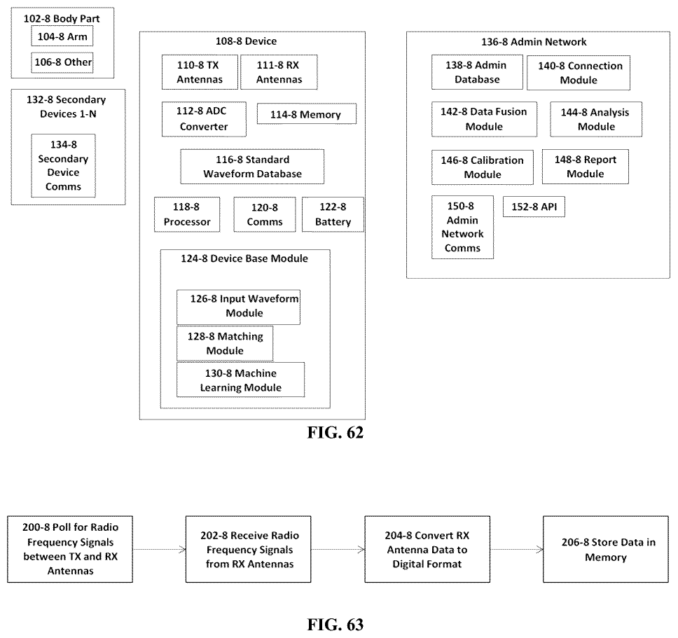

FIG. 62 is a schematic illustration of a system for radio frequency health monitoring. This system is configured to be attached to or in proximity to a body part 102-8. The body part 102-8 may be an arm 104-8. The body part 102-8 may be another body part 106-8 besides an arm, such as a leg, finger, chest, head, or any other body part from which useful medical parameters can be taken.

The system may comprise a device 108-8, which may be a wearable and portable device such as, but not limited to, a cell phone, a smartwatch, a tracker, a wearable monitor, a wristband, and a personal blood monitoring device.

The system may further comprise one or more transmit (“TX”) antennas 110-8. The one or more TX antennas 110-8 may be configured to transmit RF in the RF Activated Range from 500 MHZ to 300 GHZ. A pre-defined frequency may correspond to a range suitable for the human body in one embodiment. For example, the one or more TX antennas 110-8 would transmit radio frequency signals at a range of 120-126 GHz.

The system may further comprise one or more receive (“RX”) antennas 111-8. The one or more RX antennas 111-8 may be configured to receive the RF signals in response to the RF signal transmitted from the one or more TX antennas 110-8. The system may further comprise an ADC converter 112-8, which may be configured to convert the received RF signals from an analog signal into a digital processor readable format.

The system may further comprise memory 114-8, which may be configured to store the transmitted RF signals by the one or more TX antennas 110-8 and store the received portion of the response or responded RF signals from the one or more RX antennas 111-8. Further, the memory 114-8 may also store the converted digital processor readable format by the ADC converter 112-8. The memory 114-8 may include suitable logic, circuitry, and/or interfaces that may be configured to store a machine code and/or a computer program with at least one code section executable by the processor 118-8. Examples of implementation of the memory 114-8 may include, but are not limited to, Random Access Memory (RAM), Read Only Memory (ROM), Hard Disk Drive (HDD), and/or a Secure Digital (SD) card.

The system may further comprise a standard waveform database 116-8, which may contain standard waveforms for known patterns. These may be raw or converted device readings from patients or persons with known conditions. For example, the standard waveform database 116-8 may include raw or converted device readings from a patient known to have diabetes or an average of multiple patients. This data can be compared to readings from a person with an unknown condition in order to determine if the waveforms taken from that person match any of the known standard waveforms.

The system may further comprise a processor 118-8, which may facilitate the operation of the device 108-8 according to the instructions stored in the memory 114-8. The processor 118-8 may include suitable logic, circuitry, interfaces, and/or code that may be configured to execute a set of instructions stored in the memory 114-8.

The system may further comprise a comms 120-8, which may be a communication element such as a Wi-Fi or RFC transmitter and receiver or physical connection that can send and receive data to other devices of the system and devices external to the system. The comms 120-8 may be configured to comply with regulatory acts such as HIPPA. For example, the comms 120-8 may include features such as encryption, access control, audit trails, secure transmission, credential verification, or other features which may protect the integrity and privacy of transmitted or received data.

The system may further comprise a battery 122-8, which may power hardware modules of the device 108-8. The device 108-8 may be configured with a charging port to recharge the battery 122-8. Charging of the battery 122-8 may be wired or wireless.

The system may further comprise a device base module 124-8, which may be configured to store instructions for executing the computer program from the converted digital processor readable format of the ADC converter 112-8. The device base module 124-8 may be configured to facilitate the operation of the processor 118-8, the memory 114-8, the one or more TX antennas 110-8, the one or more RX antennas 111-8, and the comms 120-8. Further, the device base module 124-8 may be configured to create polling of the RF Activated Range from 500 MHZ to 300 GHZ. It can be noted that the device base module 124-8 may be configured to filter the RF Activated Range from 500 MHZ to 300 GHZ received from the one or more RX antennas 111-8.

The system may further comprise an input waveform module 126-8, which may extract a radio frequency waveform from memory. This may be the raw or converted recording of data from the one or more RX antennas 111-8 from a patient wearing the device. If the entire radio frequency is too long for effective matching, the input waveform module 126-8 may select a time interval within the data set. This input waveform may then be sent to the matching module 128-8.

The system may further comprise a matching module 128-8, which may match the input waveform and each the standard waveforms in the standard waveform database 116-8 by performing a convolution and/or cross-correlation of the input waveform and the standard waveform. These convolutions and/or cross-correlations are then sent to the machine learning module.

The system may further comprise a machine learning module 130-8 which has been trained to identify health parameters based on the convolution and/or cross-correlations of the input and standard waveforms. The machine learning module 130-8 receives the convolutions and cross-correlations from the matching module 128-8 and outputs any health parameters identified.

The system may further comprise one or more secondary devices 132-8, which may be a heart rate device, sleep system device, pulse rate device, or sensor inserted under the skin to measure glucose level device or any other device that produces data which may be collected by the connection module 140-8.

The system may further comprise a secondary device comms 134-8, which may be a communication element such as a Wi-Fi or RFC transmitter and receiver or physical connection that can send and receive data to other devices of the system and devices external to the system. The secondary device comms 134-8 may be configured to comply with regulatory acts such as HIPPA. For example, the secondary device comms 134-8 may include encryption, access control, audit trails, secure transmission, credential verification, or other features that may protect the integrity and privacy of transmitted or received data.

The system may further comprise an admin network 136-8, which may be a computer or network of computers which may send and receive data. The admin network 136-8 may be accessed via an application on a user device such as a PC, smartphone, smartwatch, iPhone, etc. The admin network 136-8 may be an application center or store which allows secondary devices 132-8 to be registered to communicate with the network. The admin network 136-8 may communicate with other networks, such as third-party networks or other iterations of the admin network 136-8.

The system may further comprise an admin database 138-8, which may store the health parameters output by the machine learning module 130-8 and data collected from the secondary devices 132-8.

The system may further comprise a connection module 140-8, which may connect to the device 108-8 and the secondary devices 132-8 and collect data which then may be stored in the admin database 138-8.

The system may further comprise a data fusion module 142-8, which may fuse the real-time analyte data from the device 108-8 and collected data from the secondary devices 132-8 stored in the admin database 138-8. Fusing data may refer to the process of combining multiple sources of information or data sets into a single integrated output. This process involves aligning and combining data from different sources while ensuring that it remains accurate and consistent. For example, fusing data from the non-invasive RF analyte detection device and other physiological devices, such as heart rate, sleep system, or pulse rate devices, would enable the identification of correlations or patterns that may exist between the analyte levels and other physiological variables. Such information can be useful in providing a more comprehensive understanding of the individual's health status and may aid in the development of targeted interventions or treatments.

The system may further comprise an analysis module 144-8, which runs analytic models such as artificial intelligence (AI) and machine learning (ML) algorithms on the data in the admin database 138-8 and/or fused data from the data fusion module 142-8. AI and/or ML algorithms, contextual analysis, and predictive analytics are employed to identify patterns, provide personalized insights, and forecast future health outcomes using data. Real-time monitoring offers immediate feedback on aspects like hydration, while integration with other health data creates a comprehensive view of a user's well-being. The system may further comprise a calibration module 146-8, which may calibrate the device 108-8 and/or less accurate secondary devices 132-8 based on the collected data real-time analyte data from more accurate secondary devices 132-8 admin database 138-8. More accurate secondary devices 132-8 may be devices that measure health parameters with higher accuracy, such as invasive devices, medical grade devices, wired devices, full-body devices, etc.

The calibration module 146-8 may be run continuously for initial calibration and then periodically to check that the device 108-8 is still calibrated. The system may further comprise a report module 148-8, which may display a report of the data from the analysis module 144-8. This report may be sent to an email address, displayed on a screen, sent to another module, or otherwise reported.

The system may further comprise an admin network comms 150-8, which may be a communication element such as a Wi-Fi or RFC transmitter and receiver or physical connection that can send and receive data to other devices of the system and devices external to the system. The admin network comms 150-8 may be configured to comply with regulatory acts such as HIPPA. For example, the admin network comms 150-8 may include encryption, access control, audit trails, secure transmission, credential verification, or other features that may protect the integrity and privacy of transmitted or received data.

The system may further comprise an application programming interface or API 152-8, which is a set of definitions and protocols for building and integrating application software. The API 152-8 may be used by programs that want to communicate with the admin network 136-8. The API 152-8 may be used to prompt a third-party application. The third-party application may be a large AI or ML system for data analysis, for example, a natural language model such as GPT-4.

FIG. 63 illustrates an example operation of the device base module 124-8. The process may begin with the device base module 124-8 polling the Active Range RF signals between the one or more TX antennas 110-8 and the one or more RX antennas 111-8 at step 200-8. The device base module 124-8 may be configured to read and process instructions stored in the memory 114-8 using the processor 118-8. The one or more TX antennas 110-8 may be configured to transmit RF in the RF Activated Range from 500 MHZ to 300 GHZ. For example, the one or more TX antennas 110-8 may use RF signals at a range of 500 MHZ to 300 GHZ. The device base module 124-8 may receive the RF frequency signals from the one or more RX antennas 111-8 at step 202-8. For example, one or more RX antennas 111-8 receives a response or responded RF of frequency range 300-330 GHz from the patient's blood. The device base module 124-8 may be configured to convert the received RF signals into a digital format using the ADC 112-8 at step 204-8. For example, the received RF of 300-330 GHz frequency range is converted into a 10-bit data signal. The device base module 124-8 may be configured to store converted digital format into the memory 114-8 at step 206-8.

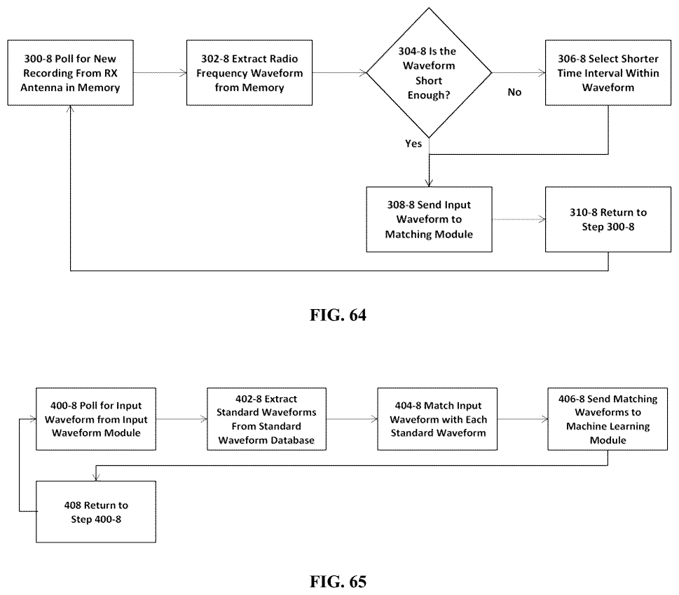

FIG. 64 illustrates an example operation of the input waveform module 126-8. The process may begin with the input waveform module 126-8 polling, at step 300-8, for new recorded data from the one or more RX antennas 111-8 stored in memory 114-8. The input waveform module 126-8 may extract, at step 302-8, the recorded radio frequency waveform from memory. If there is more than one waveform recorded, the input waveform module 126-8 may select each waveform separately and loop through the following steps. The input waveform module 126-8 may determine, at step 304-8, if the waveform is small enough to be an input waveform for the matching module 128-8. This will depend on the computational requirements and/or restrictions of the matching module 128-8. If the waveform is short enough, the input waveform module 126-8 may skip to step 308-8. If the waveform is too long, the input waveform module 126-8 may select, at step 306-8, a shorter time interval within the entire recorded waveform. For example, if the waveform is 5 minutes long, then only a 30 second interval may be selected. The interval may be selected at random or by a selection process. The input waveform module 126-8 may send, at step 308-8, the input waveform to the matching module 128-8. The input waveform module 126-8 may return, at step 310-8, to step 300-8.

FIG. 65 illustrates an example operation of the matching module 128-8. The process may begin with the matching module 128-8 polling, at step 400-8, for an input waveform from the input waveform module 126-8. The matching module 128-8 may extract, at step 402-8, each standard waveform from the standard waveform database 116-8. The matching module 128-8 may match, at step 404-8, the input waveform with each standard waveform. Matching may be determining which standard waveforms the input waveform is similar to. Matching may involve convolution and/or cross-correlation of the waveforms or any other matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function whose values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-8. The matching module 128-8 may send, at step 406-8, the matching waveforms to the machine learning module 130-8. Matching waveforms may refer to the standard waveforms similar to the input waveform, the waveforms generated via convolution and/or cross-correlation, or both. The matching module 128-8 may return, at step 408-8, to step 400-8.

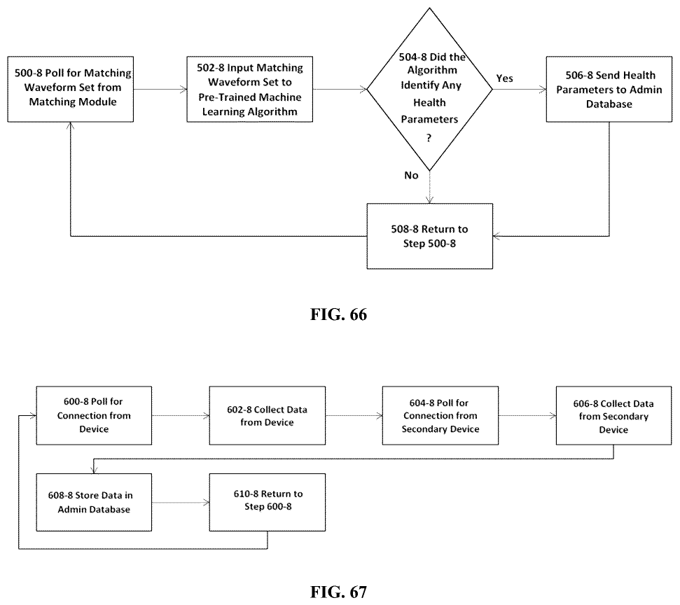

FIG. 66 illustrates an example operation of the machine learning module 130-8. The process may begin with the machine learning module 130-8 polling, at step 500-8, for a set of matching waveforms from the matching module 128-8. Matching waveforms may be a set of standard waveforms similar to the input waveform or statistical combinations of the input waveform and standard waveforms, such as convolutions or cross-correlations. The machine learning module 130-8 may input, at step 502-8, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During the training process, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-8 may determine, at step 504-8, if the algorithm identified any health parameters. Identification may require a certain interval of confidence. For example, if the machine learning algorithm determines that it is more than 70% likely that a health parameter is correct, that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130-8 may skip to step 508-8. If any health parameters were identified, the machine learning module 130-8 may send, at step 506-8, the health parameters to the admin database 138-8. The machine learning module 130-8 may return, at step 508-8, to step 500-8.

FIG. 67 illustrates an example operation of the connection module 140-8. The connection module 140-8 may poll, at step 600-8, for a connection from the device 108-8. If no connection is detected the connection module 140-8 may search for a device 108-8 that is attempting to connect or may skip to step 604-8. The connection module 140-8 may collect, at step 602-8, data from the device 108-8. This may be real-time data or periodically updated data. This data may come directly from the machine learning module 130-8. The connection module 140-8 may poll, at step 604-8, for a connection from one or more secondary devices 132-8. If no connection is detected the connection module 140-8 may search for a secondary device 132-8 that is attempting to connect or may skip to step 608-8. The connection module 140-8 may begin at this step if a secondary device 132-8 is unavailable or unnecessary. The connection module 140-8 may collect, at step 606-8, data from the secondary devices 132-8. This may be real-time data or periodically updated data. The connection module 140-8 may store, at step 608-8, the data from the device 108-8 and/or secondary device 132-8 in the admin database 138-8. The connection module 140-8 may add contextual data such as the ID of the secondary device 132-8, the ID of the device 108-8, the type of secondary device 132-8, the analyte measured by the device 108-8, the time data was retrieved, etc., to the received data before storage in the admin database 138-8. The connection module 140-8 may return, at step 610-8, to step 600-8.

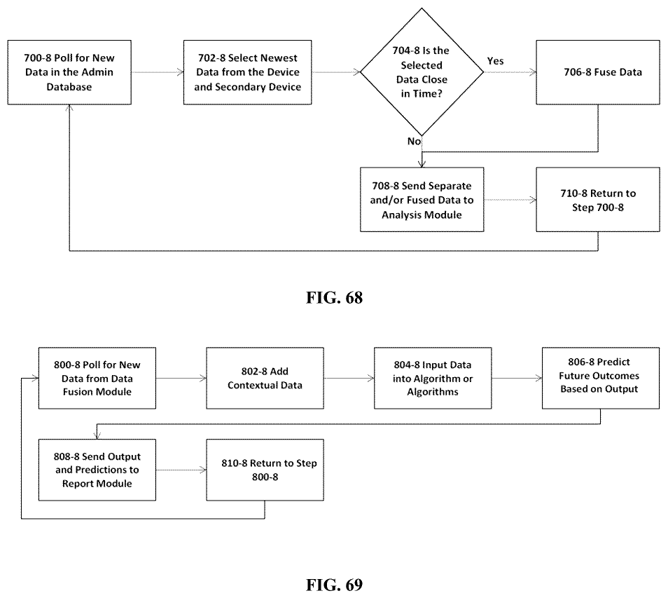

FIG. 68 illustrates an example operation of the data fusion module 142-8. The data fusion module 142-8 may poll, at step 700-8, for new data in the admin database 138-8. This new data may be from device 108-8 or one or more secondary devices 132-8. The data fusion module 142-8 may select, at step 702-8, the newest data from the device 108-8 and the newest data from each secondary device 132-8. This may be a single data point or a set of data. The data fusion module 142-8 may determine, at step 704-8, if the selected data is close in time. Close in time may mean each piece or set of data is timestamped within a few seconds of each other. For example, the newest data from the device 108-8 may be real-time, but the newest reading from a secondary device 132-8 may have been 10 minutes ago. This data would not be close in time if the threshold is a few seconds. The threshold may be varied based on the health parameter being measured. For example, if a secondary device 132-8 is recording sleep cycle activity, then recordings from 10 minutes ago may be relevant because of the gradual nature of sleep cycles, but if a secondary device 132-8 is recording blood glucose or electrolyte levels, then data from minutes ago may be highly inaccurate. The time threshold for what data is considered close in time may be set by a user of the device, manufacturer of the device, and/or another module. If the selected data is not close in time, the data fusion module 142-8 may skip to step 708-8. The data fusion module 142-8 may fuse, at step 706-8, the selected data from the device 108-8 and secondary devices 132-8. Fusion of data may describe multiple processes, and which process is performed may depend on the types of data being compared. For example, if both pieces or data sets are measurements of the same parameter, such as glucose, then fusing the data may refer to aligning and combining data to ensure it is accurate and consistent. Aligning may be taking the mean or the median. The data may be enhanced, meaning an error is returned if both measurements are outside a predetermined normal range. For another example, fused data may be a correlation function of two sets of data, such as correlating real-time glucose measurements from the device 108-8 to sleep cycle readings from a secondary device 132-8 in order to track the effect of blood glucose on sleep quality. Fusing the data may refer to adjusting one piece or set of data based on the other. For example, pulse rate readings from a secondary device 132-8 may be used to adjust SP02 readings from the device 108-8. which are calibrated for a normal pulse rate. Fusing the data may refer to generating additional health parameters from the combination of the data. For example, blood glucose measurements from the device 108-8, blood cortisol levels from a secondary device 132-8, and heart rate measurements from another secondary device 132-8 can be fused to generate stress or activity level. Fusing data may refer to estimating a risk level based on the data sets. For example, blood glucose data from the device 108-8, heart rate data from a secondary device 132-8, and tissue oxygenation data from another secondary device 132-8 may all be factored into a risk score. The data fusion module 142-8 may use statistical algorithms, machine learning, and/or AI to perform the data fusion. The data fusion module 142-8 may send, at step 708-8, each piece or set of data and/or the fused data, if available, to the analysis module 144-8. The data fusion module 142-8 may return, at step 710-8, to step 700-8.

FIG. 69 illustrates an example operation of the analysis module 144-8. The analysis module 144-8 may poll, at step 800-8, for data from the data fusion module 142-8. This data may include fused data and unfused data. The analysis module 144-8 may add to, at step 802-8, the collected data by incorporating contextual information such as the patient's age, sex, weight, location, time of day, activity level, etc., if that information is available. For example, the system could combine heart rate data with information about the patient's location and exercise intensity to better understand the impact of their workout on their health. The analysis module 144-8 may incorporate additional contextual information such as medical history, genetic predispositions, or environmental factors to enhance the collected data. For example, the system could combine blood pressure data with the patient's age, family history of hypertension, and current stress levels to better understand their cardiovascular risk profile. This data may be supplied by the patient or their healthcare providers. This data may be stored in the admin database 138-8 or retrieved from another database external to the admin network 136-8 such as one containing the patient's medical history or a database on the patient's personal device such as a smartphone. The analysis module 144-8 may also integrate data from wearable devices or mobile apps, providing real-time updates on the patient's lifestyle habits, such as diet and exercise routines, further enhancing the quality of the analysis. The analysis module 144-8 may input, at step 804-8, the new data, along with any contextual information, into one or more trained machine learning or AI algorithms. For example, the system could input patients' heart rate, exercise intensity, and location data to generate personalized insights about their cardiovascular health. The contextual data may influence the output. For example, a young obese patient with a fasting blood glucose level of 130 mg/dL may have outputs related to pre-diabetes, whereas the same blood glucose levels in an older, thinner patient may have outputs related to liver disease. The analysis module 144-8 may input the collected data and contextual information into multiple trained machine learning or AI algorithms, each specialized in analyzing specific aspects of the patient's health. For example, the system could input sleep pattern data and environmental factors into a dedicated sleep analysis algorithm while simultaneously inputting exercise data and genetic predispositions into a separate algorithm focused on physical fitness assessment. The analysis module 144-8 may also identify potential correlations between different health outcomes, enabling healthcare providers to simultaneously address the root cause of multiple health issues. The analysis module 144-8 may use external algorithms, such as GPT-4, through the use of the API 152-8. The analysis module 144-8 may use, at step 806-8, the trained predictive analytics algorithm to forecast future health outcomes based on the output from the algorithm or algorithms and/or the input data. For example, the system could predict the likelihood of a patient developing obesity or diabetes by analyzing trends in their physical activity, diet, and sleep patterns. This information could then be used to provide targeted interventions, such as personalized exercise plans or nutritional guidance, to help mitigate the risk of developing these health conditions. The analysis module 144-8 may utilize multiple predictive analytics algorithms to forecast future health outcomes for various aspects of the patient's well-being. Based on the outputs from multiple specialized algorithms, the analysis module 144-8 may predict the patient's risk of developing cardiovascular diseases, mental health disorders, or age-related conditions. For example, the output of the algorithm or algorithms in step 804-8 may indicate pre-diabetes and poor sleep quality, which may result in the predictive analytics algorithm's prediction that the patient is at high risk of early onset Alzheimer's disease. These predictions could be combined to provide a holistic view of the patient's overall health status, enabling healthcare providers to design comprehensive, personalized intervention strategies addressing multiple health concerns. The analysis module 144-8 may also consider the patient's personal preferences, such as dietary restrictions or exercise preferences, to ensure that the generated insights are tailored to the individual and practical for implementation. The analysis module 144-8 may send, at step 808-8, the output data from the algorithm or algorithms in step 804-8 and/or the output of the predictive analytics algorithm in step 806-8 to the report module 148-8. The analysis module 144-8 may send the output data in a format that is easily understandable by the patient and healthcare providers, facilitating clear communication and collaboration for improving the patient's overall health. The analysis module 144-8 may return, at step 810-8, to step 800-8.

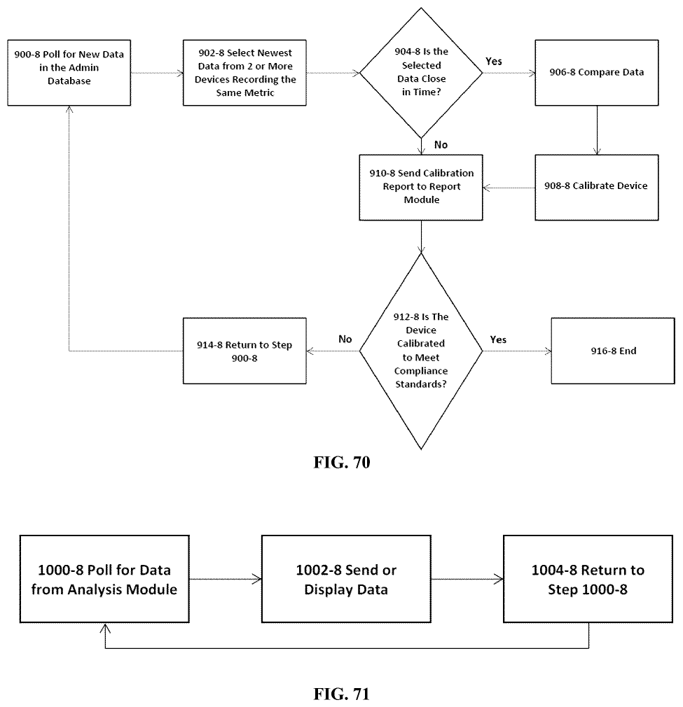

FIG. 70 illustrates an example operation of the calibration module 146-8. The calibration module 146-8 may poll, at step 900-8, for new data in the admin database 138-8. This new data may be data from the device 108-8 and/or from the secondary devices 132-8. The calibration module 146-8 may select, at step 902-8, the newest data from the device 108-8, the newest data from any 2 or more devices, including the device 108-8, and any secondary devices 132-8, which are recording the same metric. For example, the device 108-8 and two secondary devices 132-8 may all be recording blood glucose levels. The calibration module 146-8 may determine, at step 904-8, if the selected data is close in time. Close in time may mean each piece or set of data is timestamped within a few seconds of each other. For example, the newest data from the device 108-8 may be real time but the newest reading from a secondary device 132-8 may have been 10 minutes ago. This data would not be close in time if the threshold is a few seconds. The threshold may be varied based on the health parameter being measured. For example, if a secondary device 132-8 is recording slowly changing analytes such as platelet count, then recordings from 10 minutes ago may be relevant because of the gradual nature of sleep cycles, but if a secondary device 132-8 is recording blood glucose or electrolyte levels, then data from minutes ago may be highly inaccurate. The time threshold for what data is considered close in time may be set by a device user, manufacturer of the device, and/or another module. If all of the selected data is not closed in time, the calibration module 146-8 may skip to step 910-8 and report a failure to calibrate. The calibration module 146-8 may compare, at step 906-8, each device's data. This comparison helps to assess the device's accuracy and identify any discrepancies between the sets of measurements, the calibration module 146-8 may utilize statistical techniques for comparing time-synchronized data obtained from each device. These techniques may include error analysis methods, such as calculating the Mean Absolute Error (MAE) or Root Mean Square Error (RMSE) between corresponding data points, as well as regression analysis methods, such as fitting a linear regression model to the data and evaluating the coefficient of determination (R2) to assess the degree of agreement between the two data sets. By employing these comparison methods, the system can identify discrepancies or trends in the data, enabling necessary adjustments to the algorithms to enhance the accuracy and reliability of the non-invasive RF sensor system. The calibration module 146-8 may wait until enough time-synchronized data points are obtained from the device 108-8 and secondary devices 132-8 to perform a comprehensive comparison. These datapoints may be temporarily marked in the admin database 138-8 or stored in a separate database. The calibration module 146-8 may adjust, at step 908-8, the less accurate devices based on the recordings from the more accurate devices. For example, suppose the device 108-8, an external secondary device 132-8, and an internal secondary device 132-8 all recorded blood glucose levels. In that case, the device 108-8 and external secondary device 132-8 may be calibrated to closer match the internal secondary device 132-8 because the internal readings are presumed to be more accurate. Which devices are the most accurate may be indicated by an administrator of the network, found by comparing the specifications of each device from the manufacturer, or learned over time using a machine learning algorithm. The calibration module 146-8 may employ adaptive learning techniques to continuously refine the calibration process as more data is collected. This feature allows the system to improve its performance over time, adapting to the patient's unique physiology and changes in their health condition. The calibration module 146-8 may generate, at step 910-8, reports on the success or failure of the calibration process, as well as the degree of improvement achieved. These reports may be sent to the report module 148-8. These reports can be sent to an email address, displayed on a screen, forwarded to another module, or otherwise communicated to the user or healthcare provider. The calibration module 146-8 may determine, at step 912-8, if the device 108-8 is calibrated to meet compliance standards. For example, compliance standards may require the device 108-8 to give results within 0.5% of the results given by a secondary device 132-8. If the results from the device 108-8 are outside of this range with a set time period, such as over 24 hours, then the device 108-8 may not be compliant and may require further calibration or recalibration. Compliance standards may refer to legal standards, industry standards, standards set by the patient or their healthcare professionals, standards set by the manufacturer, etc. If multiple standards are used, the strictest standard may be used. If the device 108-8 does not meet compliance standards, the calibration module 146-8 may return, at step 914-8, to step 900-8. If the device 108-8 continuously fails to meet compliance standards, the calibration report generated at step 910-8 may indicate that the device 108-8 may be dysfunctional or that calibration alone cannot solve the non-compliance issue. If the device 108-8 meets the compliance standards, the calibration module 146-8 may end at step 916-8. The calibration module 146-8 may schedule a time to reinitiate itself, such as once a month or every 6 months, so that the device 108-8 may be recalibrated based on compliance standards. The calibration module 146-8 may reinitiate anytime a secondary device 132-8 with higher accuracy than the device 108-8 is connected to the system.

FIG. 71 illustrates an example of the report module 148-8. The report module 148-8 may poll, at step 1000-8, for data from the analysis module 144-8. If the calibration module 146-8 calibrated an instrument, the calibration report may also be obtained at this step. The report module 148-8 may send and/or display, at step 1002-8, the received data. If a display is connected to the admin network 136-8 or can be reached by other means, the report module 148-8 may display the data directly. If not, the report module may send data in real-time or periodically. Data may be sent to an email address, a database, a module, or any other destination. The data may be sent to and/or displayed at multiple locations. The report module 148-8 may return, at step 1004-8, to step 1000-8.

In one example implementation of Embodiment 8, a system for measuring one or more analytes using a real-time, non-invasive radio frequency (“RF”) analyte detection device can include: the real-time, non-invasive RF analyte detection device; at least one secondary device for recording health parameters; and an admin network; wherein data that is collected from the non-invasive RF analyte detection device and the at least one secondary device is stored on the admin network; and wherein at least one of the real-time, non-invasive RF analyte detection device and the at least one secondary device is calibrated based on the data from at least one of the real-time, non-invasive RF analyte detection device and the at least one secondary device; and wherein the data from two or more of the real-time, non-invasive RF analyte detection device and the at least one secondary device is analyzed using a data analysis algorithm; and wherein the analyzed data is reported to at least one user of the system.

### Embodiment 9

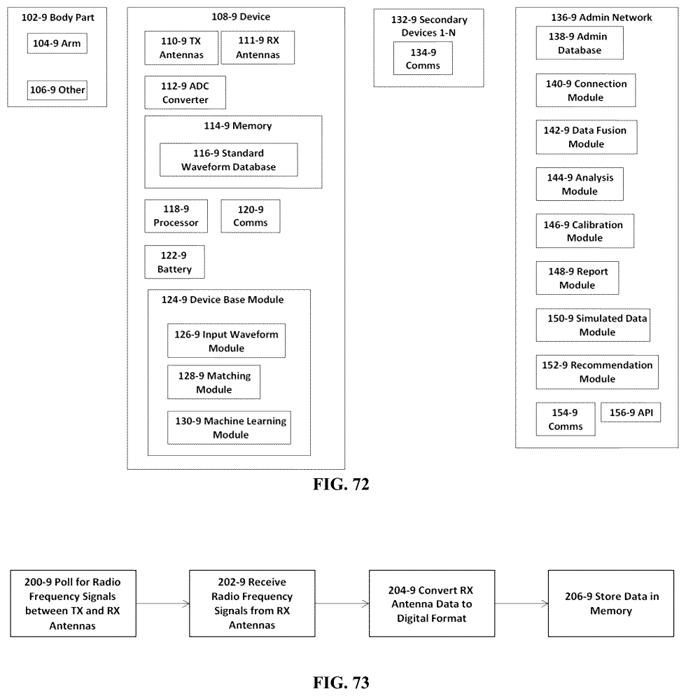

FIG. 72 is a schematic illustration of a system for radio frequency health monitoring. This system is configured to be attached to or in proximity to a body part 102-9. The body part 102-9 may be an arm 104-9. The body part 102-9 may be another body part 106-9 besides an arm, such as a leg, finger, chest, head, or any other body part from which useful medical parameters can be taken.

The system may comprise a device 108-9, which may be a wearable and portable device such as, but not limited to, a cell phone, a smartwatch, a tracker, a wearable monitor, a wristband, and a personal blood monitoring device.

The system may further comprise one or more transmit (“TX”) antennas 110-9. The one or more TX antennas 110-9 may be configured to transmit RF in the RF Activated Range from 500 MHZ to 300 GHZ. A pre-defined frequency may correspond to a range suitable for the human body in one embodiment. For example, the one or more TX antennas 110-9 would transmit radio frequency signals at a range of 120-126 GHz.

The system may further comprise one or more receive (“RX”) antennas 111-9. The one or more RX antennas 111-9 may be configured to receive the RF signals in response to the RF signal transmitted from the one or more TX antennas 110-9.

The system may further comprise an ADC converter 112-9, which may be configured to convert the received RF signals from an analog signal into a digital processor readable format.

The system may further comprise memory 114-9, which may be configured to store the transmitted RF signals by the one or more TX antennas 110-9 and store the received portion of the response or responded RF signals from the one or more RX antennas 111-9. Further, the memory 114-9 may also store the converted digital processor readable format by the ADC converter 112-9. The memory 114-9 may include suitable logic, circuitry, and/or interfaces that may be configured to store a machine code and/or a computer program with at least one code section executable by the processor 118-9. Examples of implementation of the memory 114-9 may include, but are not limited to, Random Access Memory (RAM), Read Only Memory (ROM), Hard Disk Drive (HDD), and/or a Secure Digital (SD) card.

The system may further comprise a standard waveform database 116-9, which may contain standard waveforms for known patterns. These may be raw or converted device readings from patients or persons with known conditions. For example, the standard waveform database 116-9 may include raw or converted device readings from a patient known to have diabetes or an average of multiple patients. This data can be compared to readings from a person with an unknown condition in order to determine if the waveforms taken from that person match any of the known standard waveforms.

The system may further comprise a processor 118-9, which may facilitate the operation of the device 108-9 according to the instructions stored in the memory 114-9. The processor 118-9 may include suitable logic, circuitry, interfaces, and/or code that may be configured to execute a set of instructions stored in the memory 114-9.

The system may further comprise a comms 120-9, which may be a communication element such as a Wi-Fi or RFC transmitter and receiver or physical connection that can send and receive data to other devices of the system and devices external to the system. The comms 120-9 may be configured to comply with regulatory acts such as HIPPA. For example, the comms 120-9 may include features such as encryption, access control, audit trails, secure transmission, credential verification, or other features which may protect the integrity and privacy of transmitted or received data.

The system may further comprise a battery 122-9, which may power hardware modules of the device 108-9. The device 108-9 may be configured with a charging port to recharge the battery 122-9. Charging of the battery 122-9 may be wired or wireless.

The system may further comprise a device base module 124-9, which may be configured to store instructions for executing the computer program from the converted digital processor readable format of the ADC converter 112-9. The device base module 124-9 may be configured to facilitate the operation of the processor 118-9, the memory 114-9, the one or more TX antennas 110-9, the one or more RX antennas 111-9, and the comms 120-9. Further, the device base module 124-9 may be configured to create polling of the RF Activated Range from 500 MHZ to 300 GHZ. It can be noted that the device base module 124-9 may be configured to filter the RF Activated Range from 500 MHZ to 300 GHZ received from one or more RX antennas 111-9.

The system may further comprise an input waveform module 126-9, which may extract a radio frequency waveform from memory. This may be the raw or converted recording of data from the one or more RX antennas 111-9 from a patient wearing the device. If the entire radio frequency is too long for effective matching, the input waveform module 126-9 may select a time interval within the data set. This input waveform may then be sent to the matching module 128-9.

The system may further comprise a matching module 128-9, which may match the input waveform and each of the standard waveforms in the standard waveform database 116-9 by performing a convolution and/or cross-correlation of the input waveform and the standard waveform. These convolutions and/or cross-correlations are then sent to the machine learning module.

The system may further comprise a machine learning module 130-9 which has been trained to identify health parameters based on the convolution and/or cross-correlations of the input and standard waveforms. The machine learning module 130-9 receives the convolutions and cross-correlations from the matching module 128-9 and outputs any health parameters identified.

The system may further comprise one or more secondary devices 132-9, which may be a heart rate device, sleep system device, pulse rate device, or sensor inserted under the skin to measure glucose level, or any other device that produces data which may be collected by the connection module 140-9.

The system may further comprise a secondary device comms 134-9, which may be a communication element such as a Wi-Fi or RFC transmitter and receiver or physical connection that can send and receive data to other devices of the system and devices external to the system. The secondary device comms 134-9 may be configured to comply with regulatory acts such as HIPPA. For example, the secondary device comms 134-9 may include encryption, access control, audit trails, secure transmission, credential verification, or other features that may protect the integrity and privacy of transmitted or received data.

The system may further comprise an admin network 136-9, which may be a computer or network of computers which may send and receive data. The admin network 136-9 may be accessed via an application on a user device such as a PC, smartphone, smartwatch, iPhone, etc. The admin network 136-9 may be an application center or store which allows secondary devices 132-9 to be registered to communicate with the network. The admin network 136-9 may communicate with other networks, such as third-party networks or other iterations of the admin network 136-9.

The system may further comprise an admin database 138-9, which may store the health parameters output by the machine learning module 130-9 and data collected from the secondary devices 132-9.

The system may further comprise a connection module 140-9, which may connect to the device 108-9 and the secondary devices 132-9 and collect data which then may be stored in the admin database 138-9.

The system may further comprise a data fusion module 142-9, which may fuse the real-time analyte data from the device 108-9 and collected data from the secondary devices 132-9 stored in the admin database 138-9. Fusing data may refer to the process of combining multiple sources of information or data sets into a single integrated output. This process involves aligning and combining data from different sources while ensuring that it remains accurate and consistent. For example, fusing data from the non-invasive RF analyte detection device and other physiological devices, such as heart rate, sleep system, or pulse rate devices, would enable the identification of correlations or patterns that may exist between the analyte levels and other physiological variables. Such information can be useful in providing a more comprehensive understanding of the individual's health status and may aid in the development of targeted interventions or treatments.

The system may further comprise an analysis module 144-9, which runs analytic models such as artificial intelligence (AI) and machine learning (ML) algorithms on the data in the admin database 138-9 and/or fused data from the data fusion module 142-9. AI and/or ML algorithms, contextual analysis, and predictive analytics are employed to identify patterns, provide personalized insights, and forecast future health outcomes using data. Real-time monitoring offers immediate feedback on aspects like hydration, while integration with other health data creates a comprehensive view of a user's well-being.

The system may further comprise a calibration module 146-9, which may calibrate the device 108-9 and/or less accurate secondary devices 132-9 based on the collected real-time analyte data from more accurate secondary devices 132-9 stored in the admin database 138-9. More accurate secondary devices 132-9 may be devices that measure health parameters with higher accuracy, such as invasive devices, medical grade devices, wired devices, full-body devices, etc. The calibration module 146-9 may be run continuously for initial calibration and then run periodically to check that the device 108-9 is still calibrated.

The system may further comprise a report module 148-9, which may display a report of the data from the analysis module 144-9. This report may be sent to an email address, displayed on a screen, sent to another module, or otherwise reported.

The system may further comprise a simulated data module 150-9, which may select simulated additional data to add to the most recent set of input data in the admin database 138-9. The simulated data module 150-9 then determines if the additional data would increase the confidence of the final output or outputs of the analysis module 144-9. The simulated data module 150-9 then sends the simulated data type that results in the highest confidence level to the recommendation module 152-9. The recommendation module 152-9 may then recommend actually taking those measurements. For example, the original set of input data includes data from the device 108-9 recording blood glucose levels and a blood pressure secondary device 132-9. After fusion and analysis, the output of this set of data is a blood glucose level of 114 mg/dL, with a confidence interval of 85%. Based on simulations with additional data, the confidence interval could be raised to a maximum of 96% if the patient's blood oxygen levels were 97%. This data is sent to the recommendation module 152-9 so that it may recommend taking blood oxygen measurements.

The system may further comprise a recommendation module 152-9, which may recommend measuring additional metrics based on the results of the simulated data module 150-9.

The system may further comprise an admin network comms 154-9, which may be a communication element such as a Wi-Fi or RFC transmitter and receiver or physical connection that can send and receive data to other devices of the system and devices external to the system. The admin network comms 154-9 may be configured to comply with regulatory acts such as HIPPA. For example, the admin network comms 154-9 may include features such as encryption, access control, audit trails, secure transmission, credential verification, or other features that may protect the data's integrity and privacy.

The system may further comprise an application programming interface or API 156-9, which is a set of definitions and protocols for building and integrating application software. The API 156-9 may be used by programs that want to communicate with the admin network 136-9. The API 156-9 may be used to prompt a third-party application. The third-party application may be a large AI or ML system for data analysis, for example, a natural language model such as GPT-4.

FIG. 73 illustrates an example operation of the device base module 124-9. The process may begin with the device base module 124-9 polling the Active Range RF signals between the one or more TX antennas 110-9 and the one or more RX antennas 111-9 at step 200-9. The device base module 124-9 may be configured to read and process instructions stored in the memory 114-9 using the processor 118-9. The one or more TX antennas 110-9 may be configured to transmit RF in the RF Activated Range from 500 MHZ to 300 GHZ. For example, the one or more TX antennas 110-9 may use RF signals at a range of 500 MHZ to 300 GHZ. The device base module 124-9 may receive the RF frequency signals from the one or more RX antennas 111-9 at step 202-9. For example, one or more RX antennas 111-9 receives a response or responded RF of frequency range 300-330 GHz from the patient's blood. The device base module 124-9 may be configured to convert the received RF signals into a digital format using the ADC 112-9 at step 204-9. For example, the received RF of 300-330 GHz frequency range is converted into a 10-bit data signal. The device base module 124-9 may be configured to store converted digital format into the memory 114-9 at step 206-9.

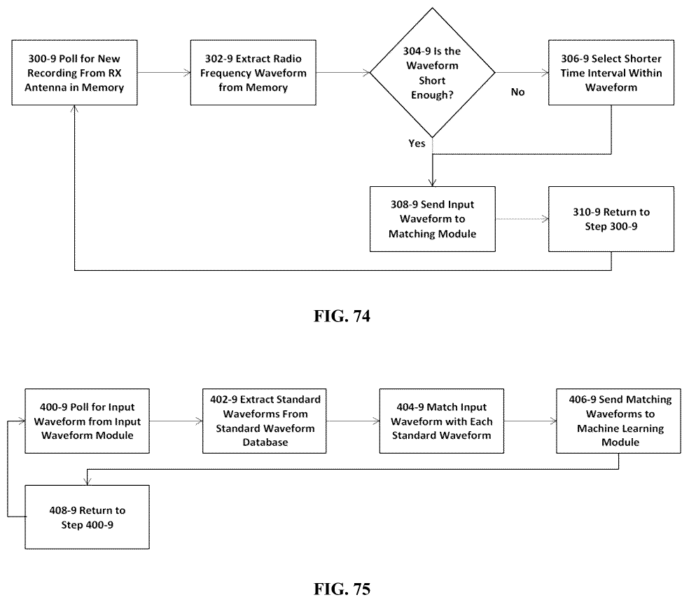

FIG. 74 illustrates an example operation of the input waveform module 126-9. The process may begin with the input waveform module 126-9 polling, at step 300-9, for new recorded data from the one or more RX antennas 111-9 stored in memory 114-9. The input waveform module 126-9 may extract, at step 302-9, the recorded radio frequency waveform from memory. If there is more than one waveform recorded, the input waveform module 126-9 may select each waveform separately and loop through the following steps. The input waveform module 126-9 may determine, at step 304-9, if the waveform is small enough to be an input waveform for the matching module 128-9. This will depend on the computational requirements and/or restrictions of the matching module 128-9. If the waveform is short enough, the input waveform module 126-9 may skip to step 308-9. If the waveform is too long, the input waveform module 126-9 may select, at step 306-9, a shorter time interval within the entire recorded waveform. For example, if the waveform is 5 minutes long, then only a 30-second interval may be selected. The interval may be selected at random or by a selection process. The input waveform module 126-9 may send, at step 308-9, the input waveform to the matching module 128-9. The input waveform module 126-9 may return, at step 310-9, to step 300-9.

FIG. 75 illustrates an example operation of the matching module 128-9. The process may begin with the matching module 128-9 polling, at step 400-9, for an input waveform from the input waveform module 126-9. The matching module 128-9 may extract, at step 402-9, each standard waveform from the standard waveform database 116-9. The matching module 128-9 may match, at step 404-9, the input waveform with each standard waveform. Matching may be determining which standard waveforms the input waveform is similar to. Matching may involve convolution and/or cross-correlation of the waveforms or any other matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a value of similarity between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function whose values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-9. The matching module 128-9 may send, at step 406-9, the matching waveforms to the machine learning module 130-9. Matching waveforms may refer to the standard waveforms similar to the input waveform, the waveforms generated via convolution and/or cross-correlation, or both. The matching module 128-9 may return, at step 408-9, to step 400-9.

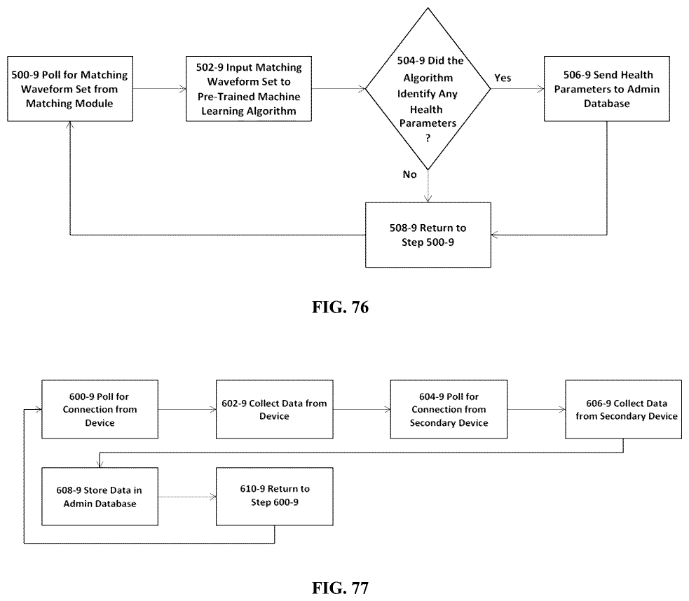

FIG. 76 illustrates an example operation of the machine learning module 130-9. The process may begin with the machine learning module 130-9 polling, at step 500-9, for a set of matching waveforms from the matching module 128-9. Matching waveforms may be a set of standard waveforms that are similar to the input waveform or statistical combinations of the input waveform and standard waveforms, such as convolutions or cross-correlations. The machine learning module 130-9 may input, at step 502-9, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During the training process, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-9 may determine, at step 504-9, if the algorithm identified any health parameters. Identification may require a certain interval of confidence. For example, if the machine learning algorithm determines that it is more than 70% likely that a health parameter is correct, that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130-9 may skip to step 508-9. If any health parameters were identified, the machine learning module 130-9 may send, at step 506-9, the health parameters to the admin database 138-9. The machine learning module 130-9 may return, at step 508-9, to step 500-9.

FIG. 77 illustrates an example operation of the connection module 140-9. The connection module 140-9 may poll, at step 600-9, for a connection from the device 108-9. If no connection is detected, the connection module 140-9 may search for a device 108-9 attempting to connect or skip to step 604-9. The connection module 140-9 may collect, at step 602-9, data from the device 108-9. This may be real-time data or periodically updated data. This data may come directly from the machine learning module 130-9. The connection module 140-9 may poll, at step 604-9, for a connection from one or more secondary devices 132-9. If no connection is detected, the connection module 140-9 may search for a secondary device 132-9 attempting to connect or skip to step 608-9. The connection module 140-9 may begin at this step if a secondary device 132-9 is unavailable or unnecessary. The connection module 140-9 may collect, at step 606-9, data from the secondary devices 132-9. This may be real-time data or periodically updated data. The connection module 140-9 may store, at step 608-9, the data from the device 108-9 and/or secondary device 132-9 in the admin database 138-9. The connection module 140-9 may add contextual data such as the ID of the secondary device 132-9, the ID of the device 108-9, the type of secondary device 132-9, the analyte measured by the device 108-9, the time data was retrieved, etc., to the received data before storage in the admin database 138-9. The connection module 140-9 may return, at step 610-9, to step 600-9.

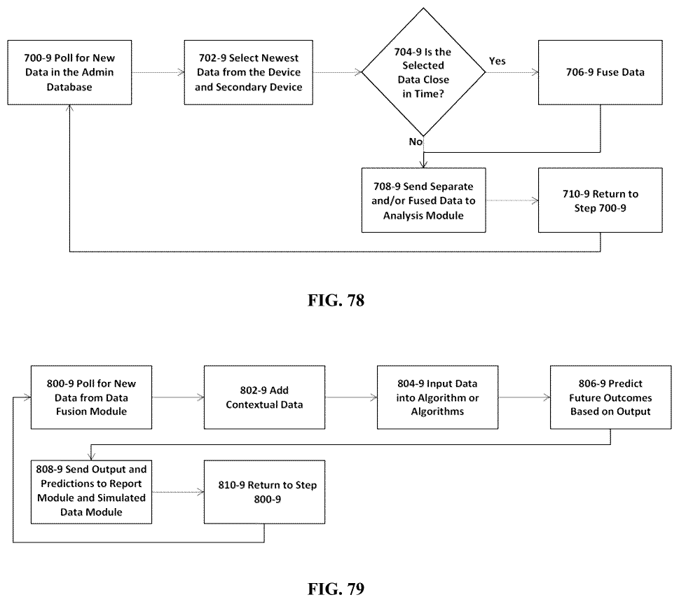

FIG. 78 illustrates an example operation of the data fusion module 142-9. The data fusion module 142-9 may poll, at step 700-9, for new data in the admin database 138-9. This new data may be from device 108-9 and/or one or more secondary devices 132-9. The data fusion module 142-9 may select, at step 702-9, the newest data from the device 108-9 and the newest data from each secondary device 132-9. This may be a single data point or a set of data. The data fusion module 142-9 may determine, at step 704-9, if the selected data is close in time. Close in time may mean each piece or set of data is timestamped within a few seconds of each other. For example, the newest data from the device 108-9 may be real-time, but the newest reading from a secondary device 132-9 may have been 10 minutes ago. This data would not be close in time if the threshold is a few seconds. The threshold may be varied based on the health parameter being measured. For example, if a secondary device 132-9 is recording sleep cycle activity, then recordings from 10 minutes ago may be relevant because of the gradual nature of sleep cycles, but if a secondary device 132-9 is recording blood glucose or electrolyte levels, then data from minutes ago may be highly inaccurate. The time threshold for what data is considered close in time may be set by a user of the device, manufacturer of the device, and/or another module. If the selected data is not close in time, the data fusion module 142-9 may skip to step 708-9. The data fusion module 142-9 may fuse, at step 706-9, the selected data from the device 108-9 and secondary devices 132-9. Fusion of data may describe multiple processes, and which process is performed may depend on the types of data being compared. For example, if both pieces or sets of data are measurements of the same parameter, such as glucose, then fusing the data may refer to aligning and combining data to ensure it is accurate and consistent. Aligning may be taking the mean or the median. The data may be enhanced, meaning an error is returned if both measurements are outside a predetermined normal range. For another example, fused data may be a correlation function of two sets of data, such as correlating real-time glucose measurements from the device 108-9 to sleep cycle readings from a secondary device 132-9 in order to track the effect of blood glucose on sleep quality. Fusing the data may refer to adjusting one piece or set of data based on the other. For example, pulse rate readings from a secondary device 132-9 may be used to adjust SP02 readings from the device 108-9. which are calibrated for a normal pulse rate. Fusing the data may refer to generating additional health parameters from the combination of the data. For example, blood glucose measurements from the device 108-9, blood cortisol levels from a secondary device 132-9, and heart rate measurements from another secondary device 132-9 can be fused to generate stress or activity level. Fusing data may refer to estimating a risk level based on the data sets. For example, blood glucose data from the device 108-9, heart rate data from a secondary device 132-9, and tissue oxygenation data from another secondary device 132-9 may all be factored into a risk score. The data fusion module 142-9 may use statistical algorithms, machine learning, and/or AI to perform the data fusion. The data fusion module 142-9 may send, at step 708-9, each piece or set of data and/or the fused data, if available, to the analysis module 144-9. The data fusion module 142-9 may return, at step 710-9, to step 700-9.

FIG. 79 illustrates an example operation of the analysis module 144-9. The analysis module 144-9 may poll, at step 800-9, for data from the data fusion module 142-9. This data may include fused data and unfused data. The analysis module 144-9 may add to, at step 802-9, the collected data by incorporating contextual information such as the patient's age, sex, weight, location, time of day, activity level, etc., if that information is available. For example, the system could combine heart rate data with information about the patient's location and exercise intensity to better understand the impact of their workout on their health. The analysis module 144-9 may incorporate additional contextual information such as medical history, genetic predispositions, or environmental factors to enhance the collected data. For example, the system could combine blood pressure data with the patient's age, family history of hypertension, and current stress levels to better understand their cardiovascular risk profile. This data may be supplied by the patient or their healthcare providers. This data may be stored in the admin database 138-9 or retrieved from another database external to the admin network 136-9, such as one containing the patient's medical history or a database on the patient's personal device, such as a smartphone. The analysis module 144-9 may also integrate data from wearable devices or mobile apps, providing real-time updates on the patient's lifestyle habits, such as diet and exercise routines, further enhancing the quality of the analysis. The analysis module 144-9 may input, at step 804-9, the new data, along with any contextual information, into one or more trained machine learning or AI algorithms. For example, the system could input patients' heart rate, exercise intensity, and location data to generate personalized insights about their cardiovascular health. The contextual data may influence the output. For example, a young obese patient with a fasting blood glucose level of 130 mg/dL may have outputs related to pre-diabetes, whereas the same blood glucose levels in an older, thinner patient may have outputs related to liver disease. The analysis module 144-9 may input the collected data and contextual information into multiple trained machine learning or AI algorithms, each specialized in analyzing specific aspects of the patient's health. For example, the system could input sleep pattern data and environmental factors into a dedicated sleep analysis algorithm while simultaneously inputting exercise data and genetic predispositions into a separate algorithm focused on physical fitness assessment. The analysis module 144-9 may also identify potential correlations between different health outcomes, enabling healthcare providers to simultaneously address the root cause of multiple health issues. The analysis module 144-9 may use external algorithms, such as GPT-4, through the use of the API 156-9. The analysis module 144-9 may use, at step 806-9, the trained predictive analytics algorithm to forecast future health outcomes based on the output from the algorithm or algorithms and/or the input data. For example, the system could predict the likelihood of a patient developing obesity or diabetes by analyzing trends in their physical activity, diet, and sleep patterns. This information could then be used to provide targeted interventions, such as personalized exercise plans or nutritional guidance, to help mitigate the risk of developing these health conditions. The analysis module 144-9 may utilize multiple predictive analytics algorithms to forecast future health outcomes for various aspects of the patient's well-being. Based on the outputs from multiple specialized algorithms, the analysis module 144-9 may predict the patient's risk of developing cardiovascular diseases, mental health disorders, or age-related conditions. For example, the output of the algorithm or algorithms in step 804-9 may indicate pre-diabetes and poor sleep quality, which may result in the predictive analytics algorithm's prediction that the patient is at high risk of early-onset Alzheimer's disease. These predictions could be combined to provide a holistic view of the patient's overall health status, enabling healthcare providers to design comprehensive, personalized intervention strategies addressing multiple health concerns. The analysis module 144-9 may also consider the patient's personal preferences, such as dietary restrictions or exercise preferences, to ensure that the generated insights are tailored to the individual and practical for implementation. The analysis module 144-9 may send, at step 808-9, the output data from the algorithm or algorithms in step 804-9 and/or the output of the predictive analytics algorithm in step 806-9 to the report module 148-9. The analysis module 144-9 may send the output data in a format that is easily understandable by the patient and healthcare providers, facilitating clear communication and collaboration for improving the patient's overall health. The data may also be sent to the simulated data module 150-9 and include the confidence interval of the final output and predictions. The analysis module 144-9 may return, at step 810-9, to step 800-9.

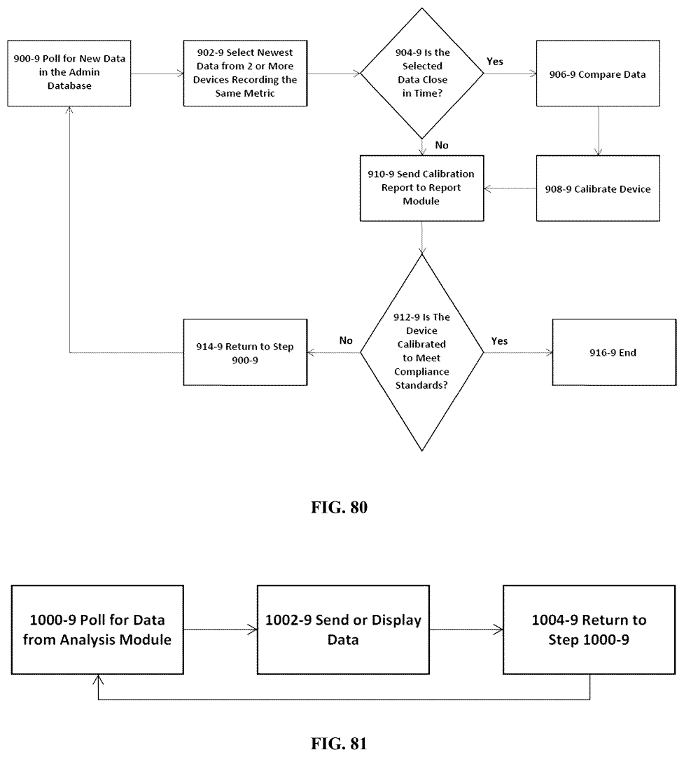

FIG. 80 illustrates an example operation of the calibration module 146-9. The calibration module 146-9 may poll, at step 900-9, for new data in the admin database 138-9. This new data may be data from the device 108-9 and/or from the secondary devices 132-9. The calibration module 146-9 may select, at step 902-9, the newest data from the device 108-9, the newest data from any 2 or more devices, including the device 108-9, and any secondary devices 132-9, which are recording the same metric. For example, the device 108-9 and two secondary devices 132-9 may all be recording blood glucose levels. The calibration module 146-9 may determine, at step 904-9, if the selected data is close in time. Close in time may mean each piece or set of data is timestamped within a few seconds of each other. For example, the newest data from the device 108-9 may be real-time, but the newest reading from a secondary device 132-9 may have been 10 minutes ago. This data would not be close in time if the threshold is a few seconds. The threshold may be varied based on the health parameter being measured. For example, if a secondary device 132-9 is recording sleep cycle activity, then recordings from 10 minutes ago may be relevant because of the gradual nature of sleep cycles. Still, if a secondary device 132-9 is recording blood glucose or electrolyte levels, then data from minutes ago may be highly inaccurate because these analytes can change rapidly. The time threshold for what data is considered close in time may be set by a user of the device, manufacturer of the device, and/or another module. If all of the selected data is not close in time, the calibration module 146-9 may skip to step 910-9 and report a failure to calibrate. The calibration module 146-9 may compare, at step 906-9, each device's data. This comparison helps to assess the accuracy of the device and identify any discrepancies between the sets of measurements, the calibration module 146-9 may utilize statistical techniques for comparing time-synchronized data obtained from each device. These techniques may include error analysis methods, such as calculating the Mean Absolute Error (MAE) or Root Mean Square Error (RMSE) between corresponding data points, as well as regression analysis methods, such as fitting a linear regression model to the data and evaluating the coefficient of determination (R2) to assess the degree of agreement between the two data sets. By employing these comparison methods, the system can identify discrepancies or trends in the data, enabling necessary adjustments to the algorithms to enhance the accuracy and reliability of the non-invasive RF sensor system. The calibration module 146-9 may wait until enough time-synchronized data points are obtained from the device 108-9 and the secondary devices 132-9 to perform a comprehensive comparison. These data points may be temporarily marked in the admin database 138-9 or stored in a separate database. The calibration module 146-9 may adjust, at step 908-9, the less accurate devices based on the recordings from the more accurate devices. For example, suppose the device 108-9, an external secondary device 132-9 and an internal secondary device 132-9 all recorded blood glucose levels. In that case, the device 108-9 and external secondary device 132-9 may be calibrated to closer match the internal secondary device 132-9 because the internal readings are presumed to be more accurate. Which devices are the most accurate may be indicated by an administrator of the network, found by comparing the specifications of each device from the manufacturer, or learned over time using a machine learning algorithm. The calibration module 146-9 may employ adaptive learning techniques to continuously refine the calibration process as more data is collected. This feature allows the system to improve its performance over time, adapting to the patient's unique physiology and changes in their health condition. The calibration module 146-9 may generate, at step 910-9, reports on the success or failure of the calibration process, as well as the degree of improvement achieved. These reports may be sent to the report module 148-9. These reports can be sent to an email address, displayed on a screen, forwarded to another module, or otherwise communicated to the user or healthcare provider. The calibration module 146-9 may determine, at step 912-9, if the device 108-9 is calibrated to meet compliance standards. For example, compliance standards may require the device 108-9 to give results within 0.5% of the results given by a secondary device 132-9. If the results from the device 108-9 are outside of this range within a set time period, such as over 24-hours, then the device 108-9 may not be compliant and may require further calibration or recalibration. Compliance standards may refer to legal standard, industry standard, standards set by the patient or their healthcare professionals, standards set by the manufacturer, etc. If multiple standards are used, the strictest standard may be used. If the device 108-9 does not meet compliance standards, the calibration module 146-9 may return, at step 914-9, to step 900-9. If the device 108-9 continuously fails to meet compliance standards, the calibration report generated at step 910-9 may indicate that the device 108-9 may be dysfunctional or that calibration alone cannot solve the non-compliance issue. If the device 108-9 meets the compliance standards, the calibration module 146-9 may end at step 916-9. The calibration module 146-9 may schedule a time to reinitiate itself, such as once a month or every 6 months, so that the device 108-9 may be recalibrated based on compliance standards. The calibration module 146-9 may reinitiate anytime a secondary device 132-9 with higher accuracy than the device 108-9 is connected to the system.

FIG. 81 illustrates an example of the report module 148-9. The report module 148-9 may poll, at step 1000-9, for data from the analysis module 144-9. If the calibration module 146-9 calibrated an instrument, the calibration report may also be obtained at this step. The report module 148-9 may send and/or display, at step 1002-9, the received data. If a display is connected to the admin network 136-9 or can be reached by other means, the report module 148-9 may display the data directly. If not, the report module may send data in real-time or periodically. Data may be sent to an email address, a database, a module, or any other destination. The data may be sent to and/or displayed at multiple locations. The report module 148-9 may return, at step 1004-9, to step 1000-9.

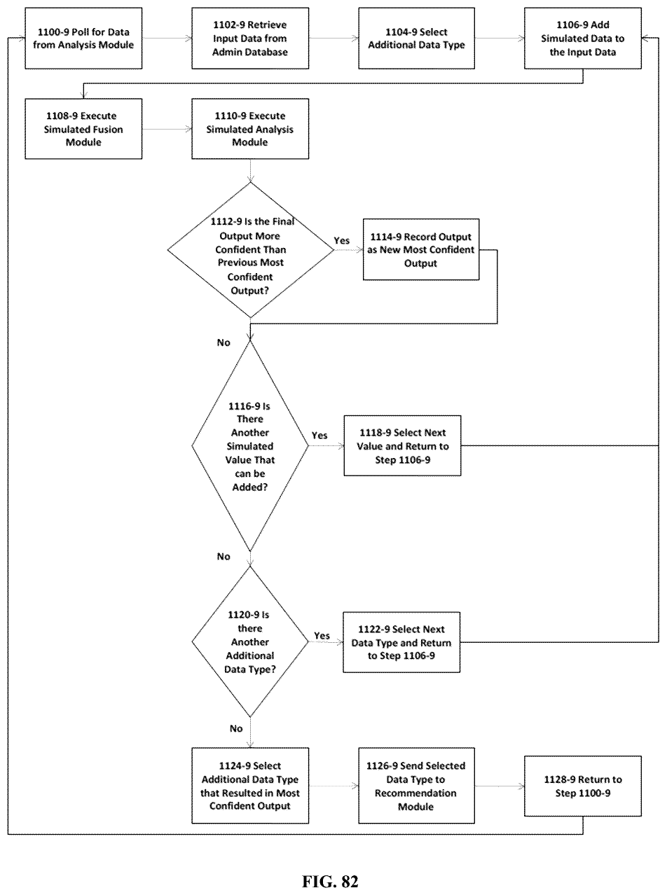

FIG. 82 illustrates an example operation of the simulated data module 150-9. The simulated data module 150-9 may poll, at step 1100-9, for data from the analysis module 144-9. The simulated data module 150-9 may retrieve, at step 1102-9, input data from the admin database 138-9 that was used to generate the output data from the analysis module 144-9. The simulated data module 150-9 may select, at step 1104-9, an additional data type. An additional data type may encompass various types of data that could provide supplementary information to enhance the analysis results. These additional data types may include but are not limited to, data from a blood test, diet information, or data from an Sp02 monitor. By incorporating these additional data types, the simulated data module 150-9 aims to improve the confidence level and accuracy of the output generated by the analysis module 144-9. The simulated data module 150-9 may add, at step 1106-9, simulated data to the input data. Simulated data may be a data point or set from the selected data type, which may be generated based on assumptions, statistical models, or historical data. The purpose of adding simulated data is to explore the potential impact of incorporating the selected data type on the analysis results and the confidence level of the final output. For example, if the selected data type is heart rate data, the simulated data may be a value of 70 bpm. By adding this simulated heart rate data to the input data, the simulated data module 150-9 can assess whether including heart rate information in the analysis would lead to a more confident or accurate output, ultimately guiding the recommendation module 152-9 to suggest the most appropriate measurements for improving the analysis. The simulated data module 150-9 may execute, at step 1108-9, the fusion module 142-9 using the combination of the original input data and the simulated data. This process aims to evaluate the potential impact of the simulated data on the overall analysis when integrated with the original input data. The fusion module 142-9, which is responsible for combining and processing multiple data sources, may be executed using the existing module or a duplicate module with the same functionality to ensure consistency in the data fusion process. By incorporating the simulated data with the original input data, the fusion module 142-9 can generate a new fused dataset that reflects the hypothetical addition of the selected data type, allowing the simulated data module 150-9 to assess the potential benefits of including this additional information in the analysis. The simulated data module 150-9 may execute, at step 1110-9, the analysis module 144-9 using the output of the fusion module 142-9 from step 1108-9. This execution aims to evaluate the impact of the simulated data on the overall results and confidence levels after being integrated with the original input data through the fusion module 142-9. The analysis module 144-9, which is responsible for interpreting and processing the fused data to generate meaningful insights and predictions, may be executed using the existing module or a duplicate module with the same functionality to maintain consistency in the analysis process. By applying the analysis module 144-9 to the new fused dataset that includes the simulated data, the simulated data module 150-9 can assess the potential improvements in the confidence levels or overall outcomes of the analysis, helping to determine whether the incorporation of the additional data type would be beneficial for the system's performance and decision-making. The simulated data module 150-9 may determine, at step 1112-9, if the final output with the added simulated data is more confident than the previous most confident output. The previous most confident output may refer to the original output generated without the inclusion of any simulated data or the output with the highest confidence interval achieved so far during the simulation process. This determination step aims to assess the potential improvement in the confidence level of the system's predictions or insights as a result of incorporating the additional data type. For example, suppose the original output without any simulated data has a confidence interval of 85%, and after adding heart rate data, the confidence interval increases to 88%. The simulated data module 150-9 would then compare this new output with the previous most confident output to decide whether the incorporation of heart rate data is beneficial. If the system further simulates the addition of another data type, such as blood oxygen level, and the confidence interval increases to 90%, the simulated data module 150-9 would update the previous most confident output with this new result. By continually evaluating the impact of incorporating different data types through simulation, the simulated data module 150-9 can identify the most beneficial additional data to improve confidence. If the final output is more confident than the previous most confident output, the simulated data module 150-9 may record, at step 1114-9, the output as the new most confident output. The simulated data module 150-9 may check, at step 1116-9, if there is another simulated value that can be added within the reasonable range for the selected data type. For example, if a simulated heart rate of 70 bpm was added to the input data previously, but 71 bpm has not been added, then there is another simulated value to be explored. The system can iterate through various values within a pre-defined range stepwise, such as every 1, 5, or 10 units, to evaluate their impact on the confidence level of the output. Not all possible values for a data type may be practical or relevant to use during the simulation process. For instance, when considering heart rate data, it may be impractical or unnecessary to use values below 30 bpm and above 200 bpm, as they might fall outside the typical range observed in the target population or may not provide meaningful insights. By focusing on a reasonable range of values for each data type, the simulated data module 150-9 can efficiently explore the potential impact of different data points on the system's output without expending excessive computational resources on unrealistic or irrelevant scenarios. If there is another simulated value, the simulated data module 150-9 may select, at step 1118-9, the next value and return to step 1106-9. If there is no other simulated value, the simulated data module 150-9 may check, at step 1120-9, if there is another additional data type. If there is another additional data type, the simulated data module 150-9 may select, at step 1122-9, the next data type and return to step 1106-9. If there are no other additional data types, the simulated data module 150-9 may select, at step 1124-9, the additional data type that resulted in the most confident output. Alternatively, the simulated data module 150-9 may rank the additional data types according to the confidence level they contribute to the system's output. This ranking may be based on the confidence intervals or other measures of certainty associated with the outputs generated using the simulated data of each additional data type. The simulated data module 150-9 may send, at step 1126-9, the selected data type to the recommendation module 152-9. The simulated data module 150-9 may return, at step 1128-9, to step 1100-9.

FIG. 83 illustrates an example operation of the recommendation module 152-9. The recommendation module 152-9 may poll, at step 1200-9, for data types from the simulated data module 150-9. The recommendation module 152-9 may identify, at step 1202-9, devices that record the data type received from the simulated data module 150-9 by searching a database, such as the admin database 138-9, for a list of compatible devices stored within the system. The identified devices may be associated with the specific data type, and the recommendation module may consider factors such as device availability, accuracy, and user preferences when identifying suitable devices. For example, if the data type received from the simulated data module 150-9 is blood oxygen level (Sp02), the recommendation module 152-9 may search for devices capable of measuring this metric, such as pulse oximeters. The recommendation module 152-9 may recommend, at step 1204-9, the identified device or devices capable of measuring the specified data type by presenting the recommendations to the user through various communication methods. The recommendation may be delivered through an on-screen display, such as a computer or mobile application user interface, where the user can view the recommended devices and select the preferred one. The recommendation may be sent via email, text message, or push notification, providing the user with details about the identified devices, their features, and potentially even purchasing options or nearby locations where the devices are available. The system may also provide additional context, such as an explanation of why the specific data type is important and how the recommended devices can help improve the confidence of the analysis results. The recommendation module 152-9 may return, at step 1206-9, to step 1200-9.

In one example implementation of Embodiment 9, a method for measuring one or more analytes using a real-time, non-invasive radio frequency (“RF”) analyte detection device can include: providing the real-time, non-invasive RF analyte detection device; providing at least one secondary device for recording health parameters; analyzing using a data analysis algorithm the data from the real-time, non-invasive RF analyte detection device and the at least one secondary device; determining that an additional source of data would improve the result of the analysis; and recommending the addition of the additional source of data to the analysis.

### Embodiment 10

FIG. 84 is a schematic illustration of a radio frequency health monitoring system. This system is configured to be attached to or in proximity to a body part 102-10. The body part 102-10 may be an arm 104-10. The body part 102-10 may be another body part 106-10 besides an arm, such as a leg, finger, chest, head, or any other body part from which useful medical parameters can be taken.

The system may comprise a device 108-10, which may be a wearable and portable device such as, but not limited to, a cell phone, a smartwatch, a tracker, a wearable monitor, a wristband, and a personal blood monitoring device.

The system may further comprise one or more transmit (“TX”) antennas 110-10. The one or more TX antennas 110-10 may be configured to transmit RF in the RF Activated Range from 500 MHZ to 300 GHZ. A predefined frequency may correspond to a range suitable for the human body in one embodiment. For example, the one or more TX antennas 110-10 would transmit radio frequency signals at a range of 120-126 GHz.

The system may further comprise one or more receive (“RX”) antennas 111-10. The one or more RX antennas 111-10 may be configured to receive the RF signals in response to the RF signal transmitted from the one or more TX antennas 110-10.

The system may further comprise an ADC converter 112-10, which may be configured to convert the received RF signals from an analog signal into a digital processor readable format.

The system may further comprise memory 114-10, which may be configured to store the transmitted RF signals by the one or more TX antennas 110-10 and store the received portion of the response or responded RF signals from the one or more RX antennas 111-10. Further, the memory 114-10 may also store the converted digital processor readable format by the ADC converter 112-10. The memory 114-10 may include suitable logic, circuitry, and/or interfaces that may be configured to store a machine code and/or a computer program with at least one code section executable by the processor 118-10. Examples of implementation of the memory 114-10 may include, but are not limited to, Random Access Memory (RAM), Read Only Memory (ROM), Hard Disk Drive (HDD), and/or a Secure Digital (SD) card.

The system may further comprise a standard waveform database 116-10, which may contain standard waveforms for known patterns. These may be raw or converted device readings from patients or persons with known conditions. For example, the standard waveform database 116-10 may include raw or converted device readings from a patient known to have diabetes or an average of multiple patients. This data can be compared to readings from a person with an unknown condition in order to determine if the waveforms taken from that person match any of the known standard waveforms.

The system may further comprise a processor 118-10, which may facilitate the operation of the device 108-10 according to the instructions stored in the memory 114-10. The processor 118-10 may include suitable logic, circuitry, interfaces, and/or code that may be configured to execute a set of instructions stored in the memory 114-10.

The system may further comprise a comms 120-10, which may be a communication element such as a Wi-Fi or RFC transmitter and receiver or physical connection that can send and receive data to other devices of the system and devices external to the system. The comms 120-10 may be configured to comply with regulatory acts such as HIPPA. For example, the comms 120-10 may include features such as encryption, access control, audit trails, secure transmission, credential verification, or other features which may protect the integrity and privacy of transmitted or received data.

The system may further comprise a battery 122-10, which may power hardware modules of the device 108-10. The device 108-10 may be configured with a charging port to recharge the battery 122-10. Charging of the battery 122-10 may be wired or wireless.

The system may further comprise a device base module 124-10, which may be configured to store instructions for executing the computer program from the converted digital processor readable format of the ADC converter 112-10. The device base module 124-10 may be configured to facilitate the operation of the processor 118-10, the memory 114-10, the one or more TX antennas 110-10, the one or more RX antennas 111-10, and the comms 120-10. Further, the device base module 124-10 may be configured to create polling of the RF Activated Range from 500 MHZ to 300 GHZ. It can be noted that the device base module 124-10 may be configured to filter the RF Activated Range from 500 MHZ to 300 GHZ received from one or more RX antennas 111-10.

The system may further comprise an input waveform module 126-10, which may extract a radio frequency waveform from memory. This may be the raw or converted recording of data from the one or more RX antennas 111-10 from a patient wearing the device. If the entire radio frequency is too long for effective matching, the input waveform module 126-10 may select a time interval within the data set. This input waveform may then be sent to the matching module 128-10.

The system may further comprise a matching module 128-10, which may match the input waveform and each of the standard waveforms in the standard waveform database 116-10 by performing a convolution and/or cross-correlation or other matching of the input waveform and the standard waveform. These convolutions and/or cross-correlations are then sent to the machine learning module.

The system may further comprise a machine learning module 130-10 which has been trained to identify health parameters based on the convolution and/or cross-correlations of the input and standard waveforms. The machine learning module 130-10 receives the convolutions and cross-correlations from the matching module 128-10 and outputs any health parameters identified.

The system may further comprise a device database 132-10, which may store the health parameters output by the machine learning module 130-10 and data collected from other devices such as the PAS device 140-10, NIRS device 144-10, and any additional sensors 148-10.

The system may further comprise a connection module 134-10, which may connect to the PAS device 140-10, NIRS device 144-10, and any additional sensors 148-10 and collect data which then may be stored in the device database 132-10.

The system may further comprise a data fusion module 136-10, which may fuse the real-time analyte data from the device 108-10 and collected data from the PAS device 140-10, NIRS device 144-10, and any additional sensors 148-10 stored in the device database 132-10. Fusing data may refer to the process of combining multiple sources of information or data sets into a single integrated output. This process involves aligning and combining data from different sources while ensuring it remains accurate and consistent.

The system may further comprise a report module 138-10, which may display a report of the fused data from the data fusion module 136-10. This report may be sent to an email address, displayed on a screen, sent to another module, or otherwise reported.

The system may further comprise a fusion rules database 139-10, which may contain rules for adjusting the input weights in the fusion process based on the data from the additional sensors. For example, when the additional sensors report that humidity is high, then the input data from the PAS device 140-10, which is sensitive to skin dampness, may have less impact on the fusion process results.

The system may further comprise a photoacoustic spectroscopy (PAS) device 140-10, which can be a wearable apparatus employing one or more photoacoustic spectroscopy sources for noninvasive analysis. The PAS device 140-10 can be situated on the patient's wrist or any other appropriate location. The device 140-10 may include a photoacoustic source and a detector configured to receive the photoacoustic signal generated from the patient's interstitial skin layer and/or blood. The PAS device 140-10 may further incorporate a processor and memory designed to process the photoacoustic signal and ascertain the concentration of analytes within the patient's blood. The photoacoustic source may be aimed at the patient's skin, and the detector can detect the photoacoustic signal produced through the interaction between the source and the patient's blood. The processor may process the signal to determine the concentration of various analytes in the patient's blood. The PAS device 140-10 may be calibrated to precisely measure analyte concentrations within the patient's blood. Calibration may be conducted using blood samples from the patient, which can be analyzed through conventional techniques. The PAS device 140-10 may be employed to monitor analyte concentrations continuously or periodically in the patient's blood. The PAS device 140-10 may furnish real-time feedback to the patient or healthcare professional, enabling them to take suitable actions based on the readings acquired from the device. The PAS device 140-10 may exhibit a compact and portable design for case of use and transportation. The PAS device 140-10 may also feature a user-friendly interface, displaying analyte concentrations intelligibly for patients and healthcare providers. The PAS device 140-10 may utilize single or multiple sources to generate photoacoustic signals from the patient's blood. The sources may possess different wavelengths to target distinct types of analytes, and the device may additionally comprise filters to decrease interference from background signals. To guarantee patient safety, the PAS device 140-10 may employ low-power sources and adhere to applicable safety standards and regulations. The PAS device 140-10 may also be designed to minimize discomfort or irritation to the patient's skin during use. The PAS device 140-10 may be applied in various medical contexts, including diabetes management, monitoring metabolic disorders, and detecting drug levels in the blood.

The system may further comprise the PAS comms 142-10, which may be a communication element such as a Wi-Fi or RFC transmitter and receiver or physical connection that can send and receive data to other devices of the system and devices external to the system. The PAS comms 142-10 may be configured to comply with regulatory acts such as HIPPA. For example, the PAS comms 142-10 may include features such as encryption, access control, audit trails, secure transmission, credential verification, or other features that may protect the data's integrity and privacy.

The system may further comprise a NIRS device 144-10, which may be a wearable device employing one or more near-infrared light sources for noninvasive spectroscopy. The NIRS device 144-10 may be worn on the patient's wrist or any other suitable location. The NIRS device 144-10 may consist of a near-infrared light source and a detector configured to detect the near-infrared signal from the patient's blood. The NIRS device 144-10 may also include a processor and a memory designed to analyze the near-infrared signal and determine the concentration of analytes in the patient's blood. The near-infrared light source may be directed onto the patient's skin, and the detector may detect the near-infrared signal generated by the interaction of the light with the patient's blood. The processor may analyze the signal to determine the concentration of various analytes in the patient's blood. The NIRS device 144-10 may be calibrated to provide accurate measurements of the concentration of analytes in the patient's blood. The calibration may be performed using blood samples obtained from the patient, which may be analyzed using conventional methods. The NIRS device 144-10 may be used to monitor the concentration of analytes in the patient's blood continuously or at regular intervals. The NIRS device 144-10 may provide real-time feedback to the patient or healthcare provider, enabling them to take appropriate action based on the readings obtained from the device. The NIRS device 144-10 may have a compact and portable design, allowing for easy use and transport. The NIRS device 144-10 may also feature a user-friendly interface, displaying the concentration of analytes in an intelligible format for both patients and healthcare providers. The NIRS device 144-10 may utilize a single or multiple light sources to generate near-infrared signals from the patient's blood. The light sources may have different wavelengths to target distinct types of analytes, and the NIRS device 144-10 may also include filters to decrease interference from background signals. To ensure patient safety, the NIRS device 144-10 may use low-power light sources and adhere to applicable safety standards and regulations. The NIRS device 144-10 may also be designed to minimize discomfort or irritation to the patient's skin during use. The NIRS device 144-10 may be applied in various medical contexts, including diabetes management, monitoring metabolic disorders, and detecting drug levels in the blood.

The system may further comprise the NIRS comms 146-10, which may be a communication element such as a Wi-Fi or RFC transmitter and receiver or physical connection that can send and receive data to other devices of the system and devices external to the system. The NIRS comms 146-10 may be configured to comply with regulatory acts such as HIPPA. For example, the NIRS comms 146-10 may include encryption, access control, audit trails, secure transmission, credential verification, or other features that may protect the integrity and privacy of transmitted or received data.

The system may further comprise one or more additional sensors 148-10, which may be employed to detect parameters that could introduce errors in the measurements obtained from the NIRS device 144-10 or PAS device 144-10. These additional sensors 148-10 may monitor factors such as patient movement, ambient temperature, electrical interference, and other environmental or physiological parameters that could affect the accuracy and reliability of the NIRS or PAS measurements. For example, a motion sensor or accelerometer may be incorporated into the system to monitor patient movement during the measurement process. Data from the motion sensor or accelerometer can be utilized to identify and correct motion artifacts, ensuring accurate and consistent readings from the NIRS device 144-10 or PAS device 140-10. For another example, a temperature sensor may be integrated into the system to measure the ambient temperature and compensate for its effects on the near-infrared or photoacoustic signals. The processor can use this information to correct the measurements and provide more accurate results. Other sensors, such as humidity, optical, or pressure sensors, may also be incorporated into the system to monitor and account for additional environmental or physiological factors that could affect the performance of the PAS device 140-10 or NIRS device 144-10. The processor can process and utilize data from these sensors to correct, adjust, or filter the measurements as needed. The additional sensors 148-10 may communicate with the PAS device 140-10 or NIRS device 144-10, sharing their data to enable real-time adjustments and corrections in the measurement process. The additional sensors 148-10 may be incorporated into the wearable device 108-10, or they may be separate components that communicate with the wearable device wirelessly or through a wired connection. The design of the additional sensors 148-10 and their integration into the system may be optimized for compactness, power efficiency, and ease of use.

The system may further comprise one or more additional sensor comms 150-10, which may be a communication element such as a Wi-Fi or RFC transmitter and receiver or physical connection that can send and receive data to other devices of the system and devices external to the system. The additional sensor comms 150-10 may be configured to comply with regulatory acts such as HIPPA. For example, the additional sensor comms 150-10 may include encryption, access control, audit trails, secure transmission, credential verification, or other features that may protect the integrity and privacy of transmitted or received data.

The system may further comprise any other spectroscopy devices such as a Raman laser device, elastic optical scattering spectroscopy device, mid-infrared spectroscopy device, ultraviolet-visible spectroscopy device, and/or fluorescent spectroscopy device. These devices may also connect via the connection module 134-10, have their data stored in the device database 132-10, and include that data in the data fusion process.

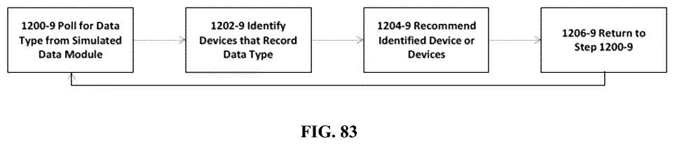

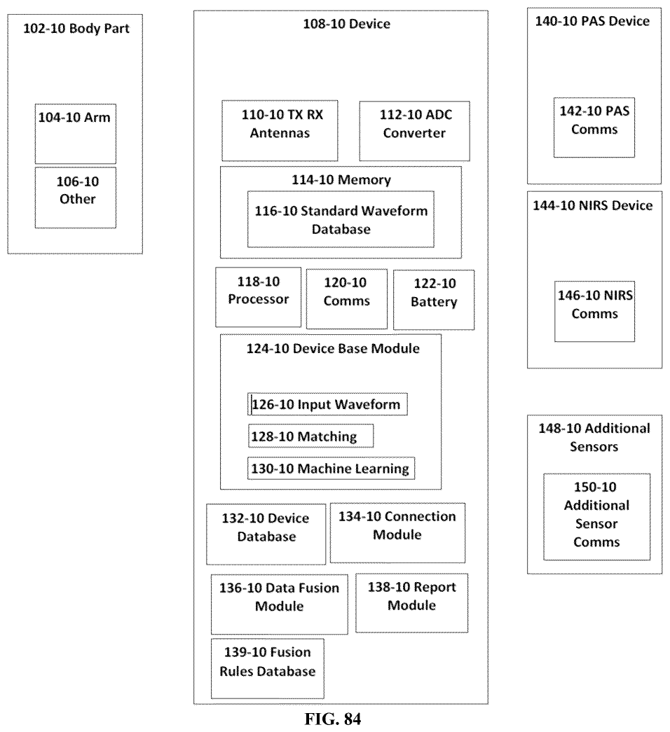

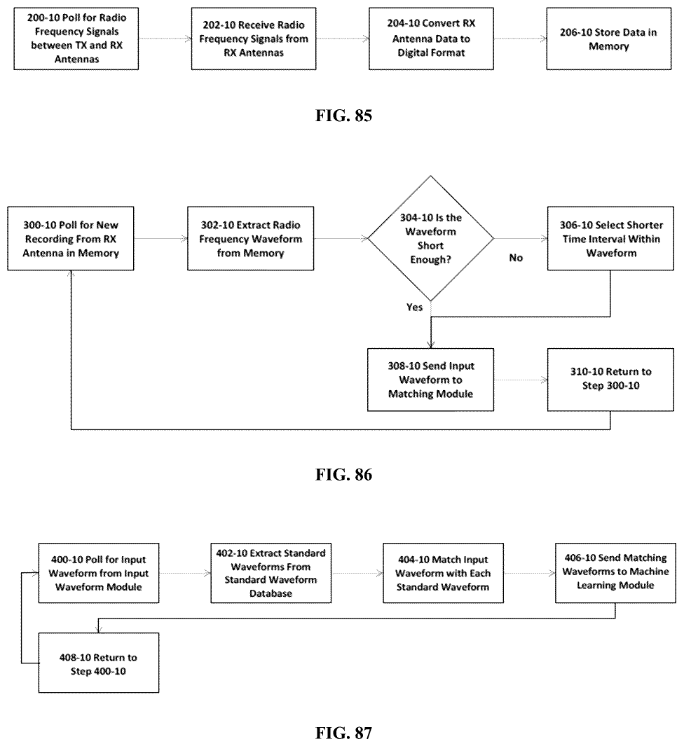

FIG. 85 illustrates an example operation of the device base module 124-10. The process may begin with the device base module 124-10 polling the Active Range RF signals between the one or more TX antennas 110-10 and the one or more RX antennas 111-10 at step 200-10. The device base module 124-10 may be configured to read and process instructions stored in the memory 114-10 using the processor 118-10. The one or more TX antennas 110-10 may be configured to transmit RF in the RF Activated Range from 500 MHZ to 300 GHZ. For example, the one or more TX antennas 110-10 may use RF signals at a range of 500 MHZ to 300 GHZ. The device base module 124-10 may receive the RF frequency signals from the one or more RX antennas 111-10 at step 202. For example, one or more RX antennas 111-10 receives a response or responded RF of frequency range 300-330 GHz from the patient's blood vessels. The device base module 124-10 may be configured to convert the received RF signals into a digital format using the ADC 112-10 at step 204. For example, the received RF of 300-330 GHz frequency range is converted into a 10-bit data signal. The device base module 124-10 may be configured to store converted digital format into the memory 114-10 at step 206-10.

FIG. 86 illustrates an example operation of the input waveform module 126-10. The process may begin with the input waveform module 126-10 polling, at step 300-10, for new recorded data from the one or more RX antennas 111-10 stored in memory 114-10. The input waveform module 126-10 may extract, at step 302-10, the recorded radio frequency waveform from memory. If there is more than one waveform recorded, the input waveform module 126-10 may select each waveform separately and loop through the following steps. The input waveform module 126-10 may determine, at step 304-10, if the waveform is small enough to be an input waveform for the matching module 128-10. This will depend on the computational requirements and/or restrictions of the matching module 128-10. If the waveform is short enough, the input waveform module 126-10 may skip to step 308-10. If the waveform is too long, the input waveform module 126-10 may select, at step 306-10, a shorter time interval within the entire recorded waveform. For example, if the waveform is 5 minutes long, then only a 30-second interval may be selected. The interval may be selected at random or by a selection process. The input waveform module 126-10 may send, at step 308-10, the input waveform to the matching module 128-10. The input waveform module 126-10 may return, at step 310-10, to step 300-10.

FIG. 87 illustrates an example operation of the matching module 128-10. The process may begin with the matching module 128-10 polling, at step 400-10, for an input waveform from the input waveform module 126-10. The matching module 128-10 may extract, at step 402-10, each standard waveform from the standard waveform database 116-10. The matching module 128-10 may match, at step 404-10, the input waveform with each standard waveform. Matching may be determining which standard waveforms the input waveform is similar to. Matching may involve convolution and/or cross-correlation of the waveforms or any other matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function whose values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-10. The matching module 128-10 may send, at step 406-10, the matching waveforms to the machine learning module 130-10. Matching waveforms may refer to the standard waveforms that were similar to the input waveform, the waveforms that were generated via convolution and/or cross-correlation, or both. The matching module 128-10 may return, at step 408-10, to step 400-10.

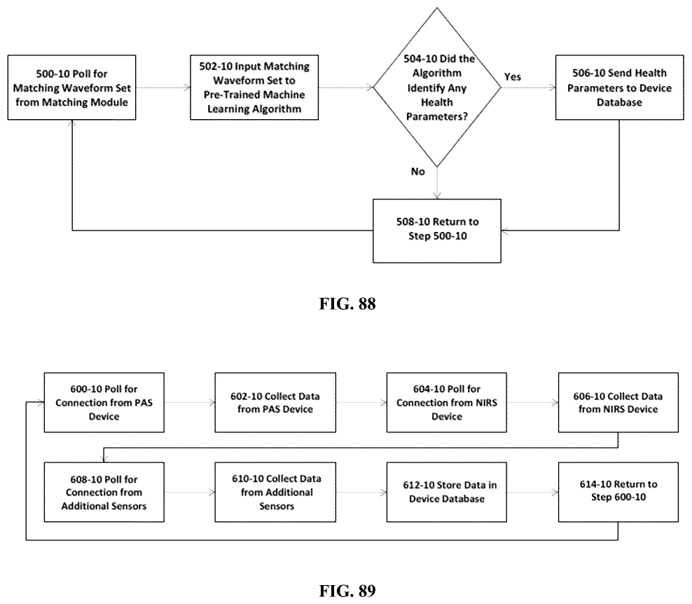

FIG. 88 illustrates an example operation of the machine learning module 130-10. The process may begin with the machine learning module 130-10 polling, at step 500-10, for a set of matching waveforms from the matching module 128-10. Matching waveforms may be a set of standard waveforms that are similar to the input waveform or statistical combinations of the input waveform and standard waveforms, such as convolutions or cross-correlations. The machine learning module 130-10 may input, at step 502-10, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During the training process, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-10 may determine, at step 504-10, if the algorithm identified any health parameters. Identification may require a certain interval of confidence. For example, if the machine learning algorithm determines that it is more than 70% likely that a health parameter is correct, then that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130-10 may skip to step 508-10. If any health parameters were identified, the machine learning module 130-10 may send, at step 506-10, the health parameters to the device database 132-10. The machine learning module 130-10 may return, at step 508-10, to step 500-10.

FIG. 89 illustrates an example operation of the connection module 134-10. The process may begin with the connection module 134-10 polling, at step 600-10, for a connection from the PAS device 140-10. If no connection is detected, the connection module 134-10 may search for a PAS device that is attempting to connect or may skip to step 604-10. The connection module 134-10 may collect, at step 602-10, data from the PAS device. This may be real-time data or periodically updated data. The connection module 134-10 may poll, at step 604-10, for a connection from the NIRS device 144-10. If no connection is detected, the connection module 134-10 may search for a NIRS device 144-10 that is attempting to connect or may skip to step 608-10. The connection module 134-10 may begin at this step if the PAS Device 140-10 is unavailable or unnecessary. The connection module 134-10 may collect, at step 606-10, data from the NIRS device 144-10. This may be real-time data or periodically updated data. The connection module 134-10 may poll, at step 608-10, for a connection from one or more additional sensors 148-10. If no connection is detected, the connection module 134-10 may search for an additional sensor 148-10 that is attempting to connect or may skip to step 612-10. The connection module 134-10 may collect, at step 610-10, data from the additional sensors 148-10. This may be real-time data or periodically updated data. The connection module 134-10 may store, at step 612-10, the data from the PAS device 140-10, NIRS device 144-10, and/or additional sensors 148-10 in the device database 132-10. The connection module 134-10 may add contextual data such as the ID of the PAS device 140-10, the ID of the NIRS device 144-10, the type of additional sensor 148-10, the analyte measured by the NIRS device 144-10, time data was retrieved, etc., to the received data before storage in the device database 132-10. The connection module 134-10 may return, at step 614-10, to step 600-10.

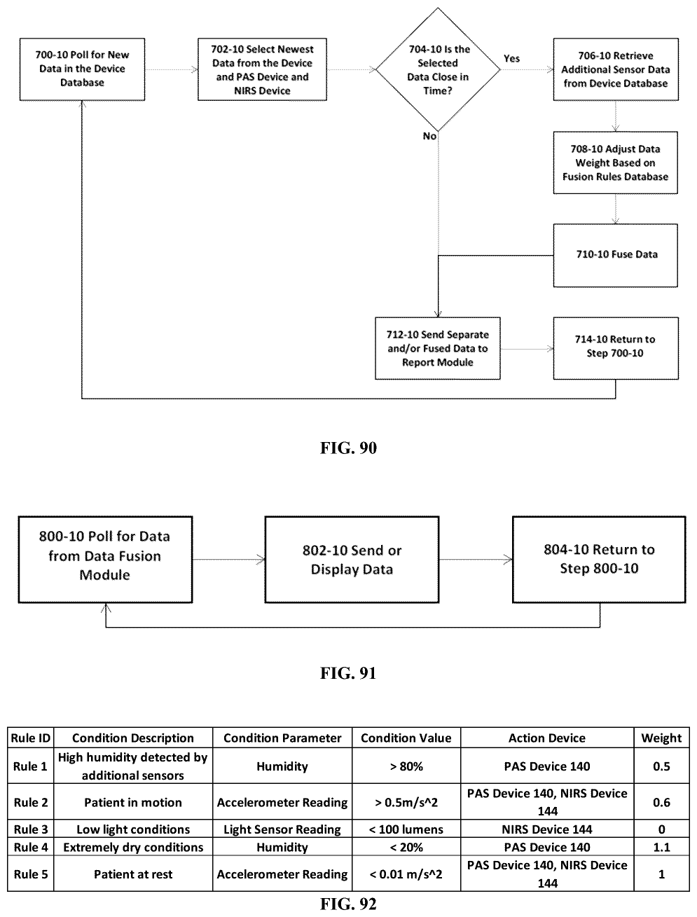

FIG. 90 illustrates an example operation of the data fusion module 136-10. The process may begin with the data fusion module 136-10 polling, at step 700-10, for new data in the device database 132-10. This new data may be from device 108-10, the PAS device 140-10, or the NIRS device 144-10. The data fusion module 136-10 may select, at step 702-10, the newest data from the device 108-10, the newest data from the PAS device 140-10, and the newest data from the NIRS device 144-10. This may be a single data point or a set of data. The data fusion module 136-10 may determine, at step 704-10, if the selected data is close in time. Close in time may mean each piece or set of data is timestamped within a few seconds of each other. For example, the newest data from the device 108-10 may be real-time, but the newest reading from the NIRS device 144-10 may have been 10 minutes ago. This data would not be close in time if the threshold is a few seconds. The threshold may be varied based on the health parameter being measured. For example, if the PAS device 140-10 is recording cholesterol, then recordings from 10 minutes ago may be relevant because cholesterol level does not change rapidly, but if the NIRS device 144-10 records blood glucose or electrolyte levels, then data from minutes ago may be highly inaccurate. The time threshold for what data is considered close in time may be set by a device user, manufacturer of the device, and/or another module. If the selected data is not close in time, the data fusion module may skip to step 708-10. The data fusion module 136-10 may retrieve, at step 706-10, data from the additional sensors 148-10 stored in the device database 132-10. The data fusion module 136-10 may only retrieve additional sensor 148-10 data that is close enough in time to the other collected data to be relevant. For example, if an additional sensor 148-10 is recording humidity and the last recording was 1 minute ago, then this recording is likely still relevant because humidity doesn't change much in a minute. However, if the additional sensor 148-10 is recording electrical interference, then a recording from 1 minute ago may be irrelevant because a new electrical device may have been turned on or off at the last minute. The data fusion module 136-10 may adjust, at step 708-10, the weight of each source of spectroscopy data based on rules found in the fusion rules database 139-10. The additional sensor 148-10 data may be used to exclude or adjust the effect of data from the device 108-10, PAS device 140-10, and/or NIRS device 144-10 on the fused data. For example, suppose the data from the additional sensors 148-10 indicates that humidity is above 80%. In that case, the readings from the PAS device 140-10 may be ignored, or have diminished contribution, because PAS is very sensitive to humidity due to the high absorption of infrared excitation light to the skin secretion products or water. For another example, if the additional sensor 148-10 is an accelerometer that indicates the patient is moving more than 0.5 m/s\hat{ }2, then the data from both the PAS device 140-10 and NIRS device 144-10 may have a diminished effect on the results of data fusion because both devices are affected by motion. If all but one source of data is ignored, then fusion may be skipped entirely since there is only one reliable source of data. The data fusion module 136-10 may fuse, at step 710-10, the selected data from the device 108-10, PAS device 140-10, and/or NIRS device 144-10. The fusion of data may describe multiple processes, and which process is performed may depend on the types of data being compared. For example, if both pieces or data sets are measurements of the same parameter, such as glucose, then fusing the data may refer to aligning and combining data to ensure it is accurate and consistent. Aligning may include the mean or median with weights from step 708-10. The data may be enhanced, meaning an error is returned if both measurements are outside a predetermined normal range. For another example, fused data may be a correlation function of two data sets, such as correlating real-time glucose measurements from device 108-10 to insulin from PAS device 140-10 to track blood insulin's effect on blood glucose. Fusing the data may refer to adjusting one piece or set of data based on the other. For example, glucose readings from the PAS device 140-10 may be used to adjust SP02 readings from the NIRS device 144-10, which are calibrated for a normal glucose level. Fusing the data may refer to generating additional health parameters from the combination of the data. For example, blood glucose measurements from the device 108-10, blood cortisol levels from the NIRS device 144-10, and endorphin levels from the PAS device 140-10 can be fused to generate stress or activity level. Fusing data may refer to estimating a risk level based on the data sets. For example, blood glucose data from the device 108-10, red blood cell count from the PAS device 140-10, and tissue oxygenation data from the NIRS device 144-10 may all be factored into a risk score. The data fusion module 136-10 may use statistical algorithms, machine learning, and/or AI to perform the data fusion. The data fusion module 136-10 may send, at step 712-10, each piece or set of data and/or the fused data, if available, to the report module 138-10. The data fusion module 136-10 may return, at step 714-10, to step 700-10.

FIG. 91 illustrates an example operation of the report module 138-10. The process may begin with the report module 138-10 polling, at step 800-10, for data from the data fusion module 136-10. The report module 138-10 may send and/or display, at step 802-10, the received data. If a display is connected to the device 108-10 or can be reached by other means, the report module 138-10 may display the data directly. If not, the report module may send data in real-time or periodically. Data may be sent to an email address, a database, a module, or any other destination. The data may be sent to and/or displayed at multiple locations. The report may include the fused data, the data from each device used to create the fused data, such as the device 108-10, PAS device 140-10, and NIRS device 144-10, and data from the additional sensors 148-10. This data may be overlayed, displayed side-by-side, or placed on a table. The report module 138-10 may return, at step 804-10, to step 800-10.

FIG. 92 illustrates an example of the fusion rules database 139-10. The fusion rules database 139-10 may contain the following columns. The “Condition Description” column describes the condition that needs to be met for the rule to apply. The “Condition Parameter” column represents the parameter that will be checked. The “Condition Value” column represents the threshold or condition the parameter should meet for applying the rule. The “Action Device” column shows which device's data contribution will be adjusted if the rule applies. If the rule applies, the “Weight” column shows the weight that will be given (i.e., whether the device's data contribution will be diminished, ignored, or enhanced). The fusion rules database 139-10 might contain more complex rules and conditions, depending on the system's specific requirements.

In one example implementation of Embodiment 10, a method for measuring one or more analytes using a real-time, noninvasive radio frequency (“RF”) analyte detection device can include the following: providing the real-time, noninvasive RF analyte detection device; providing at least one spectroscopy device selected from the group consisting of a photoacoustic spectroscopy device, a near-infrared spectroscopy device, and any other spectroscopy device; providing at least one additional sensor; providing a connection module which connects the real-time, noninvasive RF analyte detection device, the at least one spectroscopy device, and the at least one additional sensor; storing a first set of data of the one or more analytes obtained from the real-time, noninvasive RF analyte detection device; storing a second set of data of the one or more analytes obtained from the at least one spectroscopy device; storing a third set of data obtained from the at least one additional sensor; executing a fusion module to form fused data from at least two sets of data selected from the group consisting of the first set of data, the second set of data, and the third set of data; reporting the fused data results.

### Embodiment 11

FIG. 93 illustrates a system fusing data from a non-invasive RF device with MRI data. This system may be comprised of a user with a body part 102-11, such as any part of a human, including a limb or extremity.

Further, embodiments may include the user having an arm 104-11, such as either of the two upper limbs of the human body from the shoulder to the hand.

Further, embodiments may include other extremities 106-11 or other parts of the body, such as a limb, such as a leg or a torso, or another body part 102-11 besides an arm 104-11, such as a leg, finger, chest, head, or any other body part 102-11 from which useful medical parameters can be taken.

Further, embodiments may include a device 108-11, such as a non-invasive RF device 108-11, which may contain a plurality of TX antennas 110-11, a plurality of RX antennas 112-11, an ADC converter 114-11, memory 116-11, processor 120-11, communication interface 122-11, battery 124-11, user interface 128-11, and a base module 134-11 containing a plurality of sub-modules. The device 108-11 may be a handheld or maneuverable device 108-11 to allow medical professionals to reposition the device 108-11 when needed. In some embodiments, the non-invasive RF device 108-11 may include RF sensors which may be used to measure inflammation in the body. RF sensors may be used for cancer detection, particularly in the context of detecting circulating tumor cells (CTCs) or other biomarkers associated with cancer.

Further, embodiments may include a plurality of TX antennas 110-11 which may be integrated into the circuitry arrangement. The one or more TX antennas 110-11 may be configured to transmit the Activated RF range signals at a predefined frequency. In one embodiment, the predefined frequency may correspond to a range suitable for the human body. For example, the one or more TX antennas 110-11 transmit Activated RF range signals at a range of 120-126 GHz.

Further, embodiments may include a plurality of RX antennas 112-11 which may be integrated into the circuitry arrangement. The one or more RX antennas 112-11 may be configured to receive the responded portion of the Activated RF range signals. In one embodiment, the Activated RF range signals may be transmitted to the user's skin, and electromagnetic energy may be responded from many parts such as fibrous tissue, muscle, tendons, bones, and the skin. It can be noted that effective monitoring of the analyte levels is facilitated by an electrical response of blood against the transmitted Activated RF range signals. Further, the electromagnetic energy responded from the blood may be received by the one or more RX antennas 112-11.

Further, embodiments may include an ADC converter 114-11 which may be coupled to the one or more TX antennas 110-11. The one or more RX antennas 112-11 may be configured to receive the responded Activated RF range signals. The ADC 114-11 may be configured to convert the Activated RF range signals from an analog signal into a digital processor readable format.

Further, embodiments may include a memory 116-11 may be configured to store the transmitted Activated RF range signals by the one or more TX antennas 110-11 and receive a responded portion of the transmitted Activated RF range signals from the one or more RX antennas 112-11. Further, the memory 116-11 may also store the converted digital processor readable format by the ADC 114-11. In one embodiment, the memory 116-11 may include suitable logic, circuitry, and/or interfaces that may be configured to store a machine code and/or a computer program with at least one code section executable by the processor 120-11. Examples of implementation of the memory 116-11 may include, but are not limited to, Random Access Memory (RAM), Read Only Memory (ROM), Hard Disk Drive (HDD), and/or a Secure Digital (SD) card.

Further, embodiments may include a standard waveform database 118-11, which may contain standard waveforms for known patterns. These may be raw or converted device 108-11 readings from patients or persons with known conditions. For example, the standard waveform database 118-11 may include raw or converted device 108-11 readings from patients known to have inflammation due to cancerous cells or tumors or an average of multiple patients. This data can be compared to readings from a person with an unknown condition in order to determine if the waveforms taken from that person match any of the known standard waveforms. The standard waveform database 118-11 may contain the transmitted RF signals by the TX antennas 110-11, the received RF signals by the RX antennas 112-11, the analyte associated with signals, and the measurement of the analyte, such as 5 CTCs, or circulating tumor cells, per 7.5 ml. The standard waveform database 118-11 may contain a plurality of historical data entries of historical patients with the RF signals, both transmitted and received, the analyte, and the analyte levels. For example, the analytes may be CTCs which would indicate cancerous cells in the bloodstream, C-reactive Protein, interleukin-6, or tumor necrosis factor-alpha, which would be an indicator of inflammation that may be caused by cancer.

Further, embodiments may include a processor 120-11, also known as a central processing unit (CPU), which may facilitate the operation of the device 108-11 according to the instructions stored in the memory 116-11. The processor 120-11 may include suitable logic, circuitry, interfaces, and/or code that may be configured to execute a set of instructions stored in the memory 116-11. The processor 120-11 may be a hardware component that performs arithmetic, logic, and control operations on data. The processor 120-11 may comprise an arithmetic logic unit, control unit, memory subsystems, and other subsystems. The processor 120-11 may perform arithmetic and logical operations on data. The processor 120-11 may include components for addition, subtraction, multiplication, and division and logical operations such as AND, OR, and NOT. The processor 120-11 may be responsible for fetching instructions from memory 116-11, decoding them, and executing them. The processor 120-11 may manage the flow of data between different components of the system as a whole, ensuring that operations are performed in the correct order, and that data is transferred efficiently. The processor 120-11 may provide fast access to frequently used data and instructions. The processor 120-11 may include components such as caches, registers, and pipelines, which are designed to minimize the time required to access and manipulate data. The processor 120-11 may include various other components and subsystems, such as instruction set architecture (ISA), which may define the set of instructions that the processor 120-11 can execute. The processor 120-11 may specify the format of instructions and data, the addressing modes used to access memory 116-11 and I/O devices, and the interrupt and exception handling mechanisms used to manage errors and other events. The processor 120-11 may include advanced instruction execution capabilities, support for virtualization and parallel processing, and power management mechanisms that reduce energy consumption and heat dissipation.

Further, embodiments may include a communication interface 122-11, which may be a hardware or software component that enables communication between two or more electronic devices or systems. The communication interface 122-11 may include a set of protocols, rules, and standards that define how information is transmitted and received between the devices. The communication interfaces 122-11 may be a physical connector, wireless network, or software application and may include components such as drivers, software libraries, and firmware that may be used to control and manage the communication process. In some embodiments, the communication interface 122-11 may be compatible with USB, Bluetooth, or Wi-Fi. The communication interface 122-11 may communicate with a network. Examples of networks may include but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN).

Further, embodiments may include a battery 124-11, which may power hardware modules of the device 108-11. The device 108-11 may be configured with a charging port to recharge the battery 124-11. Charging of the battery 124-11 may be wired or wireless. The battery 124-11 may be a device that is used to store and release electrical energy. The battery 124-11 may consist of one or more electrochemical cells, which convert chemical energy into electrical energy. The battery 124-11 may contain one or more electrodes, which may be made of a metal or metal oxide, and an electrolyte, which is a substance that conducts electricity. When charging the battery 124-11, a chemical reaction occurs within the electrochemical cell, which causes electrons to flow from one electrode to the other, producing a flow of electrical energy. A plurality of electrochemical cells within the battery 124-11 may be configured in a plurality of ways.

Further, embodiments may include a screen 126-11 which may be a hardware component that provides visual output for a computer system, also known as a monitor or display. The screen 126-11 may be comprised of a panel, controller, and other subsystems. The screen 126-11 may be the physical component that emits light and creates the image displayed on the screen. The screen 126-11 may consist of a thin film transistor (TFT) layer and a liquid crystal layer, which work together to control the flow of light through the panel and create an image. The screen 126-11 may include components for decoding video signals, and scaling images to the appropriate size and resolution to create the final image. The screen 126-11 may include backlighting systems which provide uniform illumination across the panel and enhance the brightness and contrast of the displayed image. The screen 126-11 may include touch-sensitive screens, which enable users to interact with the system using gestures and touch inputs. Screens 126-11 may also include advanced color management systems, which provide accurate color reproduction and enable users to calibrate the screen 126-11 to match their preferences and requirements.

Further, embodiments may include a user interface 128-11, which may be a software component that enables users to interact with a computer system and access its functionalities. The user interface 128-11 may be comprised of an input component, an output component, and other functions. The user interface 128-11 may provide users with a means of inputting data and commands into the system. The user interface 128-11 may include a keyboard, mouse, touchscreen, or other input devices and may be responsible for translating user inputs into machine-readable formats that can be processed by the system. The user interface 128-11 may provide users with feedback on their actions and the system's status. The user interface 128-11 may include a screen 126-11, speakers, or other output devices and may be responsible for rendering visual, auditory, or other types of feedback to the user. The user interface 128-11 may include navigation systems that enable users to move between different system areas and access different functionalities. The user interface 128-11 may include menus, toolbars, or other elements that provide visual cues and options for the user. The user interface 128-11 may include support for multiple languages, accessibility options for users with disabilities, and customization options that enable users to personalize the interface to their preferences.

Further, embodiments may include a controller 130-11, which may be responsible for controlling and managing the operation of the device 108-11. The controller 130-11 may include a set of algorithms, rules, and logic circuits that are used to process input signals and generate output signals to control the behavior of the device 108-11. In some embodiments, the controller 130-11 may be a microcontroller, programmable logic controller (PLC), or digital signal processor (DSP) and may be implemented as software components running on general-purpose computers or embedded systems.

Further, embodiments may include a location sensor 132-11, such as an accelerometer, which may be used to measure acceleration or changes in motion. The location sensor 132-11 may consist of a mass suspended by springs, which can move in response to changes in acceleration. The motion of the mass is detected by one or more sensors, which convert the motion into an electrical signal that can be processed and analyzed. The sensors used in the location sensor 132-11 may include piezoelectric, capacitive, optical, etc. The location sensor 132-11 may be used to determine the device's 108-11 location on the patient's body. For example, the location sensor 132-11 is integrated into the device 108-11 in a position that allows it to measure the desired motion or orientation. Then before using the location sensor 132-11 to measure motion or orientation, the location sensor 132-11 is calibrated to ensure accurate readings. The calibration may involve setting the zero point, such as the point where there is no acceleration and determine the sensitivity of the device 108-11. Then the location sensor 132-11 produces data in the form of acceleration values along the three axes (X, Y, and Z). Integrating these values over time determines the velocity and position of the device 108-11. Filtering and signal processing techniques may be used to clean up the data and extract the desired information. Once the data has been processed, it can be analyzed to determine the device's 108-11 position on the body. In some embodiments, the location sensor 132-11 may combine accelerometer data with other sensors, such as gyroscopes and magnetometers, to better measure the device's 108-11 positions and orientation.

Further, embodiments may include base module 134-11, which begins with the medical professional positioning the device 108-11 on the patient. The base module 134-11 initiates the scan module 136-11. The base module 134-11 determines if the medical professional repositioned the device 108-11. If it is determined that the medical professional repositioned the device 108-11, the base module 134-11 returns to initiating the scan module 136-11. If it is determined that the medical professional did not reposition the device 108-11, the base module 134-11 connects to the MRI imaging device 150-11. The base module 134-11 continuously polls to receive the MRI image data from the MRI imaging device 150-11. The base module 134-11 receives the MRI image data from the MRI imaging device 150-11. The base module 134-11 stores the MRI image data in the patient database 146-11. The base module 134-11 initiates the fusion module 140-11. The base module 134-11 initiates the information module 142-11.

Further, embodiments may include a scan module 136-11, which begins by being initiated by the base module 134-11. The scan module 136-11 sends the RF transmit signal to the TX antennas 110-11. The scan module 136-11 stores the RF transmit signal to memory 116-11. The scan module 136-11 receives the RF signal from the RX antennas 112-11. The scan module 136-11 converts to digital using the ADC converter 114-11. The scan module 136-11 stores the RX converted signal data in memory 116-11. The scan module 136-11 correlates the RF signals with ground truth data to determine the analyte levels. The scan module 136-11 stores the analyte levels in the patient database 146-11. The scan module 136-11 initiates the location module 138-11. The scan module 136-11 compares analyte levels to the threshold database 144-11. The scan module 136-11 determines if the analyte levels are over the thresholds stored in the threshold database 144-11. If it is determined that the analyte levels are over the thresholds stored in the threshold database 144-11, the scan module 136-11 connects to the MRI module 158-11. The scan module 136-11 extracts the analyte levels and the location data from the patient database 146-11. The scan module 136-11 sends the analyte levels and the location data to the MRI module 158-11. If it is determined that the analyte levels are not over the thresholds stored in threshold database 144-11 or after the data is sent to the MRI module 158-11, the scan module 136-11 returns to the base module 134-11.

Further, embodiments may include a location module 138-11, which begins by being initiated by the scan module 136-11. The location module 138-11 collects the location data from the location sensor 132-11. The location module 138-11 determines the location of the device 108-11. The location module 138-11 stores the location data in the patient database 146-11. The location module 138-11 returns to the scan module 136-11.

Further, embodiments may include a fusion module 140-11, which begins by being initiated by the base module 134-11. The fusion module 140-11 extracts the analyte data and MRI image data from the patient database 146-11. The fusion module 140-11 performs the fusion model on the data. The fusion module 140-11 stores the results from the fusion model in the patient database 146-11. The fusion module 140-11 returns to the base module 134-11.

Further, embodiments may include an information module 142-11 which begins with the information module 142-11 being initiated by the base module 134-11. The information module 142-11 extracts the fusion model results from patient database 146-11. The information module 142-11 displays the fusion model results data on the user interface 128-11. The information module 142-11 returns to the base module 134-11.

Further, embodiments may include a threshold database 144-11, which may contain thresholds for various analyte levels compared to the analyte levels collected by the device 108-11 during the scan module 136-11 to determine if an MRI image should be taken of the area. The threshold database 144-11 may contain the type of analyte and a threshold that indicates potential inflammation in the area. For example, the levels of certain analytes can be elevated or reduced in response to inflammation or other pathological processes in the body. C-reactive protein (CRP) is an analyte commonly measured in the blood to detect inflammation as its levels increase in response to inflammation. Circulating tumor cells, or CTCs, are cancer cells from a tumor traveling in the bloodstream. CTCs provide genetic information about cancer, which can provide the tumor's location, inform medical professionals about the best treatments, etc. CTCs and their clusters disseminate to distant bodily sites through the bloodstream as part of the metastatic process. Their occurrence is rare, often<100 CTCs or <10 clusters per 10×106 leukocytes and 5×109 erythrocytes in 1 ml whole blood. Nevertheless, their occurrence far exceeds the number of actual metastatic lesions in patients, supporting the notion that very few CTCs overcome the harsh vascular environment and successfully metastasize onto a secondary site. Other analytes, such as cytokines or chemokines, can also be indicators of inflammation or other immune responses. The analyte levels or concentrations may provide important information about the presence and severity of inflammation or other pathological processes in the body, such as cancer. C-reactive protein (CRP), circulating cancer cells, cytokines, and chemokines may be used to detect cancer-related inflammation in the blood. For example, CRP is an acute-phase protein produced in response to inflammation, including cancer-related inflammation. Elevated levels of CRP in the blood are often seen in patients with various types of cancer, including lung, breast, colorectal, and pancreatic cancer. Levels greater than 10 mg/L are generally considered to indicate the presence of acute inflammation. Also, cytokines and chemokines are signaling molecules that play important roles in immune responses and inflammation. Some cytokines and chemokines are produced by cancer cells or immune cells in response to cancer, and their levels in the blood can be used to detect cancer-related inflammation. Interleukin-6 (IL-6) is a cytokine that is often elevated in patients with cancer, particularly those with advanced disease, and its levels can be used to monitor treatment response and predict prognosis. A cytokine, such as interleukin, may have levels greater than 100 μg/mL, indicating patients with severe infections or other inflammatory conditions. Another cytokine, such as tumor necrosis factor-alpha, may have levels greater than 100 μg/mL, indicating patients with sepsis, autoimmune diseases, or other inflammatory conditions. Lastly, the detection of CTCs in the bloodstream indicates the presence of tumor cells in the bloodstream, which can indicate active cancer in the body. The presence of 5 CTCs per 7.5 millimeters of blood would indicate an increased risk of cancer and a poorer prognosis.

Further, embodiments may include a patient database 146-11, which may contain the data collected from the device 108-11, MRI imaging device 150-11, and the results of the fusion model. The patient database 146-11 may contain patient ID, date, time, a first analyte, an analyte “N” indicating an infinite number of potential analyte readings, the location data, the MRI image data, and the fusion model results. The patient database 146-11 may contain the analyte data, such as the analyte levels or readings captured from the device 108-11, stored as a data file which may include information such as the type of analyte, the analyte readings, etc. The patient database 146-11 may contain the location data such as the device 108-11 location at the time of the readings, which may be a region of the patient's body, a specific body part 102-11, or an exact location of where the device 108-11 was located on the patient's body. The MRI data may be 3D images that can be viewed from different angles. The fusion model results may be a data file that contains the analyte readings data, and the MRI image data overlayed on one another to provide a healthcare provider with on graphical representation of the data, such as the MRI image data with the location of the device 108-11 and the analyte readings in the section of the device 108-11.

Further, embodiments may include a cloud 148-11, which may be a network of remote servers that provide on-demand computing resources and services over the Internet. The cloud 148-11 may consist of a collection of servers, storage devices, and networking equipment. Users may access the cloud 148-11 through various devices, such as computers, smartphones, and tablets, using internet connectivity. In some embodiments, the architecture of the cloud 148-11 may be based on a distributed computing model, with multiple servers working together to provide services to users.

Further, embodiments may include an MRI imaging device 150-11, which may be magnetic resonance imaging, or MRI, device such as a medical imaging system that uses a strong magnetic field, radiofrequency pulses, and gradient fields to produce detailed images of the body's internal structures. The MRI imaging device 150-11 may consist of several components, including a main magnet, gradient coils, RF coils, a patient table, a computer, software, a cooling system, a power supply, and safety systems. The MRI imaging device 150-11 generates a strong, uniform magnetic field that aligns the protons in the body's tissues. The MRI imaging device 150-11 produces a varying magnetic field that is used to spatially encode the signals from the body. The MRI imaging device 150-11 transmits and receives RF signals to and from the body, which are used to create the images. The MRI imaging device 150-11 controls the timing and sequencing of the RF and gradient pulses and processes the signals received from the RF coils. The MRI image device 150-11 controls the acquisition, storage, and processing of the MRI data. The patient lies on a table that moves into the magnet's bore during the scan. The MRI image device 150-11 keeps the magnet and other components at the appropriate temperature. In some embodiments, the MRI imaging device 150-11 may use radio waves to excite the hydrogen atoms in the body through a predefined frequency range, such as between 1 and 100 MHz. In some embodiments, the exact frequency used depends on the strength of the magnetic field applied and other factors, such as the type of tissue being imaged. In some embodiments, the strength of the magnetic field is measured in units of Tesla (T), and the MRI imaging device 150-11 may operate at field strengths of 1.5 T or 3 T. Even higher radio frequencies may be used at higher field strengths, such as 7 T or 10 T. In some embodiments, the frequencies used by the MRI imaging device 150-11 may be lower than those used in other medical imaging techniques, such as X-rays and CT scans, which use ionizing radiation and much higher frequencies, resulting in a safer alternative for certain types of imaging, particularly for patients who may be sensitive to radiation. In some embodiments, the MRI imaging device 150-11 may be best suited for imaging soft tissues and organs and can penetrate several centimeters into the body. The exact penetration depth depends on the strength of the magnetic field used and the specific imaging technique. The effective depth of radiofrequency penetration as derived drops from 17 cm at 85 MHz to 7 cm at 220 MHz. In some embodiments, the MRI imaging device 150-11 may be used to detect and evaluate cancer in the body, making it a valuable tool for cancer diagnosis, staging, and treatment planning. The ability of the MRI imaging device 150-11 to measure cancer depends on several factors, including the type and location of cancer, the imaging protocol used, and the sensitivity and specificity of the MRI technique. In some embodiments, the MRI imaging device 150-11 may be particularly useful for imaging soft tissues and can provide detailed images of internal organs, making it well-suited for detecting tumors in organs such as the brain, breast, prostate, and liver. The MRI imaging device 150-11 may be used to assess the size and location of a tumor, as well as its proximity to surrounding tissues.

Further, embodiments may include a console 152-11, which may be a computer system used to control and monitor the MRI image device 150-11. The console 152-11 may include a processor, memory, storage devices, and input/output interfaces. In some embodiments, the console 152-11 may also include a display, a keyboard, a mouse, and other input/output devices. The console 152-11 controls the acquisition, processing, and display of the MRI images. The console 152-11 may include modules for image acquisition, image reconstruction, image processing, and image display. The console 152-11 may also include software for controlling the scanner hardware, such as the gradient coils, RF coils, and other components. In some embodiments, the console 152-11 may also include a user interface that allows the operator to interact with the system. The operator may use the console 152-11 to adjust scan parameters, initiate scans, monitor the progress of scans, and review the resulting images. The console 152-11 may be connected to the scanner through a network or other communication link. The console 152-11 may communicate with the scanner software to control the scanner hardware and exchange data between the scanner and the console 152-11. The console 152-11 may use the cloud 148-11 to connect to the device 108-11 to exchange data.

Further, embodiments may include a power supply 154-11, which may be used to convert electrical power from a source to a specific form or voltage used by the MRI imaging device 150-11. The power supply 154-11 may be designed to regulate and control the output power to ensure that the MRI imaging device 150-11 receives the correct amount of power without any damage. The power supply 154-11 may include components such as transformers, rectifiers, filters, voltage regulators, and control circuits that work together to provide the desired output voltage and current. The power supply 154-11 may be from an AC or DC power source such as a battery, wall outlet, or generator. The power supply 154-11 may be classified based on various parameters such as output voltage type, power rating, efficiency, regulation, and application. In some embodiments, the power supply 154-11 may include various protection mechanisms such as overvoltage, overcurrent, short-circuit, and thermal protection to ensure safe and reliable operation.

Further, embodiments may include an MRI processor 156-11, also known as a central processing unit (CPU), which may facilitate the operation of the MRI imaging device 150-11 according to the instructions stored in the memory. The MRI processor 156-11 may include suitable logic, circuitry, interfaces, and/or code that may be configured to execute a set of instructions stored in the memory. The MRI processor 156-11 may be a hardware component that performs arithmetic, logic, and control operations on data. The MRI processor 156-11 may be comprised of an arithmetic logic unit, control unit, memory subsystems, and other subsystems. The MRI processor 156-11 may be responsible for performing arithmetic and logical operations on data. The MRI processor 156-11 may include components for addition, subtraction, multiplication, and division and logical operations such as AND, OR, and NOT. The MRI processor 156-11 may be responsible for fetching instructions from memory, decoding them, and executing them. The MRI processor 156-11 may manage the flow of data between different system components, ensuring that operations are performed in the correct order and that data is transferred efficiently. The MRI processor 156-11 may provide fast access to frequently used data and instructions. The MRI processor 156-11 may include components such as caches, registers, and pipelines, which are designed to minimize the time required to access and manipulate data. The MRI processor 156-11 may include various other components and subsystems, such as instruction set architecture (ISA), which may define the set of instructions that the MRI processor 156-11 can execute. The MRI processor 156-11 may specify the format of instructions and data, the addressing modes used to access memory and I/O devices, and the interrupt and exception handling mechanisms used to manage errors and other events. The MRI processor 156-11 may include advanced instruction execution capabilities, support for virtualization and parallel processing, and power management mechanisms that reduce energy consumption and heat dissipation.

Further, embodiments may include an MRI module 158-11, which begins by connecting to the device 108-11. The MRI module 158-11 continuously polls to receive the location data from the location module 138-11. The MRI module 158-11 receives the location data from the location module 138-11. The MRI module 158-11 captures the MRI image of the location. The MRI module 158-11 stores the MRI image data in the MRI database 160-11. The MRI module 158-11 sends the MRI image data to the base module 134-11 and then returns to continuously polling to receive the location data from the location module 138-11.

Further, embodiments may include an MRI database 160-11 which contains the patient ID, such as JS1234, the first name of the patient, such as Jane, the last name of the patient, such as Smith, the area in which the MRI was taken, such as the left breast, and the data files, such as JS-LeftBreast #1 .JPEG. The MRI database 160-11 contains the MRI data of the patient. For example, the MRI may be a medical imaging technique that uses a magnetic field and computer-generated radio waves to create detailed images of the organs and tissues in the body. Most MRI imaging devices 150-11 are large, tube-shaped magnets. When a patient lies inside an MRI imaging device 150-11, the magnetic field temporarily realigns water molecules in the body. Radio waves cause these aligned atoms to produce faint signals, which are used to create cross-sectional MRI images. In some embodiments, the MRI imaging device 150-11 may produce 3D images that can be viewed from different angles. In some embodiments, the MRI database 160-11 contains a series of cross-sectional MRI images and stores the data in the sequence captured by the MRI imaging device 150-11. In some embodiments, the MRI database 160-11 may contain the location data received from the device 108-11.

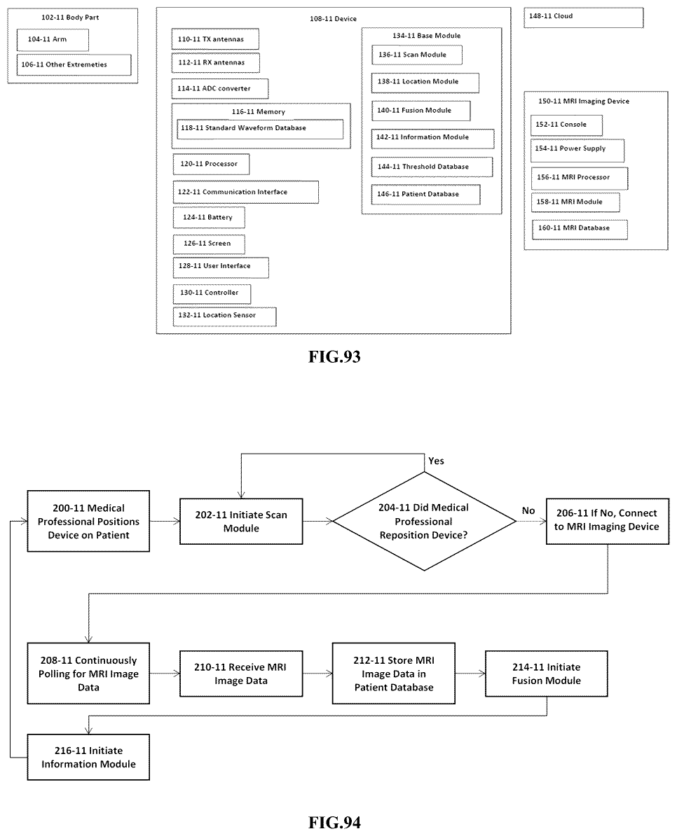

FIG. 94 illustrates an example operation of the base module 134-11. The process begins with the medical professional positioning, at step 200-11, the device 108-11 on the patient. The medical professional positions, places, locates, etc., the device 108-11 on the patient's body in an area or region of interest to collect data, such as potential sites of inflammation which may indicate cancer. The base module 134-11 initiates, at step 202-11, the scan module 136-11. The scan module 136-11 begins by being initiated by the base module 134-11. The scan module 136-11 sends the RF transmit signal to the TX antennas 110-11. The scan module 136-11 stores the RF transmit signal to memory 116-11. The scan module 136-11 receives the RF signal from the RX antennas 112-11. The scan module 136-11 converts to digital using the ADC converter 114-11. The scan module 136-11 stores the RX converted signal data in memory 116-11. The scan module 136-11 correlates the RF signals with ground truth data to determine the analyte levels. The scan module 136-11 stores the analyte levels in the patient database 146-11. The scan module 136-11 initiates the location module 138-11. The scan module 136-11 compares analyte levels to the threshold database 144-11. The scan module 136-11 determines if the analyte levels are over the thresholds stored in the threshold database 144-11. If it is determined that the analyte levels are over the thresholds stored in the threshold database 144-11, the scan module 136-11 connects to the MRI module 158-11. The scan module 136-11 extracts the analyte levels and the location data from the patient database 146-11. The scan module 136-11 sends the analyte levels and the location data to the MRI module 158-11. If it is determined that the analyte levels are not over the thresholds stored in threshold database 144-11 or after the data is sent to the MRI module 158-11, the scan module 136-11 returns to the base module 134-11. The base module 134-11 determines, at step 204-11, if the medical professional repositioned the device 108-11. If it is determined that the medical professional repositioned the device 108-11, the base module 134-11 returns to initiating the scan module 136-11. The base module 134-11 may determine if the medical professional repositioned the device 108-11 by continuously monitoring the location sensor 132-11 to determine if the position, such as the X, Y, and Z coordinates, of the device 108-11 have changed. In some embodiments, the base module 134-11 may receive an alert from the location sensor 132-11 if any of the location data has changed, indicating that the device 108-11 has been repositioned. If it is determined that the medical professional did not reposition the device 108-11, the base module 134-11 connects, at step 206-11, to the MRI imaging device 150-11. If the device 108-11 has not been repositioned or if the device 108-11 has been removed completely from the patient's body, the base module 134-11 connects to the MRI imaging device 150-11. The base module 134-11 may initiate the scan module 136-11, and the base module 134-11 may receive a signal that the location module 138-11 has been initiated, and an alert may be provided to the medical professional to not move the device 108-11 until the location data has been sent to the MRI imaging device 150-11. Once the location has been sent, the base module 134-11 may receive an alert to remove the device 108-11 from the patient so that the MRI image may be captured, and the device 108-11 connects to the MRI imaging device 150-11 to receive the MRI image data. The base module 134-11 continuously polls at step 208-11 to receive the MRI image data from the MRI imaging device 150-11. If the device 108-11 has not been repositioned or if the device 108-11 has been removed completely from the patient's body, the base module 134-11 connects to the MRI imaging device 150-11. The base module 134-11 may initiates the scan module 136-11, and the base module 134-11 may receive a signal that the location module 138-11 has been initiated, and an alert may be provided to the medical professional to not move the device 108-11 until the location data has been sent to the MRI imaging device 150-11. Once the location has been sent, the base module 134-11 may receive an alert to remove the device 108-11 from the patient so that the MRI image may be captured, and the device 108-11 connects to the MRI imaging device 150-11 to receive the MRI image data. The base module 134-11 receives, at step 210-11, the MRI image data from the MRI imaging device 150-11. Once the location has been sent, the base module 134-11 may receive an alert to remove the device 108-11 from the patient so that the MRI image may be captured, and the device 108-11 connects to the MRI imaging device 150-11 to receive the MRI image data. For example, the base module 134-11 may receive MRI image data related to the patient, such as the patient ID, such as JS1234, the first name of the patient, such as Jane, the last name of the patient, such as Smith, the area in which the MRI was taken, such as the left breast, and the data files, such as JS-LeftBreast #1 .JPEG. The base module 134-11 stores, at step 212-11, the MRI image data in the patient database 146-11. The base module 134-11 may store the MRI image data related to the patient, such as the patient ID, such as JS1234, the first name of the patient, such as Jane, the last name of the patient, such as Smith, the area in which the MRI was taken, such as the left breast, and the data files, such as JS-LeftBreast #1 .JPEG. The base module 134-11 initiates, at step 214-11, the fusion module 140-11. The fusion module 140-11 begins by being initiated by the base module 134-11. The fusion module 140-11 extracts the analyte data and MRI image data from the patient database 146-11. The fusion module 140-11 performs the fusion model on the data. The fusion module 140-11 stores the results from the fusion model in the patient database 146-11. The fusion module 140-11 returns to the base module 134-11. The base module 134-11 initiates, at step 216-11, the information module 142-11. The information module 142-11 begins by being initiated by the base module 134-11. The information module 142-11 extracts the fusion model results from patient database 146-11. The information module 142-11 displays the fusion model results data on the user interface 128-11. The information module 142-11 returns to the base module 134-11.

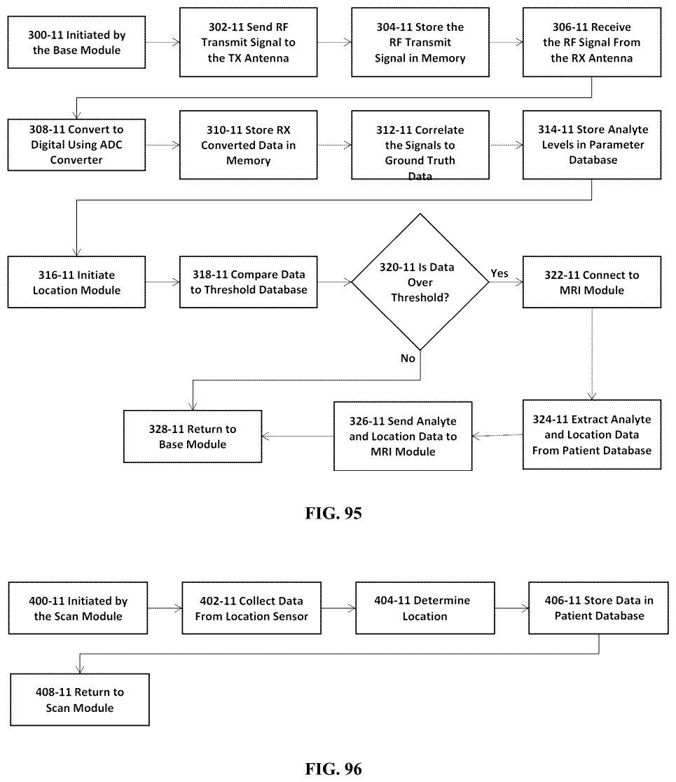

FIG. 95 illustrates an example operation of the scan module 136-11. The process begins with the scan module 136-11 being initiated, at step 300-11, by the base module 134-11. In some embodiments, the scan module 136-11 may be initiated when the device 108-11 has not moved, or been repositioned, in a predetermined amount of time, such as 15 seconds. The scan module 136-11 sends, at step 302-11, the RF transmit signal to the TX antennas 110-11. The one or more TX antennas 110-11 may be configured to transmit the Activated RF range signals at a predefined frequency. In one embodiment, the predefined frequency may correspond to a range suitable for the human body. For example, the one or more TX antennas 110-11 transmit Activated RF range signals at a range of 500 MHz to 300 GHz. The scan module 136-11 stores, at step 304-11, the RF transmit signal to memory 116-11. The scan module 136-11 stores the transmitted signal to memory 116-11, such as Activated RF range signals at a range of 500 MHZ to 300 GHz. The scan module 136-11 receives, at step 306-11, the RF signal from the RX antennas 112-11. The one or more RX antennas 112-11 may be configured to receive the reflected portion of the Activated RF range signals. In one embodiment, the Activated RF range signals may be transmitted to the user's skin, and electromagnetic energy may be reflected from many parts, such as fibrous tissue, muscle, tendons, bones, and the skin. Further, the electromagnetic energy reflected from the blood molecules may be received by the one or more RX antennas 112-11. In some embodiments, the non-invasive RF device 108-11 may include RF sensors which may be used to measure inflammation in the body. RF sensors may be used for cancer detection, particularly in the context of detecting circulating tumor cells (CTCs) or other biomarkers associated with cancer. The scan module 136-11 converts, at step 308-11, to digital using the ADC converter 114-11. For example, the ADC may be configured to convert the Activated RF range signals from an analog signal into a digital processor readable format. The scan module 136-11 stores, at step 310-11, the RX converted signal data in memory 116-11. The scan module 136-11 stores the received signal from the RX antennas 112-11 that has been converted to a digital processor readable format in memory 116-11. The scan module 136-11 correlates, at step 312-11, the RF signals with ground truth data to determine the analyte levels. The scan module 136-11 may be configured to execute an AI correlation between the real-time ground truth data and the RX converted data. In one embodiment, the AI correlation between the real-time ground truth data and the RX-converted data is executed to determine whether the RX-converted data corresponds to the real-time ground truth data. The ground truth data may be determined by a medical device 108-11 that identifies the analyte levels or number at the time a waveform was transmitted and received. Machine learning processes may be performed to identify specific analyte waveforms, in which complex responded signals from stepped frequencies transmit signals that can be related to analyte levels. The memory 116-11 is used in real-time to compare received waveforms from the RX antenna 112-11 to a standard waveform database 118-11 stored in memory 116-11 to identify the analyte number for the received waveform from the RX antenna 112-11. In one embodiment, the standard waveform database 118-11 may be configured to store the filtered RF signal received from the one or more RX antennas 112-11 of the device 108-11. The standard waveform database 118-11 may store the signal waveforms for the TX antenna 110-11 and the received signal waveforms for the RX antenna 112-11. The database may include the analyte readings with the corresponding signal waveform, received waveform and the TX antenna 110-11 and RX antenna 112-11 that were used. For example, the standard waveform database 118-11 may include raw or converted device 108-11 readings from patients known to have inflammation due to cancerous cells or tumors or an average of multiple patients. This data can be compared to readings from a person with an unknown condition in order to determine if the waveforms taken from that person match any of the known standard waveforms. The standard waveform database 118-11 may contain the transmitted RF signals by the TX antennas 110-11, the received RF signals by the RX antennas 112-11, the analyte associated with signals, and the measurement of the analyte, such as 5 CTCs, or circulating tumor cells, per 7.5 ml. The standard waveform database 118-11 may contain a plurality of historical data entries of historical patients with the RF signals, both transmitted and received, the analyte, and the analyte levels. For example, the analytes may be CTCs which would indicate cancerous cells in the bloodstream, C-reactive Protein, interleukin-6, or tumor necrosis factor-alpha, which would be an indicator of inflammation that may be caused by cancer. The scan module 136-11 stores, at step 314-11, the analyte number in the patient database 146-11. The scan module 136-11 stores the analyte data in the patient database 146-11, such as the name of the analyte, the analyte levels, etc., stored in a data file. The scan module 136-11 initiates, at step 316-11, the location module 138-11. The location module 138-11 begins by being initiated by the scan module 136-11. The location module 138-11 collects the location data from the location sensor 132-11. The location module 138-11 determines the location of the device 108-11. The location module 138-11 stores the location data in the patient database 146-11. The location module 138-11 returns to the scan module 136-11. The scan module 136-11 compares, at step 318-11, analyte levels to the threshold database 144-11. The scan module 136-11 compares the analyte levels to determine if there is an indication of inflammation in the area which would prompt the system to send the location of the device 108-11 to the MRI image device 150-11 to capture an MRI image. The threshold database 144-11 contains thresholds for various analyte levels that are compared to the analyte levels collected by the device 108-11 during the scan module 136-11 in order to determine if an MRI image should be taken of the area. The threshold database 144-11 may contain the type of analyte and a threshold that indicates potential inflammation in the area. For example, the levels of certain analytes can be elevated or reduced in response to inflammation or other pathological processes in the body. C-reactive protein (CRP) is an analyte commonly measured in the blood to detect inflammation as its levels increase in response to inflammation. Circulating tumor cells, or CTCs, are cancer cells from a tumor traveling in the bloodstream. CTCs provide genetic information about cancer, which can provide the tumor's location, inform medical professionals about the best treatments, etc. CTCs and their clusters disseminate to distant bodily sites through the bloodstream as part of the metastatic process. Their occurrence is rare, often<100 CTCs or <10 clusters per 10× 106 leukocytes and 5×109 erythrocytes in 1 ml whole blood. Nevertheless, their occurrence far exceeds the number of actual metastatic lesions in patients, supporting the notion that very few CTCs overcome the harsh vascular environment and successfully metastasize onto a secondary site. Other analytes, such as cytokines or chemokines, can also be indicators of inflammation or other immune responses. The analyte levels or concentrations may provide important information about the presence and severity of inflammation or other pathological processes in the body, such as cancer. C-reactive protein (CRP), circulating cancer cells, cytokines, and chemokines may be used to detect cancer-related inflammation in the blood. For example, CRP is an acute-phase protein produced in response to inflammation, including cancer-related inflammation. Elevated levels of CRP in the blood are often seen in patients with various types of cancer, including lung, breast, colorectal, and pancreatic cancer. Levels greater than 10 mg/L are generally considered to indicate the presence of acute inflammation. Also, cytokines and chemokines are signaling molecules that play important roles in immune responses and inflammation. Some cytokines and chemokines are produced by cancer cells or immune cells in response to cancer, and their levels in the blood can be used to detect cancer-related inflammation. Interleukin-6 (IL-6) is a cytokine often elevated in patients with cancer, particularly those with advanced disease, and its levels can be used to monitor treatment response and predict prognosis. A cytokine, such as interleukin, may have levels greater than 100 μg/mL, indicating patients with severe infections or other inflammatory conditions. Another cytokine, such as tumor necrosis factor-alpha, may have levels greater than 100 μg/mL, indicating patients with sepsis, autoimmune diseases, or other inflammatory conditions. Lastly, the detection of CTCs in the bloodstream indicates the presence of tumor cells in the bloodstream, which can indicate active cancer in the body. The presence of 5 CTCs per 7.5 millimeters of blood would indicate an increased risk of cancer and a poorer prognosis. The scan module 136-11 determines, at step 320-11, if the analyte levels data is over the thresholds stored in the threshold database 144-11. C-reactive protein (CRP), cytokines, and chemokines may be used to detect cancer-related inflammation in the blood. CRP is an acute-phase protein produced in response to inflammation, including cancer-associated inflammation. Elevated levels of CRP in the blood are often seen in patients with various types of cancer, including lung, breast, colorectal, and pancreatic cancer. Levels greater than 10 mg/L are generally considered to indicate the presence of acute inflammation. Also, cytokines and chemokines are signaling molecules that play important roles in immune responses and inflammation. Some cytokines and chemokines are produced by cancer cells or immune cells in response to cancer, and their levels in the blood can be used to detect cancer-related inflammation. Interleukin-6 (IL-6) is a cytokine often elevated in patients with cancer, particularly those with advanced disease, and its levels can be used to monitor treatment response and predict prognosis. A cytokine such as interleukin may have levels greater than 100 μg/mL, indicating patients with severe infections or other inflammatory conditions. Another cytokine, such as tumor necrosis factor-alpha, may have levels greater than 100 μg/mL, indicating patients with sepsis, autoimmune diseases, or other inflammatory conditions. If it is determined that the analyte levels data is over the thresholds stored in the threshold database 144-11, the scan module 136-11 connects, at step 322-11, to the MRI module 158-11. The scan module 136-11 may connect to the MRI module 158-11 through the cloud 148-11. The scan module 136-11 extracts, at step 324-11, the analyte levels data and the location data from the patient database 146-11. The scan module 136-11 extracts the data to be provided to the MRI module 158-11 to capture an MRI image of a region of interest on the patient's body. The location data may be the device 108-11 location at the time of the readings, which may be a region of the patient's body, a specific body part 102-11, or an exact location of where the device 108-11 was located on the patient's body. The scan module 136-11 sends, at step 326-11, the analyte levels data and the location data to the MRI module 158-11. The location data may be the device 108-11 location at the time of the readings, which may be a region of the patient's body, a specific body part 102-11, or an exact location of where the device 108-11 was on the patient's body. If it is determined that the analyte levels data is not over the thresholds stored in threshold database 144-11 or after the data is sent to the MRI module 158-11, the scan module 136-11 returns, at step 328-11, to the base module 134-11.

FIG. 96 illustrates an example operation of the location module 138-11. The process begins with the location module 138-11 being initiated, at step 400-11, by the scan module 136-11. The location module 138-11 may be initiated once the scan module 136-11 has determined that the analyte levels are over or below the thresholds stored in the threshold database 144-11, and the location of the device 108-11 is required to assist the MRI module 158-11 in capturing an MRI image of the desired area of the patient. The location module 138-11 collects, at step 402-11, the location data from the location sensor 132-11. The location module 138-11 collects the location data from the location sensor 132-11, which may include an accelerometer, gyroscopes, and magnetometers, which may provide a comprehensive measurement of the device's 108-11 positions and orientation. The location module 138-11 determines, at step 404-11, the location of the device 108-11. The location module 138-11 may use data from the location sensor 132-11, such as an accelerometer, which may be used to measure acceleration or changes in motion. The location sensor 132-11 may consist of a mass that is suspended by springs, which is able to move in response to changes in acceleration. The motion of the mass is detected by one or more sensors, which convert the motion into an electrical signal that can be processed and analyzed. The sensors used in the location sensor 132-11 may include piezoelectric sensors, capacitive sensors, or optical sensors, etc. The location sensor 132-11 may be used to determine the device's 108-11 location on the patient's body. For example, the location sensor 132-11 is integrated into the device 108-11 in a position that allows it to measure the desired motion or orientation. Then before using the location sensor 132-11 to measure motion or orientation, the location sensor 132-11 is calibrated to ensure accurate readings. The calibration may involve setting the zero point, such as the point where there is no acceleration, and determine the sensitivity of the device 108-11. Then the location sensor 132-11 produces data in the form of acceleration values along the three axes (X, Y, and Z). By integrating these values over time, it determines the velocity and position of the device 108-11. Filtering and signal processing techniques may be used to clean up the data and extract the desired information. Once the data has been processed, it can be analyzed to determine the device's 108-11 position on the body. In some embodiments, the location sensor 132-11 may combine accelerometer data with other sensors, such as gyroscopes and magnetometers, to provide a more comprehensive measurement of the device's 108-11 positions and orientation. The location module 138-11 stores, at step 406-11, the location data in the patient database 146-11. The location data may be the device 108-11 location at the time of the readings, which may be a region of the patient's body, a specific body part 102-11, or an exact location of where the device 108-11 was on the patient's body. In some embodiments, the location data may be the X, Y, and Z coordinates of the device 108-11 at the time of the readings or analyte levels were captured. The location module 138-11 returns, at step 408-11, to the scan module 136-11.

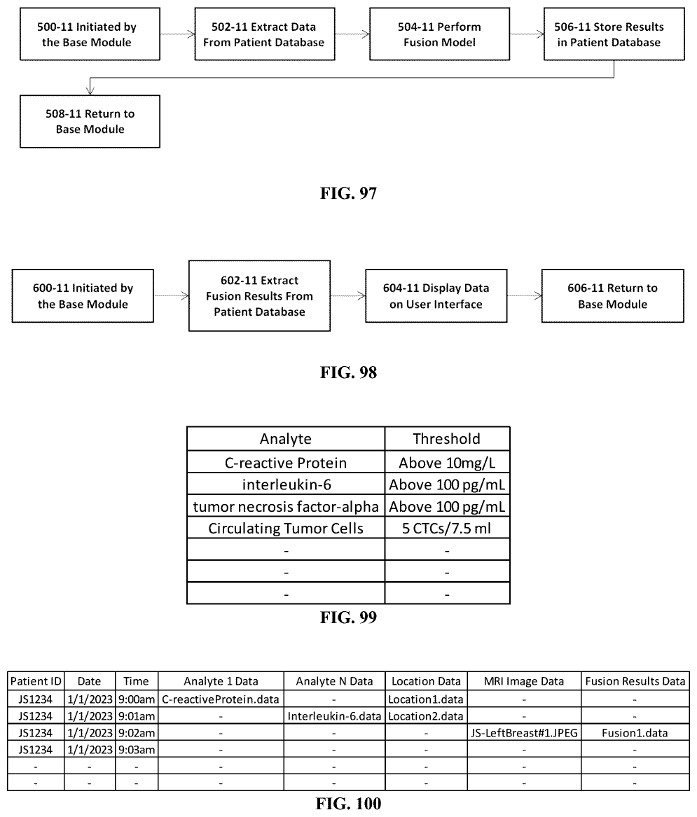

FIG. 97 illustrates an example operation of the fusion module 140-11. The process begins with the fusion module 140-11 being initiated, at step 500-11, by the base module 134-11. Once the base module 134-11 receives and stores the MRI image data from the MRI module 158-11, the fusion module 140-11 may be initiated to perform the fusion model and store the results in the patient database 146-11 to display the data to the medical professional. The fusion module 140-11 extracts, at step 502-11, the analyte data and MRI image data from the patient database 146-11. The fusion module 140-11 may extract the data stored in the patient database 146-11, such as the data collected from the device 108-11, MRI imaging device 150-11, and the results of the fusion model. The patient database 146-11 may contain patient ID, date, time, a first analyte, an analyte “N” indicating an infinite number of potential analyte readings, the location data, the MRI image data, and the fusion model results. The patient database 146-11 may contain the analyte data, such as the analyte levels or readings captured from the device 108-11, stored as a data file which may include information such as the type of analyte, the analyte readings, etc. The patient database 146-11 may contain the location data such as the device 108-11 location at the time of the readings, which may be a region of the patient's body, a specific body part 102-11, or an exact location of where the device 108-11 was located on the patient's body. The MRI image data may be 3D images that can be viewed from different angles. The fusion model results may be a data file that contains the analyte readings data, and the MRI image data overlayed on one another to provide a healthcare provider with a graphical representation of the data, such as the MRI image data with the location of the device 108-11 and the analyte readings in the section of the device 108-11. The fusion module 140-11 performs, at step 504-11, the fusion model on the data. The fusion model may be an algorithm to calculate a patient's cancer probability using the collected RF data from device 108-11 and the MRI image data. For example, the fusion model may use the data from the MRI image, such as if a tumor was identified, the size of the tumor, the shape of the tumor, etc., and the collected RF data, such as the locations of the device when the analytes associated with cancer were detected to determine the spread of cancerous cells, or potentially cancerous cells, throughout the region near the tumor. The resulting probability of the fusion model may be a positive or negative prognosis for the patient, the stage of the cancer, the aggressiveness of the cancer, etc. The fusion model may use historical patient data, such as historical images of tumors, historical analyte levels, etc., to create the probability of cancer. For example, if a small tumor is detected by the MRI imaging device 150-11 and the RF device only detects CTCs around the tumor and does not detect any other analytes associated with inflammation due to cancer in various other locations of the body, the result of the fusion model may be cancer prognosis is positive, early stages, by comparing the data collected from the patient to historical patients with similar data points to determine the prognosis. The fusion model may be an algorithm to detect cancer in a patient, similar to an image-guided biopsy, by using the collected MRI image data to determine the location of where the device 108-11 should be placed to collect the RF data to determine if there are any CTCs, analytes associated with inflammation, etc. For example, the fusion model may receive the MRI image data, which may detect a lump, tumor, growth, etc., in the patient's left breast. Then the medical professional may place the device 108-11 around the area of the tumor to collect the RF data, such as detecting CTCs, and if the collected RF data results in the patient having more than 5 CTCs per 7.5 milliliters, it may be determined that the tumor is cancerous. In some embodiments, the fusion model may be used to display the collected data to the medical professional to allow the medical professional to review the data. For example, the fusion model may use the MRI image data and the place the locations of the device 108-11 on the image data in the locations where analytes associated with cancer were detected, such as CTCs, analytes associated with inflammation, etc. The image may display the tumor, growth, lump, etc., on the left breast of the patient, and then locations of the device 108-11 in which the analytes associated with cancer were detected may be marked, labeled, or otherwise displayed on the image to allow the medical professional to review the collected data. The fusion module 140-11 stores, at step 506-11, the results from the fusion model in the patient database 146-11. The fusion module 140-11 stores the results of the fusion model, such as the combined images, in the patient database 146-11. The fusion module 140-11 returns, at step 508-11, to the base module 134-11.

FIG. 98 illustrates an example operation of the information module 142-11. The process begins with the information module 142-11 being initiated, at step 600-11, by the base module 134-11. In some embodiments, the information module 142-11 may be initiated once the fusion model results are stored in the patient database 146-11. The information module 142-11 extracts, at step 602-11, the fusion model results from patient database 146-11. The information module 142-11 extracts the results of the fusion model, such as the combined images, from the patient database 146-11. The information module 142-11 displays, at step 604-11, the fusion model results data on the user interface 128-11. For example, the medical professional would be able to view an image that displays the MRI image data as well as the analyte RF data as it is dispersed throughout the patient's body. The information module 142-11 returns, at step 606-11, to the base module 134-11.

FIG. 99 illustrates the threshold database 144-11. The threshold database 144-11 may contain thresholds for various analyte levels that are compared to the analyte levels collected by the device 108-11 during the scan module 136-11 in order to determine if an MRI image should be taken of the area. The threshold database 144-11 may contain the type of analyte and a threshold which indicates potential inflammation in the area. For example, the levels of certain analytes can be elevated or reduced in response to inflammation or other pathological processes in the body. C-reactive protein (CRP) is an analyte commonly measured in the blood to detect inflammation as its levels increase in response to inflammation. Circulating tumor cells, or CTCs, are cancer cells from a tumor traveling in the bloodstream. CTCs provide genetic information about cancer, which can provide the tumor's location, inform medical professionals about the best treatments, etc. CTCs and their clusters disseminate to distant bodily sites through the bloodstream as part of the metastatic process. Their occurrence is rare, often<100 CTCs or <10 clusters per 10×106 leukocytes and 5×109 erythrocytes in 1 ml whole blood. Nevertheless, their occurrence far exceeds the number of actual metastatic lesions in patients, supporting the notion that very few CTCs overcome the harsh vascular environment and successfully metastasize onto a secondary site. Other analytes, such as cytokines or chemokines, can also be indicators of inflammation or other immune responses. The analyte levels or concentrations may provide important information about the presence and severity of inflammation or other pathological processes in the body, such as cancer. C-reactive protein (CRP), circulating cancer cells, cytokines, and chemokines may be used to detect cancer-related inflammation in the blood. For example, CRP is an acute-phase protein produced in response to inflammation, including inflammation associated with cancer. Elevated levels of CRP in the blood are often seen in patients with various types of cancer, including lung, breast, colorectal, and pancreatic cancer. Levels greater than 10 mg/L are generally considered to indicate the presence of acute inflammation. Also, cytokines and chemokines are signaling molecules that play important roles in immune responses and inflammation. Some cytokines and chemokines are produced by cancer cells or immune cells in response to cancer, and their levels in the blood can be used to detect cancer-related inflammation. Interleukin-6 (IL-6) is a cytokine often elevated in patients with cancer, particularly those with advanced disease, and its levels can be used to monitor treatment response and predict prognosis. A cytokine, such as interleukin, may have levels greater than 100 μg/mL, indicating patients with severe infections or other inflammatory conditions. Another cytokine, such as tumor necrosis factor-alpha, may have levels greater than 100 μg/mL, indicating patients with sepsis, autoimmune diseases, or other inflammatory conditions. Lastly, the detection of CTCs in the bloodstream indicates the presence of tumor cells in the bloodstream, which can indicate active cancer in the body. The presence of 5 CTCs per 7.5 millimeters of blood would indicate an increased risk of cancer and a poorer prognosis.

FIG. 100 illustrates the patient database 146-11. The database may contain the data collected from the device 108-11, MRI imaging device 150-11, and the results of the fusion model. The patient database 146-11 may contain patient ID, date, time, a first analyte, an analyte “N” indicating an infinite number of potential analyte readings, the location data, the MRI image data, and the fusion model results. The patient database 146-11 may contain the analyte data, such as the analyte levels or readings captured from the device 108-11, stored as a data file which may include information such as the type of analyte, the analyte readings, etc. The patient database 146-11 may contain the location data such as the device 108-11 location at the time of the readings, which may be a region of the patient's body, a specific body part 102-11, or an exact location of where the device 108-11 was located on the patient's body. The MRI image data may be 3D images that can be viewed from different angles. The fusion model results may be a data file that contains the analyte readings data, and the MRI image data overlayed on one another to provide a healthcare provider with on graphical representation of the data, such as the MRI image data with the location of the device 108-11 and the analyte readings in the section of the device 108-11.

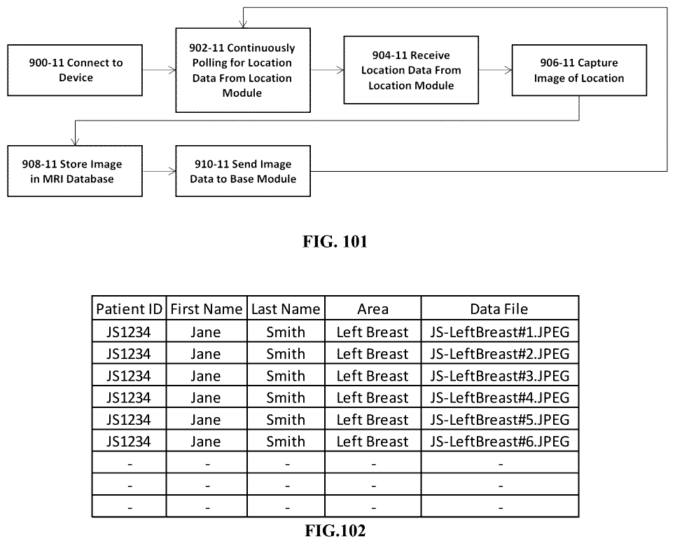

FIG. 101 illustrates an example operation of the MRI module 158-11. The process begins with the MRI module 158-11 connecting, at step 900-11, to the device 108-11. The MRI module 158-11 may connect to the device 108-11 through the cloud 148-11. The MRI module 158-11 continuously polls at step 902-11 to receive the location data from the location module 138-11. The MRI module 158-11 is continuously polling to receive the location data from the location module 138-11 to determine the region of the body that is of interest for an MRI image. The MRI module 158-11 receives, at step 904-11, the location data from the location module 138-11. The MRI module 158-11 receives the location data, such as the device 108-11 location at the time of the readings, which may be a region of the patient's body, a specific body part 102-11, or an exact location of where the device 108-11 was located on the patient's body, etc. which would indicate a potential area of the body that would require an MRI image for further assessment. The MRI module 158-11 captures, at step 906-11, the MRI image of the location. The MRI module 158-11 captures an MRI image of the region of the body or body part 102-11 indicated by the location data. The MRI module 158-11 stores, at step 908-11, the MRI image data in the MRI database 160-11. The MRI module 158-11 may store patient-related data, such as the patient ID, such as JS1234, the first name of the patient, such as Jane, the last name of the patient, such as Smith, the area in which the MRI was taken, such as the left breast, and the data files, such as JS-LeftBreast #1 .JPEG. The MRI module 158-11 sends, at step 910-11, the MRI image data to the base module 134-11 and then returns to continuous polling to receive the location data from the location module 138-11.

FIG. 102 illustrates the MRI database 160-11. The database contains the patient ID, such as JS1234, the patient's first name, such as Jane, and the patient's last name, such as Smith. The area in which the MRI was taken, such as the left breast, and the data files, such as JS-LeftBreast #1 .JPEG. The MRI database 160-11 contains the MRI data of the patient. For example, the MRI may be a medical imaging technique that uses a magnetic field and computer-generated radio waves to create detailed images of the organs and tissues in the body. Most MRI imaging devices 150-11 are large, tube-shaped magnets. When a patient lies inside an MRI imaging device 150-11, the magnetic field temporarily realigns water molecules in the body. Radio waves cause these aligned atoms to produce faint signals, which are used to create cross-sectional MRI images. In some embodiments, the MRI imaging device 150-11 may produce 3D images that can be viewed from different angles. In some embodiments, the MRI database 160-11 contains the series of cross-sectional MRI images and stores the data in the sequence captured by the MRI imaging device 150-11. In some embodiments, the MRI database 160-11 may contain the location data received from the device 108-11.

In one example implementation of Embodiment 11, a system can include an MRI device; a non-invasive RF device; a memory; a controller; and a fusion module in the memory; wherein the MRI device and the non-invasive RF device are controlled using the controller to put data collected by the MRI device and the non-invasive RF device in the memory and then execute the fusion module to fuse the collected data of both the MRI device and the non-invasive RF device.

### Embodiment 12

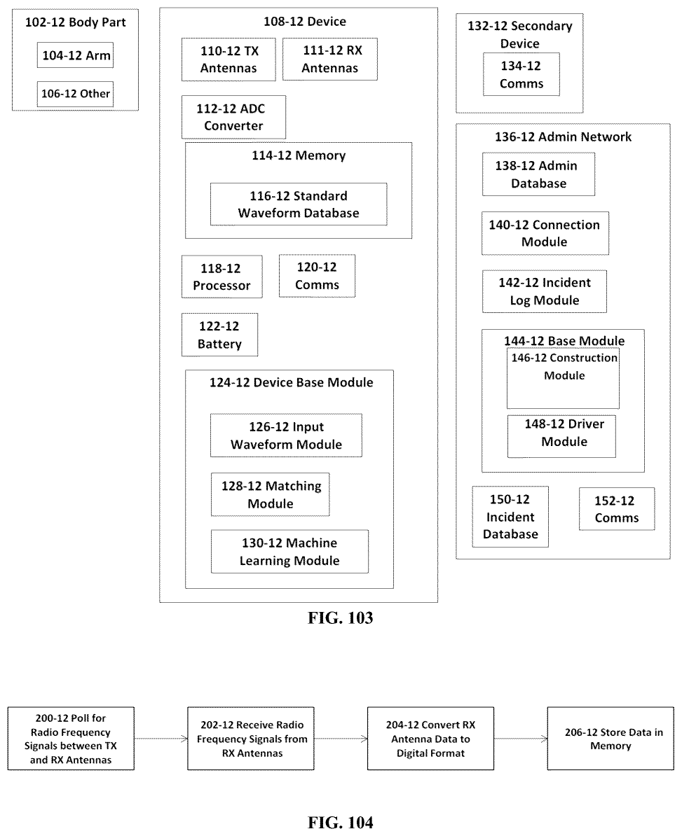

FIG. 103 is a schematic illustration of a system for radio frequency health monitoring. This system is configured to be attached to or in proximity to a body part 102-12. The body part 102-12 may be an arm 104-12. The body part 102-12 may be another body part 106-12 besides an arm, such as a leg, finger, chest, head, or any other body part from which useful medical parameters can be taken. The system may comprise a device 108-12, which may be a wearable and portable device such as, but not limited to, a cell phone, a smartwatch, a tracker, a wearable monitor, a wristband, and a personal blood monitoring device.

The system may further comprise one or more transmit (“TX”) antennas 110-12. The one or more TX antennas 110-12 may be configured to transmit RF in the RF Activated Range from 500 MHZ to 300 GHZ. A pre-defined frequency may correspond to a range suitable for the human body in one embodiment. For example, the one or more TX antennas 110-12 would transmit radio frequency signals at a range of 120-126 GHz.

The system may further comprise one or more receive (“RX”) antennas 111-12. The one or more RX antennas 111-12 may be configured to receive the RF signals in response to the RF signal transmitted from the one or more TX antennas 110-12.

The system may further comprise an ADC converter 112-12, which may be configured to convert the received RF signals from an analog signal into a digital processor readable format.

The system may further comprise memory 114-12, which may be configured to store the transmitted RF signals by the one or more TX antennas 110-12 and store the received portion of the response or responded RF signals from the one or more RX antennas 111-12. Further, the memory 114-12 may also store the converted digital processor readable format by the ADC converter 112-12. The memory 114-12 may include suitable logic, circuitry, and/or interfaces that may be configured to store a machine code and/or a computer program with at least one code section executable by the processor 118-12. Examples of implementation of the memory 114-12 may include, but are not limited to, Random Access Memory (RAM), Read Only Memory (ROM), Hard Disk Drive (HDD), and/or a Secure Digital (SD) card.

The system may further comprise a standard waveform database 116-12, which may contain standard waveforms for known patterns. These may be raw or converted device readings from patients or persons with known conditions. For example, the standard waveform database 116-12 may include raw or converted device readings from a patient known to have diabetes or an average of multiple patients. This data can be compared to readings from a person with an unknown condition in order to determine if the waveforms taken from that person match any of the known standard waveforms.

The system may further comprise a processor 118-12, which may facilitate the operation of the device 108-12 according to the instructions stored in the memory 114-12. The processor 118-12 may include suitable logic, circuitry, interfaces, and/or code that may be configured to execute a set of instructions stored in the memory 114-12.

The system may further comprise a comms 120-12, which may be a communication element such as a Wi-Fi or RFC transmitter and receiver or physical connection that can send and receive data to other devices of the system and devices external to the system. The comms 120-12 may be configured to comply with regulatory acts such as HIPPA. For example, the comms 120-12 may include features such as encryption, access control, audit trails, secure transmission, credential verification, or other features which may protect the integrity and privacy of transmitted or received data.

The system may further comprise a battery 122-12, which may power hardware modules of the device 108-12. The device 108-12 may be configured with a charging port to recharge the battery 122-12. Charging of the battery 122-12 may be wired or wireless.

The system may further comprise a device base module 124-12, which may be configured to store instructions for executing the computer program from the converted digital processor readable format of the ADC converter 112-12. The device base module 124-12 may be configured to facilitate the operation of the processor 118-12, the memory 114-12, the one or more TX antennas 110-12, the one or more RX antennas 111-12, and the comms 120-12. Further, the device base module 124-12 may be configured to create polling of the RF Activated Range from 500 MHZ to 300 GHZ. It can be noted that the device base module 124-12 may be configured to filter the RF Activated Range from 500 MHZ to 300 GHZ received from one or more RX antennas 111-12.

The system may further comprise an input waveform module 126-12, which may extract a radio frequency waveform from memory. This may be the raw or converted data recording from the one or more RX antennas 111-12 from a patient wearing the device. If the entire radio frequency waveform is too long for effective matching, the input waveform module 126-12 may select a time interval within the data set. This input waveform may then be sent to the matching module 128-12.

The system may further comprise a matching module 128-12, which may match the input waveform and each standard waveform in the standard waveform database 116-12 by performing a convolution and/or cross-correlation of the input waveform and the standard waveform. These convolutions and/or cross-correlations are then sent to the machine learning module.

The system may further comprise a machine learning module 130-12 which has been trained to identify health parameters based on the convolution and/or cross-correlations of the input and standard waveforms. The machine learning module 130-12 receives the convolutions and cross-correlations from the matching module 128-12 and outputs any health parameters identified.

The system may further comprise one or more industrial sensors 132-12. An industrial sensor may be any sensor used in industrial equipment or any sensor used in reducing industrial accidents. Examples of industrial sensors may include accelerometers, barometers, gyroscopes, magnetometers, humidity sensors, temperature sensors, pressure sensors, flow sensors, proximity sensors, optical sensors, and acoustic sensors.

The system may further comprise an industrial sensor comms 134-12, which may be a communication element such as a Wi-Fi or RFC transmitter and receiver or physical connection that can send and receive data to other devices of the system and devices external to the system. The industrial sensor comms 134-12 may be configured to comply with regulatory acts such as HIPPA. For example, the industrial sensor comms 134-12 may include encryption, access control, audit trails, secure transmission, credential verification, or other features that may protect the integrity and privacy of transmitted or received data.

The system may further comprise an admin network 136-12, which may be a computer or network of computers which may send and receive data. The admin network 136-12 may be accessed via an application on a user device such as a PC, smartphone, smartwatch, iPhone, etc. The admin network 136-12 may be an application center or store which allows industrial sensors 132-12 to be registered to communicate with the network. The admin network 136-12 may communicate with other networks, such as third-party networks or other iterations of the admin network 136-12.

The system may further comprise an admin database 138-12, which may store the health parameters output by the machine learning module 130-12 and data from the industrial sensors 132-12.

The system may further comprise a connection module 140-12, which may connect to the device 108-12 and industrial sensors 132-12 and collect data which then may be stored in the admin database 138-12.

The system may further comprise an incident log module 142-12, which may allow a user or administrator of the system to log an incident. The incident log may include the nature of the incident, the time of the incident, and which individuals were involved.

The system may further comprise a base module 144-12, which may initiate the construction module 146-12 and/or driver module 148-12 based on which module the user requires. For example, users in a construction setting would use the construction module 146-12. The system may comprise additional modules for different settings, such as farming, manufacturing, mining, healthcare, etc.

The system may further comprise a construction module 146-12, which may predict imminent construction incidents based on the data from the device 108-12 and industrial sensors 132-12. The construction module 146-12 may determine the risk of accidents and injuries associated with specific ongoing construction activities based on the health conditions of workers. These predictions may be communicated to the worker, a supervisor, and/or a central control station, enabling appropriate intervention to ensure worker safety.

The system may further comprise a driver module 148-12, which may predict imminent driving incidents based on the data from the device 108-12 and industrial sensors 132-12. The driving module 148-12 may determine the risk of road accidents based on the health conditions of a driver. These predictions may be communicated to the driver, a supervisor, and/or a central control station, enabling appropriate intervention to ensure driver safety.

The system may further comprise an incident database 150-12, which may store a record of industrial incidents and associated data from the device 108-12 and industrial 132-12 sensors. This data may be used to recognize data patterns that occur leading up to an incident.

The system may further comprise an admin network comms 152-12, which may be a communication element such as a Wi-Fi or RFC transmitter and receiver or physical connection that can send and receive data to other devices of the system and devices external to the system. The admin network comms 152-12 may be configured to comply with regulatory acts such as HIPPA. For example, the admin network comms 152-12 may include features such as encryption, access control, audit trails, secure transmission, credential verification, or other features that may protect the data's integrity and privacy.

FIG. 104 illustrates an example operation of the device base module 124-12. The process may begin with the device base module 124-12 polling the Active Range RF signals between the one or more TX antennas 110-12 and the one or more RX antennas 111-12 at step 200-12. The device base module 124-12 may be configured to read and process instructions stored in the memory 114-12 using the processor 118-12. The one or more TX antennas 110-12 may be configured to transmit RF in the RF Activated Range from 500 MHZ to 300 GHZ. For example, the one or more TX antennas 110-12 may use RF signals at a range of 500 MHZ to 300 GHZ. The device base module 124-12 may receive the RF frequency signals from the one or more RX antennas 111-12 at step 202-12. For example, one or more RX antennas 111-12 receives a response or responded RF of frequency range 300-330 GHz from the patient's blood vessels. The device base module 124-12 may be configured to convert the received RF signals into a digital format using the ADC 112-12 at step 204-12. For example, the received RF of 300-330 GHZ frequency range is converted into a 10-bit data signal. The device base module 124-12 may be configured to store converted digital format into the memory 114-12 at step 206-12.

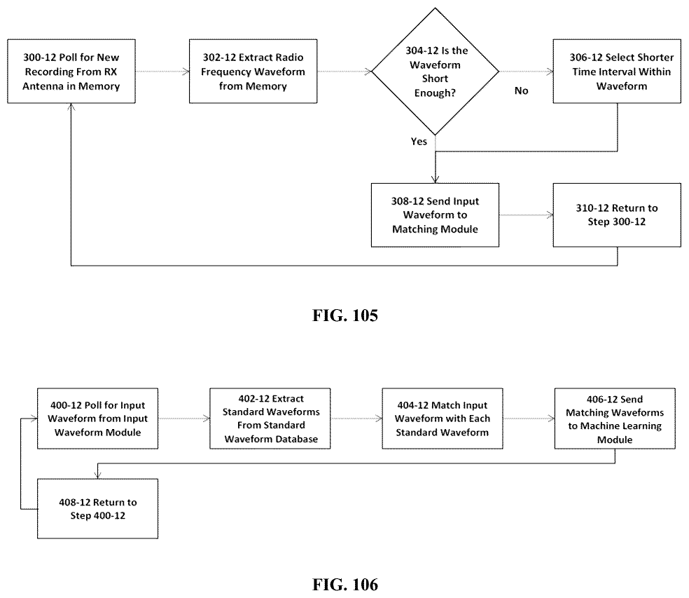

FIG. 105 illustrates an example operation of the input waveform module 126-12. The process may begin with the input waveform module 126-12 polling, at step 300-12, for new recorded data from the one or more RX antennas 111-12 stored in memory 114-12. The input waveform module 126-12 may extract, at step 302-12, the recorded radio frequency waveform from memory. If there is more than one waveform recorded, the input waveform module 126-12 may select each waveform separately and loop through the following steps. The input waveform module 126-12 may determine, at step 304-12, if the waveform is small enough to be an input waveform for the matching module 128-12. This will depend on the computational requirements and/or restrictions of the matching module 128-12. If the waveform is short enough, the input waveform module 126-12 may skip to step 308-12. If the waveform is too long, the input waveform module 126-12 may select, at step 306-12, a shorter time interval within the entire recorded waveform. For example, if the waveform is 5 minutes long, then only a 30-second interval may be selected. The interval may be selected at random or by a selection process. The input waveform module 126-12 may send, at step 308-12, the input waveform to the matching module 128-12. The input waveform module 126-12 may return, at step 310-12, to step 300-12.

FIG. 106 illustrates an example operation of the matching module 128-12. The process may begin with the matching module 128-12 polling, at step 400-12, for an input waveform from the input waveform module 126-12. The matching module 128-12 may extract, at step 402-12, each standard waveform from the standard waveform database 116-12. The matching module 128-12 may match, at step 404-12, the input waveform with each standard waveform. Matching may be determining which standard waveforms the input waveform is similar to. Matching may involve convolution and/or cross-correlation of the waveforms or any other matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair.

Convolution, on the other hand, is a mathematical operation that combines two functions to produce a third function. It is the integral of the product of two functions after one of them is flipped and then shifted. By applying convolution on two wave functions, the output will be a function whose values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130-12. The matching module 128-12 may send, at step 406-12, the matching waveforms to the machine learning module 130-12. Matching waveforms may refer to the standard waveforms that were similar to the input waveform, the waveforms that were generated via convolution and/or cross-correlation, or both. The matching module 128-12 may return, at step 408-12, to step 400-12.

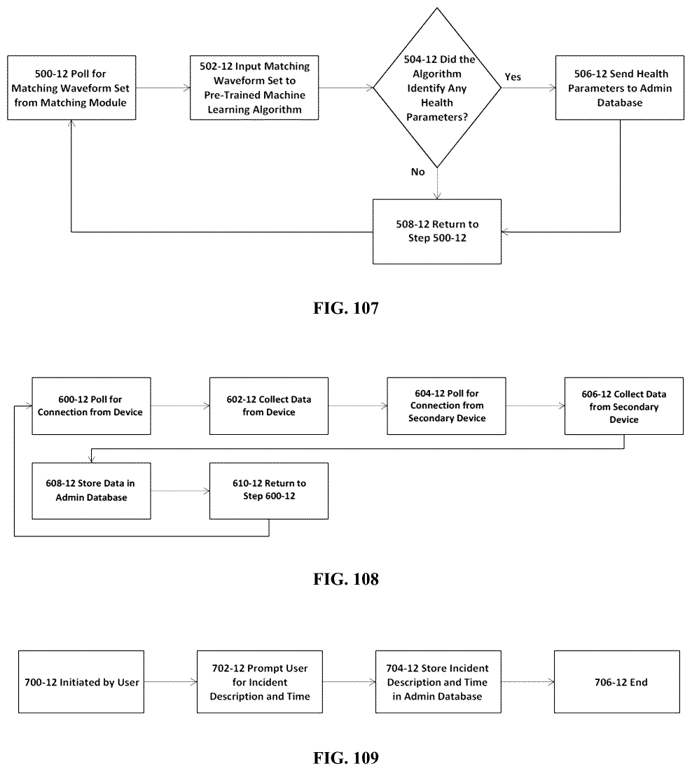

FIG. 107 illustrates an example operation of the machine learning module 130-12. The process may begin with the machine learning module 130-12 polling, at step 500-12, for a set of matching waveforms from the matching module 128-12. Matching waveforms may be a set of standard waveforms similar to the input waveform or statistical combinations of the input waveform and standard waveforms, such as convolutions or cross-correlations. The machine learning module 130-12 may input, at step 502-12, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During the training process, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130-12 may determine, at step 504-12, if the algorithm identified any health parameters. Identification may require a certain interval of confidence. For example, if the machine learning algorithm determines that it is more than 70% likely that a health parameter is correct, then that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130-12 may skip to step 508-12. If any health parameters were identified, the machine learning module 130-12 may send, at step 506-12, the health parameters to the admin database 138-12. The machine learning module 130-12 may return, at step 508-12, to step 500-12.

FIG. 108 illustrates an example operation of the connection module 140-12. The connection module 140-12 may poll, at step 600-12, for a connection from the device 108-12. If no connection is detected, the connection module 140-12 may search for a device 108-12 that is attempting to connect or may skip to step 604-12. The connection module 140-12 may collect, at step 602-12, data from the device 108-12. This may be real-time data or periodically updated data. This data may come directly from the machine learning module 130-12. The connection module 140-12 may poll, at step 604-12, for a connection from the industrial sensors 132-12. If no connection is detected, the connection module 140-12 may search for an industrial sensor 132-12 that is attempting to connect or may skip to step 608-12. The connection module 140-12 may begin at this step if an industrial sensor 132-12 is unavailable or unnecessary. The connection module 140-12 may collect, at step 606-12, data from the industrial sensors 132-12. This may be real-time data or periodically updated data. The connection module 140-12 may store, at step 604-12, the data from the device 108-12 and/or industrial sensors 132-12 in the admin database 138-12. The connection module 140-12 may add contextual data such as the ID of the industrial sensor 132-12, the ID of the device 108-12, the analyte measured by the device 108-12, the time data was retrieved, etc., to the received data before storage in the admin database 138-12. The connection module 140-12 may return, at step 610-12, to step 600-12.

FIG. 109 illustrates an example operation of the incident log module 142-12. The incident log module 142-12 may initiate, at step 700-12, the process upon receiving input from a user. For example, the user may select “log incident” from a menu on a webpage or within an application. The user may refer to a worker, manager, employee, administrator, etc. The incident log module 142-12 may prompt, at step 702-12, the user for incident description and time. The incident description may include the person or persons involved in the incident. The incident log module 142-12 may store, at step 704-12, the incident description and time in the admin database 138-12. The incident log module 142-12 may end at step 706-12.

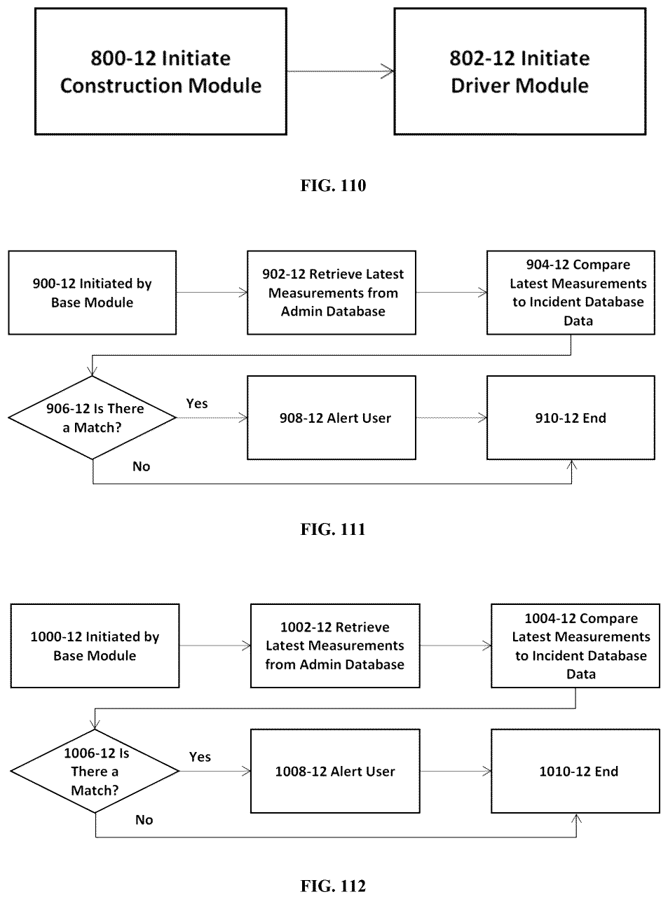

FIG. 110 illustrates an example operation of the base module 144-12. The base module 144-12 may initiate, at step 800-12, the construction module 146-12. The construction module 146-12 may predict construction incidents such as falls, injuries, and equipment failures due to user error based on the data from the device 108-12 and industrial sensors 132-12. The base module 144-12 may skip this step if no users work or manage a construction site or project. The base module 144-12 may initiate, at step 802-12, the driver module 148-12. The driver module 148-12 may predict driving incidents such as crashes based on the data from the device 108-12 and industrial sensors 132-12. The base module 144-12 may skip this step if no users drive or manage drivers.

FIG. 111 illustrates an example operation of the construction module 146-12. The construction module 146-12 may be initiated, at step 900-12, by the base module 144-12. The construction module 146-12 may retrieve, at step 902-12, the latest measurements of worker health conditions and construction activities from the admin database 138-12, which stores the data collected from the device 108-12 and industrial sensors 132-12. Examples of the latest measurements retrieved include worker health conditions such as blood sugar levels, heart rate, body temperature, and fatigue levels. For instance, the device 108-12 may collect data related to a worker's blood sugar level of 60 mg/dL, which may indicate hypoglycemia, a heart rate of 110 beats per minute (bpm), a body temperature of 101.5° F. (38.6° C.), and fatigue levels using sensors that monitor eye movement or other physiological indicators, such as reaction time. The construction module 146-12 may also retrieve data on construction activities, such as ladder climbing, machinery operation, environmental conditions, and worker movement. The industrial sensor 132-12, such as an accelerometer, may detect a worker's ladder climbing activity, including the rate of ascent at 0.5 m/s and the height reached at 3 meters. The industrial sensors 132-12 may also monitor a worker's interaction with construction machinery, such as the operation of a crane, including data on the load being lifted, the speed of movement, and the duration of the operation. Furthermore, the industrial sensors 132-12 may measure environmental factors at the construction site, such as ambient temperature at 95° F. (35° C.), humidity levels at 60% relative humidity, and noise levels at 85 decibels. The industrial sensors 132-12 may also track a worker's movement within the construction site, monitoring their location, speed, and the distance traveled during their shift. The construction module 146-12 may compare, at step 904-12, the latest measurements to historical data stored in the incident database 150-12, which contains records of previous construction incidents, accidents, and injuries. The construction module 146-12 may determine, at step 906-12, if there is a match or correlation between the latest measurements and the data from the incident database 150-12 by assessing the similarity of the current data from the device 108-12 and industrial sensor 132-12, such as an accelerometer, to historical data associated with previous incidents involving falls. The construction module 146-12 may identify a match if the data from the device 108-12 indicates that a worker has a low blood sugar level, for example, 60 mg/dL, and the data from the accelerometer demonstrates that the worker is climbing a ladder at a rate of 0.5 m/s. The module may then compare these latest measurements to the records in the incident database 150-12 that contain blood sugar levels and accelerometer readings from previous falls. The construction module 146-12 may employ one or more matching criteria, such as exact matches, matches falling within a pre-defined range, or matches exceeding the least extreme value of the historical data. The matching criteria may be based on statistical analysis, expert knowledge, or industry standards to ensure accurate and reliable predictions of potential risks. For example, the construction module 146-12 may identify a match if the worker's 60 mg/dL blood sugar level falls within a range of blood sugar levels associated with previous incidents, such as 55-65 mg/dL. Alternatively, the module may determine a match if the worker's blood sugar level is lower than the highest recorded value for previous incidents, for instance, 62 mg/dL, indicating an increased risk of falling. The construction module 146-12 may evaluate the accelerometer data to assess the similarity between the worker's current ladder-climbing activity and those recorded in previous incidents. This may involve analyzing the rate of ascent, such as 0.5 m/s, the height reached, or the ladder's stability, among other factors. If no match is found, the construction module 146-12 may skip to step 910-12. If there is a match, the construction module 146-12 may alert, at step 908-12, the worker, supervisor, and/or central control station, enabling appropriate intervention to ensure worker safety and prevent potential accidents or injuries. The alert may include the type of incident likely to occur, the person or persons at risk, where on the construction site the incident may occur, etc. The construction module 146-12 may end the process at step 910-12 and await further instructions from the base module 144-12 or initiate a new cycle to continuously monitor and predict construction incidents.

FIG. 112 illustrates an example operation of the driver module 148-12. The driver module 148-12 may be initiated, at step 1000-12, by the base module 144-12. The driver module 148-12 may retrieve, at step 1002-12, the latest measurements of driver health conditions and driving activities from the admin database 138-12, which stores the data collected from the device 108-12 and industrial sensors 132-12. Examples of the latest measurements retrieved include driver health conditions such as blood sugar levels, heart rate, body temperature, and fatigue levels. For instance, the device 108-12 may collect data related to a driver's blood sugar level of 60 mg/dL, which may indicate hypoglycemia, a heart rate of 110 beats per minute (bpm), a body temperature of 101.5° F. (38.6° C.), and fatigue levels using sensors that monitor eye movement or other physiological indicators, such as reaction time. The driver module 148-12 may also retrieve data on driving activities, such as vehicle speed, braking patterns, environmental conditions, and driver movement. The industrial sensor 132-12, such as an accelerometer, may detect a driver's vehicle speed, including the rate of acceleration at 0-60 mph in 5 seconds. The industrial sensors 132-12 may also monitor a driver's interaction with vehicle controls, such as braking patterns, including data on the force applied, the speed of deceleration, and the duration of braking. Furthermore, the industrial sensors 132-12 may measure environmental factors on the road, such as ambient temperature at 95° F. (35° C.), humidity levels at 60% relative humidity, and traffic conditions. The industrial sensors 132-12 may also track a driver's movement within the vehicle, monitoring their posture, attention level, and other behaviors during their shift. The driver module 148-12 may compare, at step 1004-12, the latest measurements to historical data stored in the incident database 150-12, which contains records of previous driving incidents, accidents, and injuries. The driver module 148-12 may determine, at step 1006-12, if there is a match or correlation between the latest measurements and the data from the incident database 150-12 by assessing the similarity of the current data from the device 108-12 and industrial sensor 132-12 to historical data associated with previous incidents involving driving accidents. The driver module 148-12 may identify a match if the data from the device 108-12 indicates that a driver has a low blood sugar level, for example, 60 mg/dL, and the data from the accelerometer demonstrates that the driver is accelerating rapidly at 0-60 mph in 5 seconds. The module may then compare these latest measurements to the records in the incident database 150-12 that contain blood sugar levels and accelerometer readings from previous accidents. The driver module 148-12 may employ one or more matching criteria, such as exact matches, matches falling within a pre-defined range, or matches exceeding the least extreme value of the historical data. The matching criteria may be based on statistical analysis, expert knowledge, or industry standards to ensure accurate and reliable predictions of potential risks. For example, the driver module 148-12 may identify a match if the driver's blood sugar level of 60 mg/dL falls within a range of blood sugar levels associated with previous incidents, such as 55-65 mg/dL. Alternatively, the module may determine a match if the driver's blood sugar level is lower than the highest recorded value for previous incidents, for instance, 62 mg/dL, indicating an increased risk of an accident. The driver module 148-12 may evaluate the accelerometer data to assess the similarity between the driver's current driving activity and the driving activities recorded in previous incidents. This may involve analyzing the rate of acceleration, such as 0-60 mph in 5 seconds, braking patterns, or the vehicle's stability, among other factors. If no match is found, the driver module 148-12 may skip to step 1010-12. If there is a match, the driver module 148-12 may alert, at step 1008-12, the driver, supervisor, and/or central control station, enabling appropriate intervention to ensure driver safety and prevent potential accidents or injuries. The alert may include the type of incident likely to occur, the person or persons at risk, where the incident may occur on the road, etc. The driver module 148-12 may end the process at step 1010-12, and await further instructions from the base module 144-12 or initiate a new cycle to continuously monitor and predict driving incidents.

In one example implementation of Embodiment 12, a system for measuring one or more analytes using a real-time, non-invasive radio frequency (“RF”) analyte detection device can include: the real-time, non-invasive RF analyte detection device; at least one sensor; and a database that contains historical incident data of at least one incident; wherein the database contains measurements from the real-time, non-invasive RF analyte detection device and the at least one sensor at the time of the at least one incident; wherein recent data from the real-time, non-invasive RF analyte detection device and the at least one sensor is compared to the historical incident data; and wherein at least one user is warned of an upcoming incident based on the comparison.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A health monitoring system, comprising;
an MRI device;
a non-invasive radio-frequency (RF) analyte detection device that includes at least one transmit antenna and at least one receive antenna, the at least one transmit antenna is positioned and arranged to transmit an RF signal into a user, and the at least one receive antenna is positioned and arranged to detect an RF response signal resulting from transmission of the RF signal by the at least one transmit antenna into the user;
a memory connected to and receiving data from the MRI device and the non-invasive RF analyte detection device;
a controller connected to and controlling the MRI device and the non-invasive RF analyte detection device;
a fusion module connected to the memory and that is configured to extract data therein from the MRI device and the non-invasive RF analyte detection device and that is configured to fuse the data;
wherein, the MRI device and the non-invasive RF device are controlled using the controller to put data collected by the MRI device and the non-invasive RF device in the memory and then execute the fusion module to fuse the collected data of both the MRI device and the non-invasive RF device.

2. The health monitoring system of claim 1, wherein the MRI device and the non-invasive RF analyte detection device are separate from one another.

3. A health monitoring method comprising:
a) collecting a first set of data from a target using a real-time, non-invasive radio-frequency (RF) analyte detection device that includes at least one transmit antenna and at least one receive antenna, the at least one transmit antenna is positioned and arranged to transmit an RF signal into the target, and the at least one receive antenna is positioned and arranged to detect an RF response signal resulting from transmission of the RF signal by the at least one transmit antenna into the target;
b) collecting a second set of data from the target using an invasive analyte detection device;
c) comparing the first set of data from the real-time, non-invasive RF analyte detection device to the second set of data from the invasive analyte detection device;
d) calibrating the real-time, non-invasive RF analyte detection device based on the comparison of the first set of data and the second set of data; and
e) repeating steps a)-d) until the real-time, non-invasive RF analyte detection device meets a compliance standard.

4. The method of claim 3, performing steps d) and e) on a periodic schedule.
